(19) United States
(12) Patent Application Publication
(10) Pub. No.: US 2004/0072860 A1
(43) Pub. Date: Apr. 15, 2004

(54) Piperazin-2-one amides as inhibitors of factor xa

(76) Inventors: Bing-Yan Zhu, Palo Alto, CA (US); Ting Su, Belmont, CA (US); Wenhao Li, South San Francisco, CA (US); Erik A. Goldman, Berkeley, CA (US); Penglie Zhang, Foster City, CA (US); Zhaozhong Jon Jia, San Mateo, CA (US); Robert M. Sacrborough, Half Moon Bay, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/381,927

(22) PCT Filed: Oct. 1, 2001
(86) PCT No.: PCT/US01/30313

(51) Int. Cl. A61K 31/44

(52) U.S. Cl. 514/295

(57) ABSTRACT

Novel piperazin-2-one containing compounds of general formulae (I) or (II), including their pharmaceutically acceptable isomers, salts, hydrates, solvates and prodrug derivative having activity against mammalian factor Xa arc described. Compositions containing such compounds are also described. The compounds and the compositions are useful in vitro or in vivo for preventing or treating conditions in mammals characterized by undesired thrombosis.

### FIELD OF THE INVENTION

The invention relates to novel piperazin-2-one-containing compounds including their pharmaceutically acceptable isomers, salts, hydrates, solvates and prodrug derivatives, and pharmaceutically acceptable compositions thereof which are potent and highly selective inhibitors of isolated factor Xa or when assembled in the prothrombinase complex. These compounds show selectivity for factor Xa versus other proteases of the coagulation (e.g. thrombin, fVIIa, flXa) or the fibrinolytic cascades (e.g. plasminogen activators, plasmin). In another aspect, the present invention relates to novel piperazin-2-one-containing compounds including their pharmaceutically acceptable isomers, salts, hydrates, solvates and prodrug derivatives factor Xa-inhibiting compounds, and pharmaceutically acceptable compositions thereof which are useful as potent and specific inhibitors of blood coagulation in mammals. In yet another aspect, the invention relates to methods for using these inhibitors as therapeutic agents for disease states in mammals characterized by undesired thrombosis or coagulation disorders.

### BACKGROUND OF THE INVENTION

Hemostasis, the control of bleeding, occurs by surgical means, or by the physiological properties of vasoconstriction and coagulation. The invention is particularly concerned with blood coagulation and ways in which it assists in maintaining the integrity of mammalian circulation after injury, inflammation, disease, congenital defect, dysfunction or other disruption. Although platelets and blood coagulation are both involved in thrombus formation, certain components of the coagulation cascade are primarily responsible for the amplification or acceleration of the processes involved in platelet aggregation and fibrin deposition.

Thrombin is a key enzyme in the coagulation cascade as well as in hemostasis. Thrombin plays a central role in thrombosis through its ability to catalyze the conversion of fibrinogen into fibrin and through its potent platelet activation activity. Direct or indirect inhibition of thrombin activity has been the focus of a variety of recent anticoagulant strategies as reviewed by Claeson, G., “Synthetic Peptides and Peptidomimetics as Substrates and Inhibitors of Thrombin and Other Proteases in the Blood Coagulation System”, Blood Coag. Fibrinol. 5, 411-436 (1994). Several classes of anticoagulants currently used in the clinic directly or indirectly affect thrombin (i.e. heparins, low-molecular weight heparins, heparin-like compounds and coumarins).

A prothrombinase complex, including Factor Xa (a serine protease, the activated form of its Factor X precursor and a member of the calcium ion binding, gamma carboxyglutamyl (Gla)-containing, vitamin K dependent, blood coagulation glycoprotein family), converts the zymogen prothrombin into the active procoagulant thrombin. Unlike thrombin, which acts on a variety of protein substrates as well as at a specific receptor, factor Xa appears to have a single physiologic substrate, namely prothrombin. Since one molecule of factor Xa may be able to generate up to 138 molecules of thrombin (Elodi et al., Thromb. Res. 15, 617-619 (1979)), direct inhibition of factor Xa as a way of indirectly inhibiting the formation of thrombin may be an efficient anticoagulant strategy. Therefore, it has been suggested that compounds which selectively inhibit factor Xa may be useful as in vitro diagnostic agents, or for therapeutic administration in certain thrombotic disorders, see e.g., WO 94/13693.

Polypeptides derived from hematophagous organisms have been reported which are highly potent and specific inhibitors of factor Xa. U.S. Pat. No. 4,588,587 describes anticoagulant activity in the saliva of the Mexican leech, Haementeria officinalis. A principal component of this saliva was shown to be the polypeptide factor Xa inhibitor, antistasin (ATS), by Nutt, E. et al, “The Amino Acid Sequence of Antistasin, a Potent Inhibitor of Factor Xa Reveals a Repeated Internal Structure”, J. Biol. Chem., 263, 10162-10167 (1988). Another potent and highly specific inhibitor of Factor Xa, called tick anticoagulant peptide (TAP), has been isolated from the whole body extract of the soft tick Ornithidoros moubata, as reported by Waxman, L., et al., “Tick Anticoagulant Peptide (TAP) is a Novel Inhibitor of Blood Coagulation Factor Xa” Science, 248, 593-596 (1990).

Factor Xa inhibitory compounds which are not large polypeptide-type inhibitors have also been reported including: Tidwell, R. R. et al., “Strategies for Anticoagulation With Synthetic Protease Inhibitors. Xa Inhibitors Versus Thrombin Inhibitors”, Thromb. Res., 19, 339-349 (1980); Turner, A. D. et al., “p-Amidino Esters as Irreversible Inhibitors of Factor IXa and Xa and Thrombin”, Biochemistry, 25, 4929-4935 (1986); Hitomi, Y. et al., “Inhibitory Effect of New Synthetic Protease Inhibitor (FUT-175) on the Coagulation System”, Haemostasis, 15, 164-168 (1985); Sturzebecher, J. et al., “Synthetic Inhibitors of Bovine Factor Xa and Thrombin. Comparison of Their Anticoagulant Efficiency”, Thromb. Res., 54, 245-252 (1989); Kam, C. M. et al., “Mechanism Based Isocoumarin Inhibitors for Trypsin and Blood Coagulation Serine Proteases: New Anticoagulants”, Biochemistry, 27, 2547-2557 (1988); Hauptmann, J. et al., “Comparison of the Anticoagulant and Antithrombotic Effects of Synthetic Thrombin and Factor Xa Inhibitors”, Thromb. Haemost., 63, 220-223 (1990); and the like.

Others have reported Factor Xa inhibitors which are small molecule organic compounds, such as nitrogen containing heterocyclic compounds which have amidino substituent groups, wherein two functional groups of the compounds can bind to Factor Xa at two of its active sites. For example, WO 98/28269 describes pyrazole compounds having a terminal C(═NH)—NH₂ group; WO 97/21437 describes benzimidazole compounds substituted by a basic radical which are connected to a naphthyl group via a straight or branched chain alkylene,—C(═O) or —S(═O)₂ bridging group; WO 99/10316 describes compounds having a 4-phenyl-N-alkylamidino-piperidine and 4-phenoxy-N-alkylamidino-piperidine group connected to a 3-amidinophenyl group via a carboxamidealkyleneamino bridge; and EP 798295 describes compounds having a 4-phenoxy-N-alkylamidino-piperidine group connected to an amidinonaphthyl group via a substituted or unsubstituted sulfonamide or carboxamide bridging group.

There exists a need for effective therapeutic agents for the regulation of hemostasis, and for the prevention and treatment of thrombus formation and other pathological processes in the vasculature induced by thrombin such as restenosis and inflammation. In particular, there continues to be a need for compounds which selectively inhibit factor Xa or its precursors. Compounds are needed which selectively or preferentially bind to Factor Xa. Compounds with a higher affinity for binding to Factor Xa than to thrombin are desired, especially those compounds having good bioavailability or other pharmacologically desirable properties.

### SUMMARY OF THE INVENTION

The present invention relates to novel piperazin-2-one-containing compounds including their pharmaceutically acceptable isomers, salts, hydrates, solvate and prodrug derivatives, which have particular biological properties and are useful as potent and specific inhibitors of blood coagulation in mammals. According to the invention, the compounds can act as potent and highly selective inhibitors of isolated Factor Xa or when assembled in the prothrombinase complex. The invention also provides compositions containing such compounds. The compounds of the invention may be used as diagnostic reagents or as therapeutic reagents for disease states in mammals which have coagulation disorders. Thus, the invention further provides methods for preventing or treating a condition in a mammal characterized by undesired thrombosis by administration of a therapeutically effective amount of a compound of the invention and a pharmaceutically acceptable carrier. Optionally, the methods of the invention comprise administering a pharmaceutical composition of the invention in combination with an additional therapeutic agent such as an antithrombotic and/or a thrombolytic agent and/or an anticoagulant. According to the invention, such conditions include, for example, any thrombotically mediated acute coronary or cerebrovascular syndrome, any thrombotic syndrome occurring in the venous system, any coagulopathy, and any thrombotic complications associated with extracorporeal circulation or instrumentation, and for the inhibition of coagulation in biological samples (e.g. stored blood products and samples).

The invention provides a compound of the general formulae I or II:

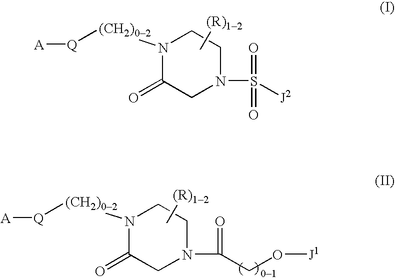

wherein:

A is a member selected from the group consisting of:

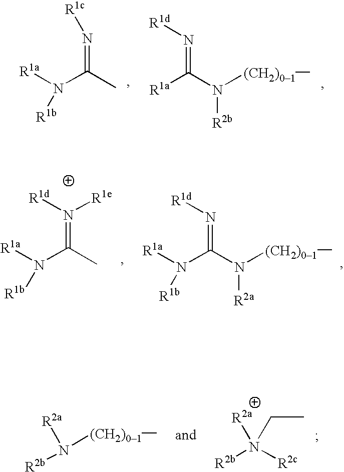

R^{1a}, R^{1b}, R^{1d}, and R^{1e} are each independently a H, C₁₋₆alkyl, C₂₋₆alkenyl, C₂₋₆alkynyl, C₃₋₈cycloalkyl, aryl, —C₁₋₆alkylaryl, heterocyclyl, —C₁₋₆alkylheterocyclyl, —(CH₂)₁₋₆OH, —(CH₂)₁₋₆OC₁₋₆alkyl, —(CH₂)₁₋₆NH₂, —(CH₂)₁₋₆NHC₁₋₆alkyl, —(CH₂)₁₋₆N(C₁₋₆alkyl)₂, —(CH₂)₁₋₆CHNH(COOH), —(CH₂)₁₋₆NHC(═O)C₁₋₆alkyl, —(CH₂)₁₋₆CHO, —(CH₂)₁₋₆C(═O)OH, —(CH₂)₁₋₆C(═O)OC₁₋₆alkyl, or —(CH₂)₁₋₆C(═O)NH₂; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide; or R^{1a} and R^{1b} or R^{1a} and R^{1c} or R^{1a} and R^{1d} or R^{1d} and R^{1e} taken together with the nitrogen atom to which they are each attached can form a substituted or unsubstituted 3 to 8 membered heterocyclic or heteroaromatic amine group which, optionally, contains at least one other heteroatom of N, O or S; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R^{1c} is H, C₁₋₆alkyl or C₃₋₈cycloalkyl;

R^{2a}, R^{2b} and R^{2c} are each independently a H, C₁₋₆alkyl, C₂₋₆alkenyl, C₂₋₆alkynyl, C₃₋₈cycloalkyl, aryl, —C₁₋₆alkylaryl, heterocyclyl, —C₁₋₆alkylheterocyclyl, —(CH₂)₁₋₆OH, —(CH₂)₁₋₆OC₁₋₆alkyl, —(CH₂)₁₋₆NH₂, —(CH₂)₁₋₆NHC₁₋₆alkyl, —(CH₂)₁₋₆N(C₁₋₆alkyl)₂, —(CH₂)₁₋₆CHNH(COOH), —(CH₂)₁₋₆NHC(═O)C₁₋₆alkyl, —(CH₂)₁₋₆CHO, —(CH₂)₁₋₆C(═O)OH, —(CH₂)₁₋₆C(═O)OC₁₋₆alkyl, or —(CH₂)₁₋₆C(═O)NH₂; wherein R^{2a}, R^{2b} and R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide; or R^{2a}, and R^{2b} or R^{1a}, as set forth above, and R^{2a} or R^{1a}, as set forth above, and R^{2b} taken together with the nitrogen atom to which they are each attached can form a substituted or unsubstituted 3 to 8 membered heterocyclic or heteroaromatic amine group which, optionally, contains at least one other heteroatom of N, O or S; wherein R^{2a}, R^{2b} or R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R is, in each occurrence, independently, H, —C₁₋₆alkyl, —C₃₋₈cycloalkyl, —C₀₋₆alkyl-OC₁₋₆alkyl, —C₀₋₆alkyl-O(CH₂)₁₋₄—COOH, —C₀₋₆alkyl-O(CH₂)₁₋₄—C(═O)OC₁-C₆alkyl, —C₀₋₆alkylCOOH, —C₀₋₆alkylCO₂C₁₋₆alkyl, —C₀₋₆alkylOC₁₋₆alkyl, —C₁₋₆alkylOH, —C₀₋₆alkylCONH₂, —C₀₋₆alkylCONHC₀₋₆alkyl, —C₀₋₆alkylCON(C₀₋₆alky)₂, —C₀₋₆alkylCON(CH₂)₂₋₆, —C₀₋₆alkylCON(CH₂CH₂)₂O, —C₀₋₆alkylCON(CH₂CH₂)₂SO₂ —C₀₋₆alkylCONHaryl, —C₀₋₆alkylNH₂, —C₀₋₆alkylNH(C₁₋₆alkyl) or —C₀₋₆alkylN(C₁₋₆alkyl)₂.

Q is a member selected from the group consisting of:

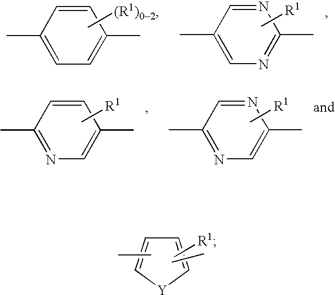

Y is S;

R¹ is H, —Cl, —Br, —I, —F, —OCF₃, alkyl, hydroxy, alkoxy, amino, thiol, thioalkyl, thioaryl, or piperizinyl;

J¹ is a member selected from the group consisting of:

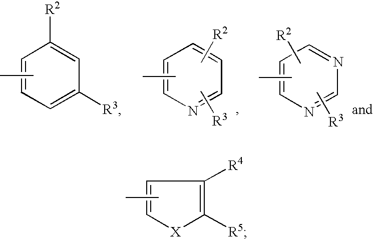

X is O or S;

R² is H, —Cl, —Br, —I, —F or —OC₁₋₆alkyl;

R³ is H, —Cl, —Br, —I, —F, —OC₁₋₆alkyl, —NHC₁₋₆acyl, —NO₂, —NHSO₂C₁₋₄alkyl, —CN, —NH₂, —CONH₂, —SO₂C₁₋₆alkyl, —SO₂NH₂, —CO₂C₁₋₆alkyl or —O(CH₂)₁₋₄COOH;

R⁴ and R⁵ are each independently H, —Cl, —Br, —I, —F or —OC₁₋₆alkyl;

j² is a member selected from the group consisting of:

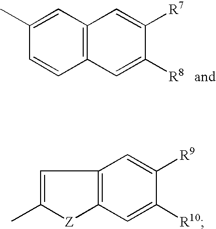

Z is —NR⁶—, —O— or —S—;

R⁶ H, C₁₋₆alkyl or C₃₋₈cycloalkyl;

R⁷ and R⁸ are independently H, —Cl, —Br, —I or —F, where at least one of R⁷ and R⁸ is not hydrogen; and

R⁹ and R¹⁰ are independently H, —Cl, —Br, —I or —F, where at least one of R⁹ and R¹⁰ is not hydrogen;

and all pharmaceutically acceptable isomers, salts, hydrates, solvates and prodrug derivatives thereof.

### DETAILED DESCRIPTION OF THE INVENTION

Definitions

In accordance with the present invention and as used herein, the following terms are defined with the following meanings, unless explicitly stated otherwise.

The term “alkenyl” refers to a trivalent straight chain or branched chain unsaturated aliphatic radical. The term “alkynyl” (or “alkinyl”) refers to a straight or branched chain aliphatic radical that includes at least two carbons joined by a triple bond. If no number of carbons is specified alkenyl and alkynyl each refer to radicals having from 2-12 carbon atoms.

The term “alkyl” refers to saturated aliphatic groups including straight-chain, branched-chain and cyclic groups having the number of carbon atoms specified, or if no number is specified, having up to 12 carbon atoms. The term “cycloalkyl” as used herein refers to a mono-, bi-, or tricyclic aliphatic ring having 3 to 14 carbon atoms and preferably 3 to 7 carbon atoms.

As used herein, the terms “carbocyclic ring structure” and “C₃₋₁₆ carbocyclic mono, bicyclic or tricyclic ring structure” or the like are each intended to mean stable ring structures having only carbon atoms as ring atoms wherein the ring structure is a substituted or unsubstituted member selected from the group consisting of: a stable monocyclic ring which is aromatic ring (“aryl”) having six ring atoms; a stable monocyclic non-aromatic ring having from 3 to 7 ring atoms in the ring; a stable bicyclic ring structure having a total of from 7 to 12 ring atoms in the two rings wherein the bicyclic ring structure is selected from the group consisting of ring structures in which both of the rings are aromatic, ring structures in which one of the rings is aromatic and ring structures in which both of the rings are non-aromatic; and a stable tricyclic ring structure having a total of from 10 to 16 atoms in the three rings wherein the tricyclic ring structure is selected from the group consisting of: ring structures in which three of the rings are aromatic, ring structures in which two of the rings are aromatic and ring structures in which three of the rings are non-aromatic. In each case, the non-aromatic rings when present in the monocyclic, bicyclic or tricyclic ring structure may independently be saturated, partially saturated or fully saturated. Examples of such carbocyclic ring structures include, but are not limited to, cyclopropyl, cyclobutyl, cyclopentyl, cyclohexyl, adamantyl, cyclooctyl, [3.3.0]bicyclooctane, [4.3.0]bicyclononane, [4.4.0]bicyclodecane (decalin), 2.2.2]bicyclooctane, fluorenyl, phenyl, naphthyl, indanyl, adamantyl, or tetrahydronaphthyl (tetralin). Moreover, the ring structures described herein may be attached to one or more indicated pendant groups via any carbon atom which results in a stable structure. The term “substituted” as used in conjunction with carbocyclic ring structures means that hydrogen atoms attached to the ring carbon atoms of ring structures described herein may be substituted by one or more of the substituents indicated for that structure if such substitution(s) would result in a stable compound.

The term “aryl” which is included with the term “carbocyclic ring structure” refers to an unsubstituted or substituted aromatic ring, substituted with one, two or three substituents selected from loweralkoxy, loweralkyl, loweralkylamino, hydroxy, halogen, cyano, hydroxyl, mercapto, nitro, thioalkoxy, carboxaldehyde, carboxyl, carboalkoxy and carboxamide, including but not limited to carbocyclic aryl, heterocyclic aryl, and biaryl groups and the like, all of which may be optionally substituted. Examples of suitable aryl groups include, but are not limited to, phenyl, pyridyl, thiophenyl, halophenyl, loweralkylphenyl, naphthyl, biphenyl, phenanthrenyl and naphthacenyl.

The term “arylalkyl” which is included with the term “carbocyclic aryl” refers to one, two, or three aryl groups having the number of carbon atoms designated, appended to an alkyl group having the number of carbon atoms designated. Suitable arylalkyl groups include, but are not limited to, benzyl, picolyl, naphthylmetbyl, phenethyl, benzyhydryl, trityl, and the like, all of which may be optionally substituted.

As used herein, the term “heterocyclic ring” or “heterocyclic ring system” is intended to mean a substituted or unsubstituted member selected from the group consisting of stable monocyclic ring having from 5-7 members in the ring itself and having from 1 to 4 hetero ring atoms selected from the group consisting of N, O and S; a stable bicyclic ring structure having a total of from 7 to 12 atoms in the two rings wherein at least one of the two rings has from 1 to 4 hetero atoms selected from N, O and S, including bicyclic ring structures wherein any of the described stable monocyclic heterocyclic rings is fused to a hexane or benzene ring; and a stable tricyclic heterocyclic ring structure having a total of from 10 to 16 atoms in the three rings wherein at least one of the three rings has from 1 to 4 hetero atoms selected from the group consisting of N, O and S. Any nitrogen and sulfur atoms present in a heterocyclic ring of such a heterocyclic ring structure may be oxidized. Unless indicated otherwise the terms “heterocyclic ring” or “heterocyclic ring system” include aromatic rings, as well as non-aromatic rings which can be saturated, partially saturated or fully saturated non-aromatic rings. Also, unless indicated otherwise the term “heterocyclic ring system” includes ring structures wherein all of the rings contain at least one hetero atom as well as structures having less than all of the rings in the ring structure containing at least one hetero atom, for example bicyclic ring structures wherein one ring is a benzene ring and one of the rings has one or more hetero atoms are included within the term “heterocyclic ring systems” as well as bicyclic ring structures wherein each of the two rings has at least one hetero atom. Moreover, the ring structures described herein may be attached to one or more indicated pendant groups via any hetero atom or carbon atom which results in a stable structure. Further, the term “substituted” means that one or more of the hydrogen atoms on the ring carbon atom(s) or nitrogen atom(s) of the each of the rings in the ring structures described herein may be replaced by one or more of the indicated substituents if such replacement(s) would result in a stable compound. Nitrogen atoms in a ring structure may be quaternized, but such compounds are specifically indicated or are included within the term “a pharmaceutically acceptable salt” for a particular compound. When the total number of O and S atoms in a single heterocyclic ring is greater than, it is preferred that such atoms not be adjacent to one another. Preferably, there are no more that 1 O or S ring atoms in the same ring of a given heterocyclic ring structure.

Examples of monocyclic and bicyclic heterocyclic ring systems, in alphabetical order, are acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyroazolidinyl, pyrazolinyl, pyrazolyl, pyridazinyl, pryidooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Preferred heterocyclic ring structures include, but are not limited to, pyridinyl, furanyl, thienyl, pyrrolyl, pyrazolyl, pyrrolidinyl, imidazolyl, indolyl, benzimidazolyl, 1H-indazolyl, oxazolinyl, or isatinoyl. Also included are fused ring and spiro compounds containing, for example, the above heterocyclic ring structures.

As used herein the term “aromatic heterocyclic ring system” has essentially the same definition as for the monocyclic and bicyclic ring systems except that at least one ring of the ring system is an aromatic heterocyclic ring or the bicyclic ring has an aromatic or non-aromatic heterocyclic ring fused to an aromatic carbocyclic ring structure.

The terms “halo” or “halogen” as used herein refer to Cl, Br, F or I substituents. The term “haloalkyl”, and the like, refer to an aliphatic carbon radicals having at least one hydrogen atom replaced by a Cl, Br, F or I atom, including mixtures of different halo atoms. Trihaloalkyl includes trifluoromethyl and the like as preferred radicals, for example.

The term “methylene” refers to —CH₂—.

The term “pharmaceutically acceptable salts” includes salts of compounds derived from the combination of a compound and an organic or inorganic acid. These compounds are useful in both free base and salt form. In practice, the use of the salt form amounts to use of the base form; both acid and base addition salts are within the scope of the present invention.

“Pharmaceutically acceptable acid addition salt” refers to salts retaining the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic nontoxic bases are isopropylamine, diethylamine, ethanolamine, trimethamine, dicyclohexylamine, choline, and caffeine.

“Biological property” for the purposes herein means an in vivo effector or antigenic function or activity that is directly or indirectly performed by a compound of the invention that are often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

In the compounds of the invention, carbon atoms bonded to four non-identical substituents are asymmetric. Accordingly, the compounds may exist as diastereoisomers, enantiomers or mixtures thereof. The syntheses described herein may employ racemates, enantiomers or diastereomers as starting materials or intermediates. Diastereomeric products resulting from such syntheses may be separated by chromatographic or crystallization methods, or by other methods known in the art. Likewise, enantiomeric product mixtures may be separated using the same techniques or by other methods known in the art. Each of the asymmetric carbon atoms, when present in the compounds of the invention, may be in one of two configurations (R or S) and both are within the scope of the present invention.

Compounds

The invention provides a compound of the general formulae I or II:

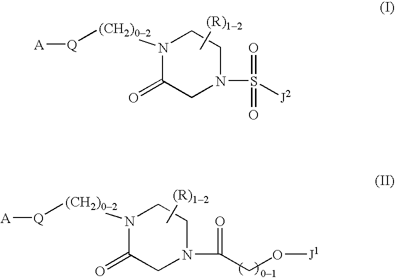

wherein:

A is a member selected from the group consisting of:

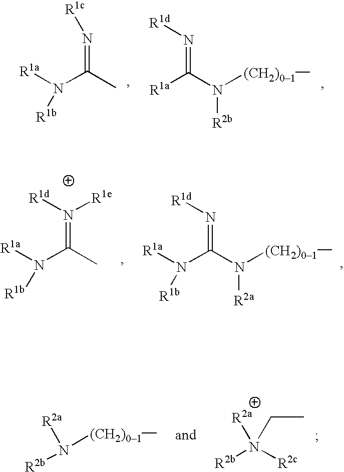

R^{1a}, R^{1b}, R^{1d}, and R^{1e} are each independently a H, C₁₋₆alkyl, C₂₋₆alkenyl, C₂₋₆alkynyl, C₃₋₈cycloalkyl, aryl, —C₁₋₆alkylaryl, heterocyclyl, —C₁₋₆alkylheterocyclyl, —(CH₂)₁₋₆OH, —(CH₂)₁₋₆OC₁₋₆alkyl, —(CH₂)₁₋₆NH₂, —(CH₂)₁₋₆NHC₁₋₆alkyl, —(CH₂)₁₋₆N(C₁₋₆alkyl)₂, —(CH₂)₁₋₆CHNH(COOH), —(CH₂)₁₋₆NHC(═O)C₁₋₆alkyl, —(CH₂)₁₋₆CHO, —(CH₂)₁₋₆C(═O)OH, —(CH₂)₁₋₆C(═O)OC₁₋₆alkyl, or —(CH₂)₁₋₆C(═O)NH₂; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide; or R^{1a} and R^{1b} or R^{1a} and R^{1c} or R^{1a} and R^{1d} or R^{1d} and R^{1e} taken together with the nitrogen atom to which they are each attached can form a substituted or unsubstituted 3 to 8 membered heterocyclic or heteroaromatic amine group which, optionally, contains at least one other heteroatom of N, O or S; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R^{1c} is H, C₁₋₆alkyl or C₃₋₈cycloalkyl;

R^{2a}, R^{2b} and R^{2c} are each independently a H, C₁₋₆alkyl, C₂₋₆alkenyl, C₂₋₆alkynyl, C₃₋₈cycloalkyl, aryl, —C₁₋₆alkylaryl, heterocyclyl, —C₁₋₆alkylheterocyclyl, —(CH₂)₁₋₆OH, —(CH₂)₁₋₆OC₁₋₆alkyl, —(CH₂)₁₋₆NH₂, —(CH₂)₁₋₆NHC₁₋₆alkyl, —(CH₂)₁₋₆N(C₁₋₆alkyl)₂, —(CH₂)₁₋₆CHNH(COOH), —(CH₂)₁₋₆NHC(═O)C₁₋₆alkyl, —(CH₂)₁₋₆CHO, —(CH₂)₁₋₆C(═O)OH, —(CH₂)₁₋₆C(═O)OC₁₋₆alkyl, or —(CH₂)₁₋₆C(═O)NH₂; wherein R^{2a}, R^{2b} and R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide; or R^{2a} and R^{2b} or R^{1a}, as set forth above, and R^{2a} or R^{1a}, as set forth above, and R^{2b} taken together with the nitrogen atom to which they are each attached can form a substituted or unsubstituted 3 to 8 membered heterocyclic or heteroaromatic amine group which, optionally, contains at least one other heteroatom of N, O or S; wherein R^{2a}, R^{2b} or R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;

R is, in each occurrence, independently, H, —C₁₋₆alkyl, —C₃₋₈cycloalkyl, —C₀₋₆alkyl-OC₁₋₆alkyl, —C₀₋₆alkyl-O(CH₂)₁₋₄—COOH, —C₀₋₆alkyl-O(CH₂)₁₋₄—C(═O)OC₁-C₆alkyl, —C₀₋₆alkylCOOH, —C₀₋₆alkylCO₂C₁₋₆alkyl, —C₀₋₆alkylOC₁₋₆alkyl, —C₁₋₆alkylOH, —C₀₋₆alkylCONH₂, —C₀₋₆alkyCONHC₀₋₆alkyl, —C₀₋₆alkylCON(C₀₋₆alky)₂, —C₀₋₆alkylCON(CH₂)₂₋₆, —C₀₋₆alkylNH₂, —C₀₋₆alkylNH(C₁₋₆alkyl) or —C₀₋₆alkylN(C₁₋₆alkyl)₂.

Q is a member selected from the group consisting of:

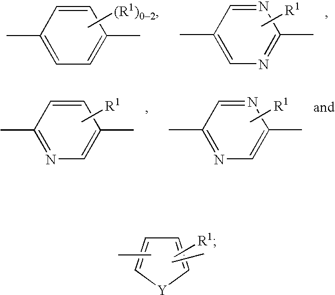

Y is S;

R¹ is H, —Cl, —Br, —I, —F, —OCF₃, alkyl, hydroxy, alkoxy, amino, thiol, thioalkyl, thioaryl, or piperizinyl;

J¹ is a member selected from the group consisting of:

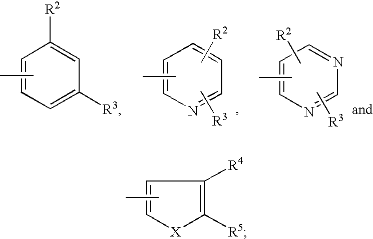

X is O or S;

R² is H, —Cl, —Br, —I, —F or —OC₁₋₆alkyl;

R³ is H, —Cl, —Br, —I, —F, —OC₁₋₆alkyl, —NHC₁₋₆acyl, —NO₂, —NHSO₂C₁₋₄alkyl, —CN, —NH₂, —CONH₂, —SO₂C₁₋₆alkyl, —SO₂NH₂, —CO₂C₁₋₆alkyl or —O(CH₂)₁₋₄COOH;

R⁴ and R⁵ are each independently H, —Cl, —Br, —I, —F or —OC₁₋₆alkyl;

J² is a member selected from the group consisting of:

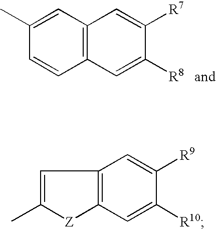

Z is —NR⁶—, —O— or —S—;

R⁶ is H, C₁₋₆alkyl or C₃₋₈cycloalkyl;

R⁷ and R⁸ are independently H, —Cl, —Br, —I or —F, where at least one of R⁷ and R8 is not hydrogen; and

R⁹ and R¹⁰ are independently H, —Cl, —Br, —I or —F, where at least one of R⁹ and R¹⁰ is not hydrogen;

and all pharmaceutically acceptable isomers, salts, hydrates, solvates and prodrug derivatives thereof.

The invention further provides a compound of the formulae (I) or (II):

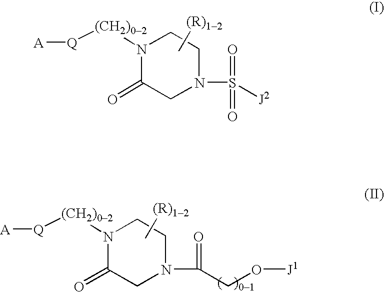

wherein:

A is a member selected from the group consisting of:

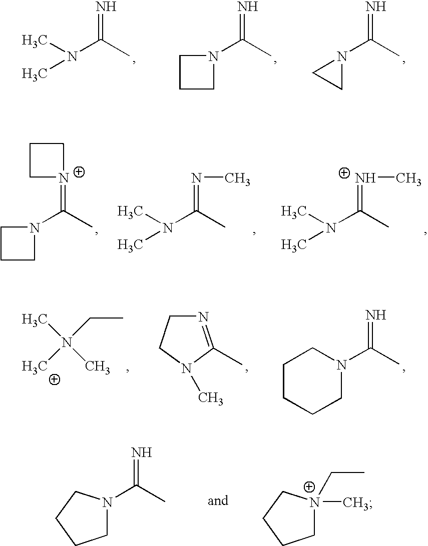

R is, in each occurrence, independently, H, —C₁₋₆alkyl, —C₃₋₈cycloalkyl, —C₁₋₆alkylOH, —C₀₋₆alkyl-OC₁₋₆alkyl, —C₀₋₆alkyl-O(CH₂)₁₋₄—COOH, —C₀₋₆alkyl-O(CH₂)₁₋₄—C(═O)OC₁—C₆alkyl, —C₀₋₆alkylCOOH, —C₀₋₆alkylCO₂C₁₋₆alkyl, —C₀₋₆alkylCONH₂,

—C₀₋₆alkylCONHC₀₋₆alkyl, —C₀₋₆alkylCON(C₀₋₆alky)₂, —C₀₋₆alkylCON(CH₂)₂₋₆, 13 C₀₋₆alkylNH₂, —C₀₋₆alkylNH(C₁₋₆alkyl) or —C₀₋₆alkylN(C₁₋₆alkyl)₂. Q is a member selected from the group consisting of:

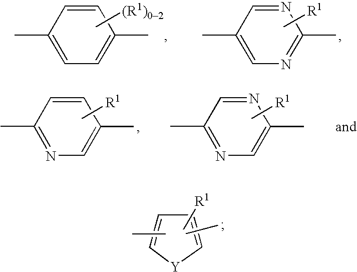

Y is S;

R¹ is H, —Cl, —Br, —I or —F, —OMe, NH2, NHMe, NHMe₂, —NHCOMe, —NHSO₂Me;

J¹ is a member selected from the group consisting of:

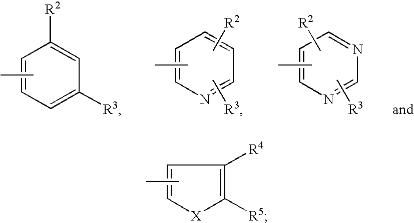

X is O or S;

R² is H, —Cl, —Br, —I, —F or —OC₁₋₆alkyl;

R³ is H, —Cl, —Br, —I, —F, —OC₁₋₆alkyl, —NHC₁₋₆acyl, —NO₂, —NHSO₂C₁₋₄alkyl, —CN or —O(CH₂)₁₋₄—COOH;

R⁴ and R⁵ are each independently H, —Cl, —Br, —I, —F or —OC₁₋₆alkyl;

J² is a member selected from the group consisting of:

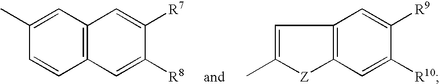

Z is —NR⁶—, —O— or —S—;

R⁶ is H, C₁₋₆alkyl or C₃₋₈cycloalkyl;

R⁷ and R⁸ are each independently H, —Cl, —Br, —I or —F, where at least one of R⁷ and R⁸ is not hydrogen; and

R⁹ and R¹⁰ are each independently H, —Cl, —Br, —I or —F, where at least one of R⁹ and R¹⁰ is not hydrogen;

and all pharmaceutically acceptable isomers, salts, hydrates, solvates and prodrug derivatives thereof.

The invention further provides a compound of formula (I) or (II):

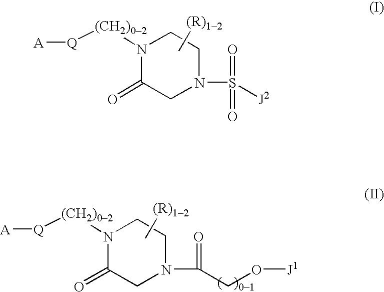

wherein:

A is a member selected from the group consisting of:

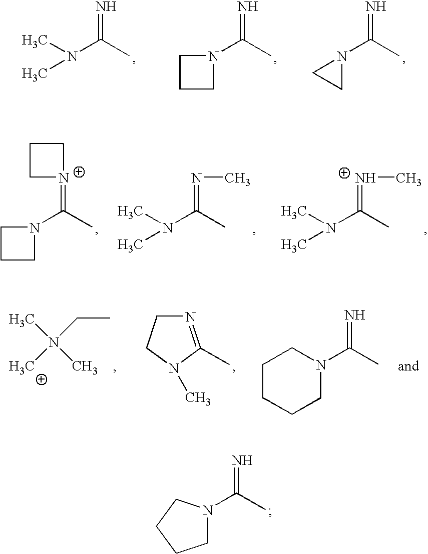

R is, in each occurrence, independently, H, —C₁₋₆alkyl, —C₃₋₈cycloalkyl, —C₁₋₆alkylOH, —C₀₋₆alkyl-OC₁₋₆alkyl, —C₀₋₆alkyl-O(CH₂)₁₋₄—COOH, —C₀₋₆alkyl-O(CH₂)₁₋₄—C(═O)OC₁-C₆alkyl, —C₀₋₆alkylCOOH, —C₀₋₆alkylCO₂C₁₋₆alkyl, —C₀₋₆alkylCONH₂, —C₀₋₆alkylCONHC₀₋₆alkyl, —C₀₋₆alkylCON(C₀₋₆alky)₂, —C₀₋₆alkylCON(CH₂)₂₋₆, —C₀₋₆alkylNH₂, —C₀₋₆alkylNH(C₁₋₆alkyl) or —C₀₋₆alkylN(C₁₋₆alkyl)₂.

Q is a member selected from the group consisting of:

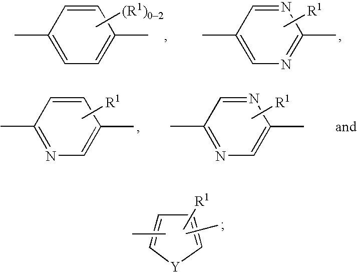

Y is S;

R¹ is H, —Cl, —Br, —I or —F, —OMe, NH₂, NHMe, NHMe₂;

J¹ is a member selected from the group consisting of:

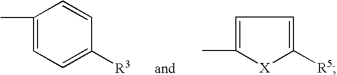

X is O or S;

R³ is H, —Cl, —Br, —I or —F;

R⁵ is H, —Cl, —Br, —I or —F;

J² is a member selected from the group consisting of:

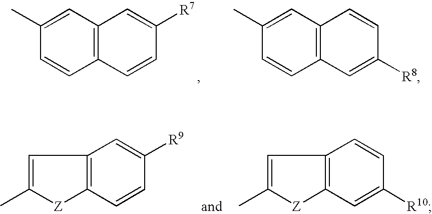

Z is —NR⁶—, —O— or —S—;

R⁶ is a H, C₁₋₆ alkyl or C₃₋₈ cycloalkyl;

R⁷ and R⁸ are each independently —Cl, —Br, —I or —F; and

R⁹ and R¹⁰ are each independently —Cl, —Br, —I or —F;

and all pharmaceutically acceptable isomers, salts, hydrates, solvates and prodrug derivatives thereof.

The invention provides compounds of formula (I) having the following structure:

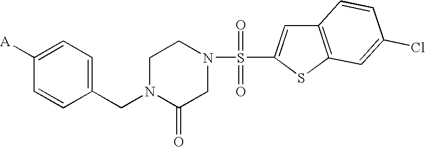

wherein:

A is a member selected from the group consisting of:

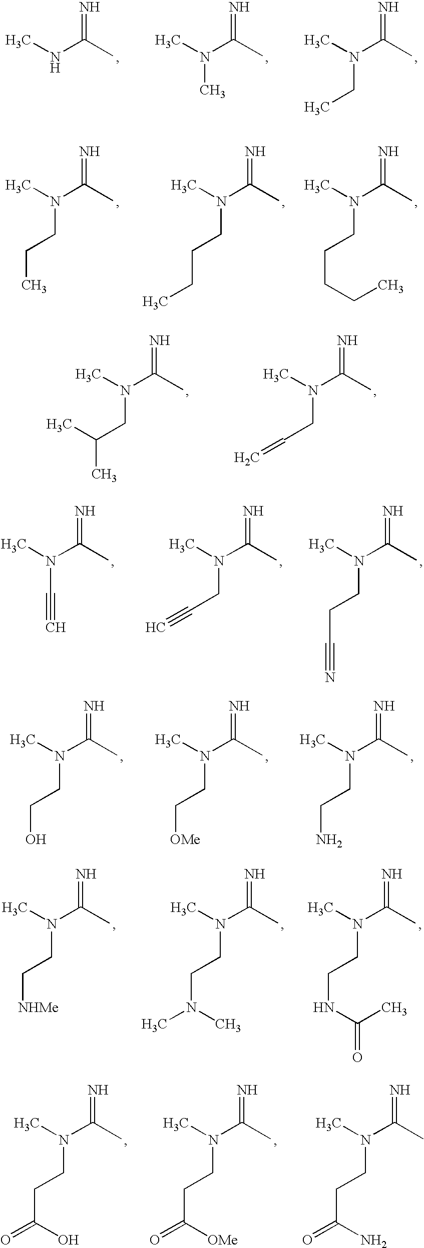

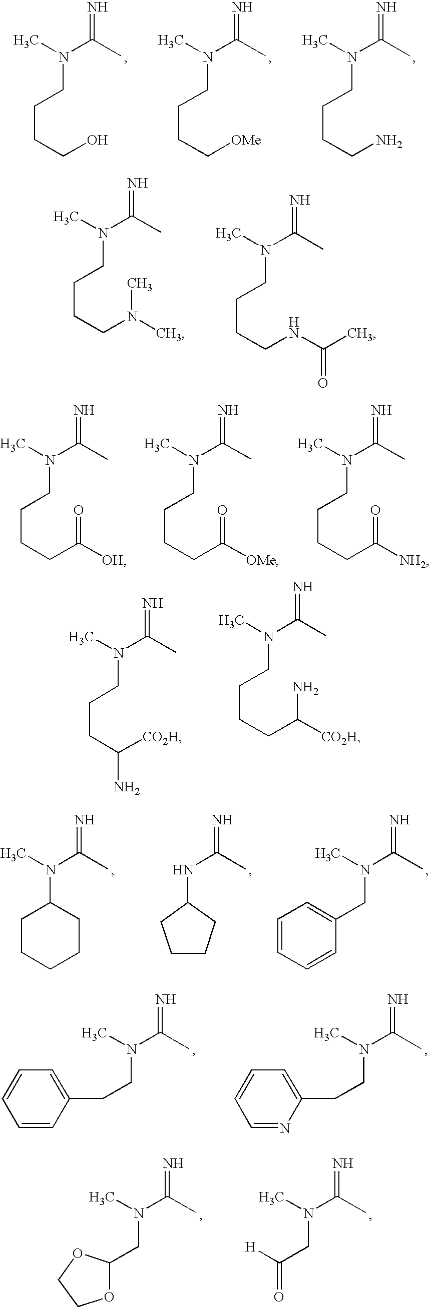

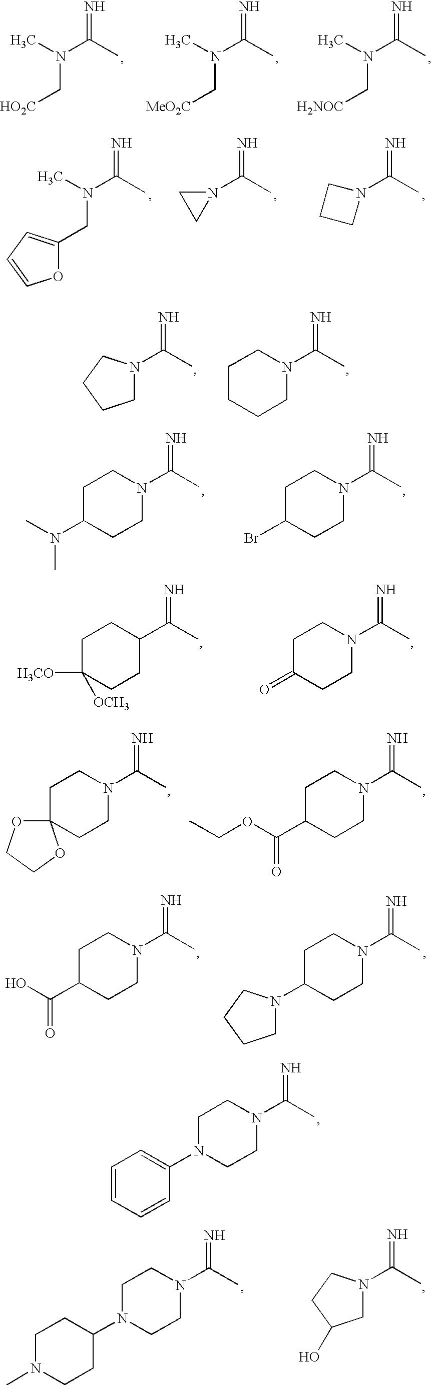

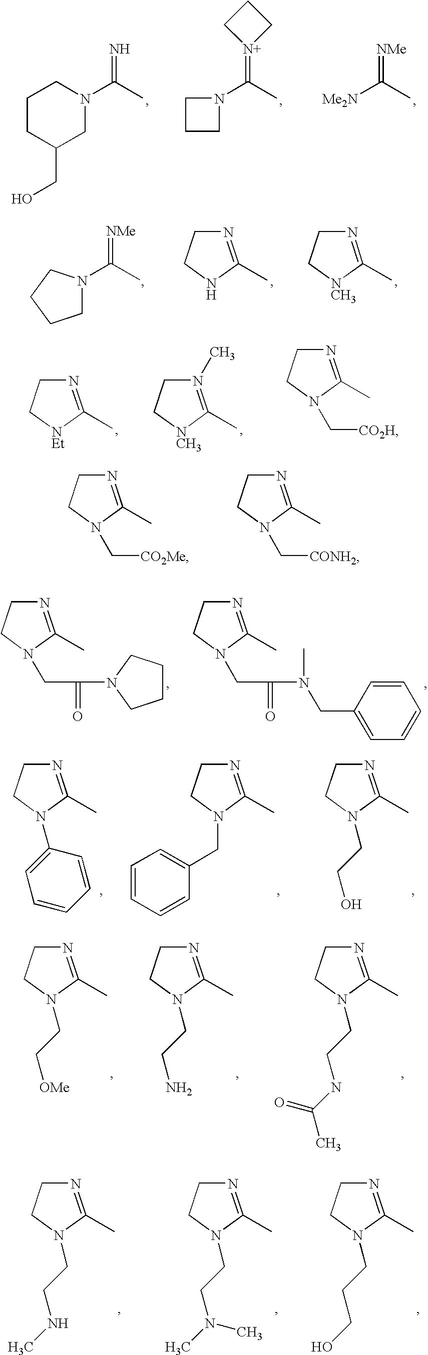

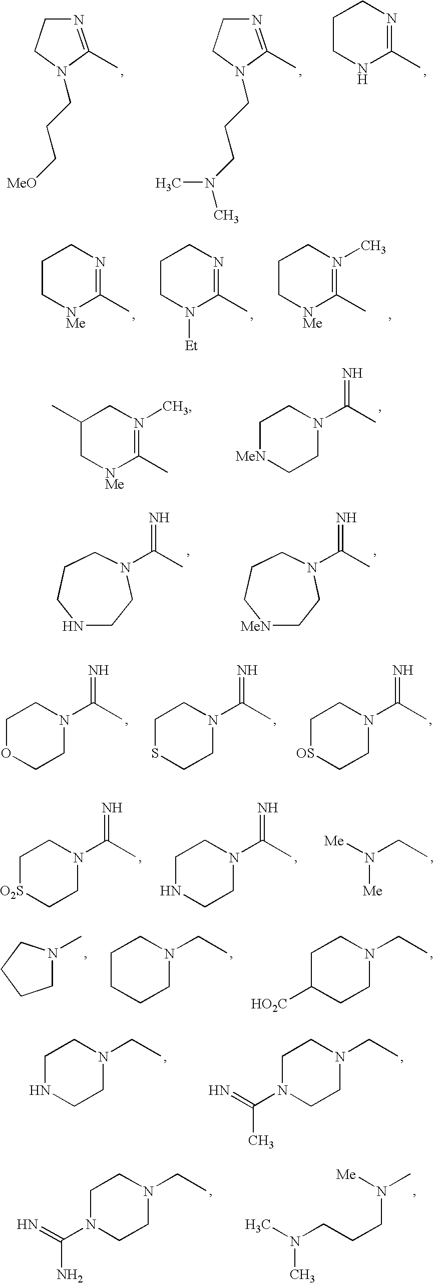

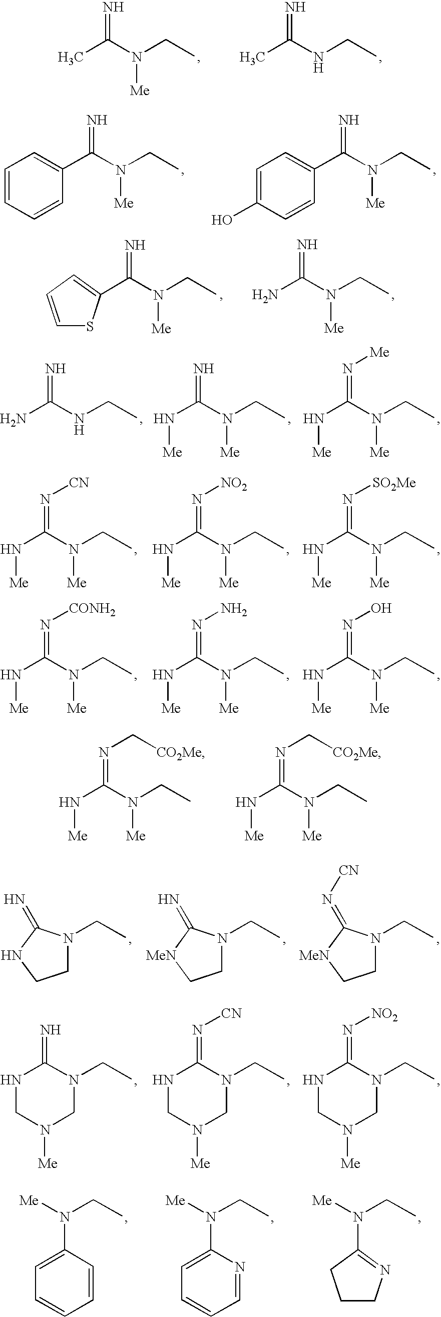

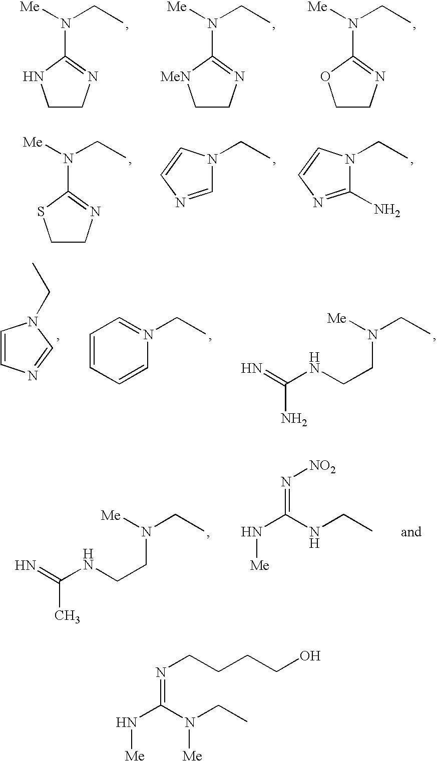

and all pharmaceutically acceptable isomers, salts, hydrates, solvates and prodrug derivatives thereof.

The invention further provides compounds of formula (I) having the following structure:

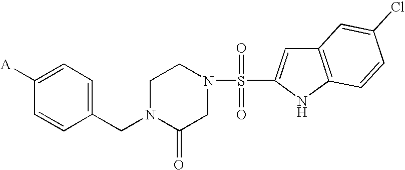

wherein:

A is a member selected from the group consisting of:

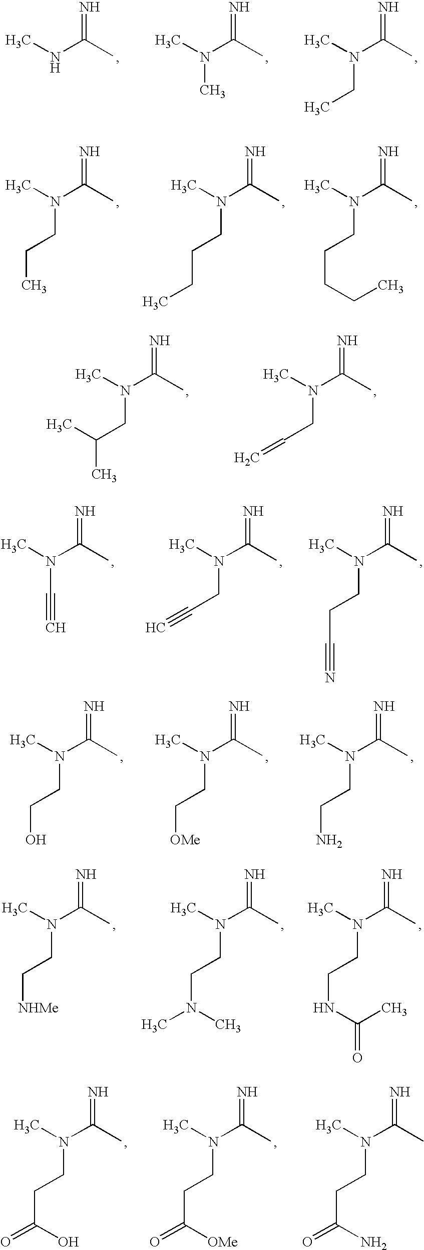

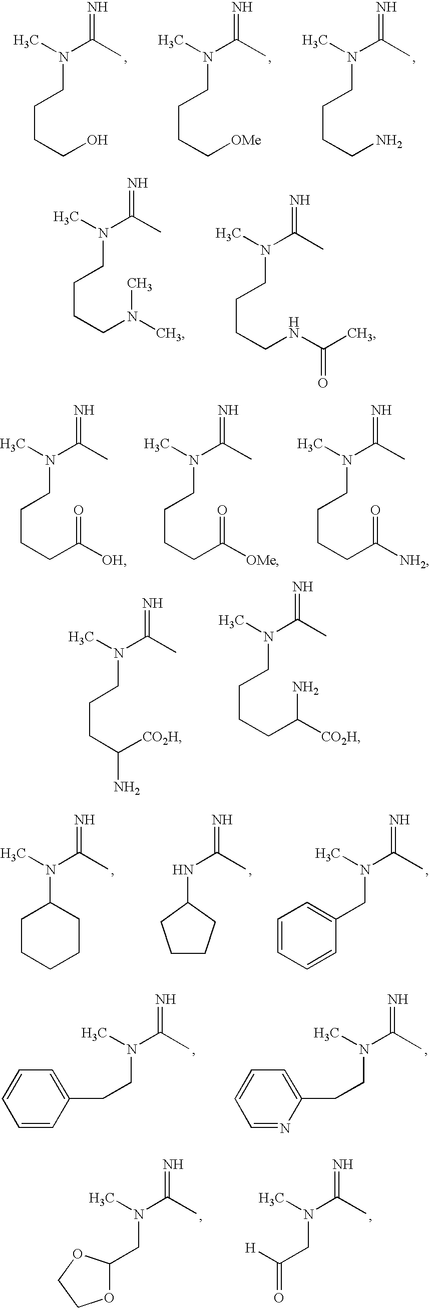

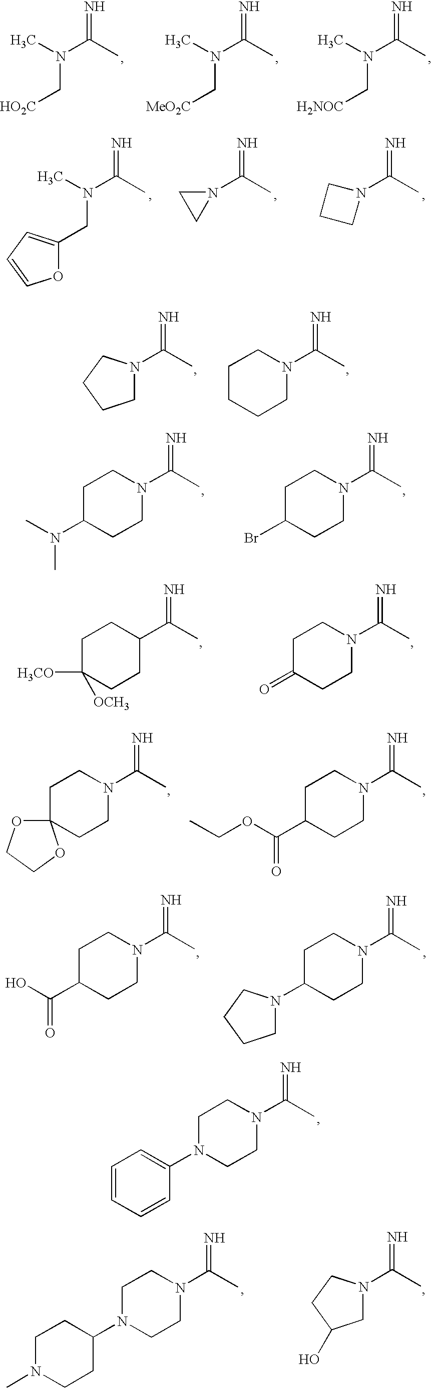

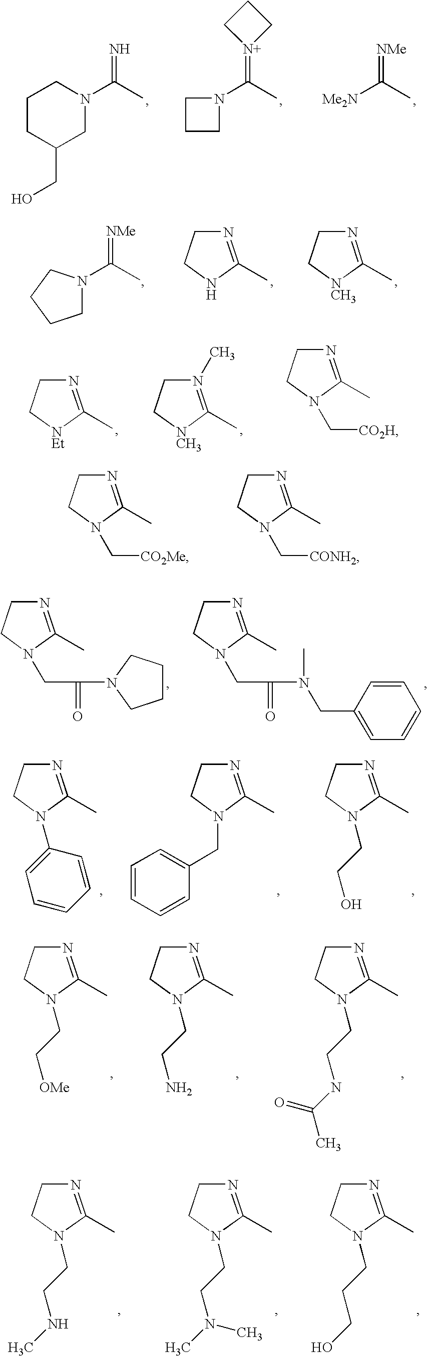

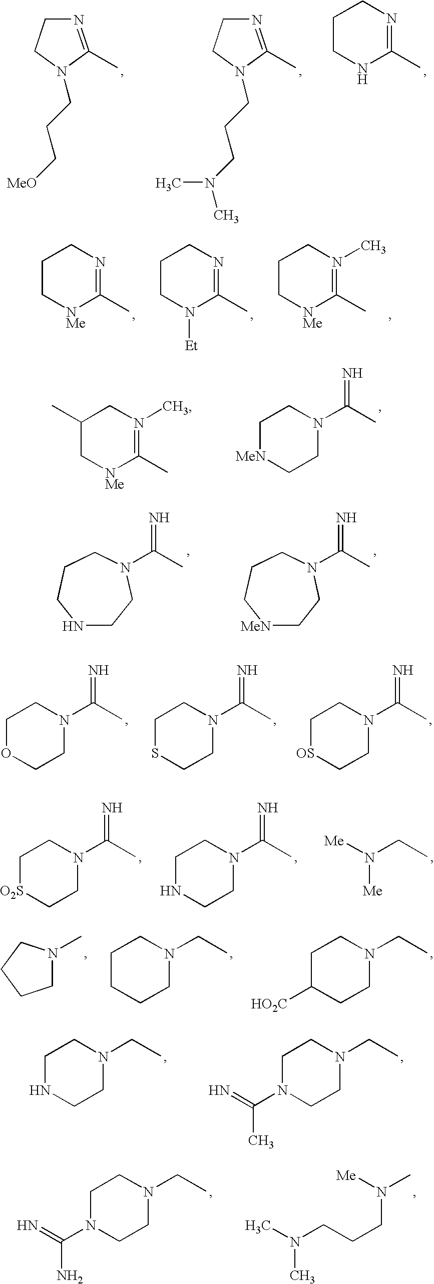

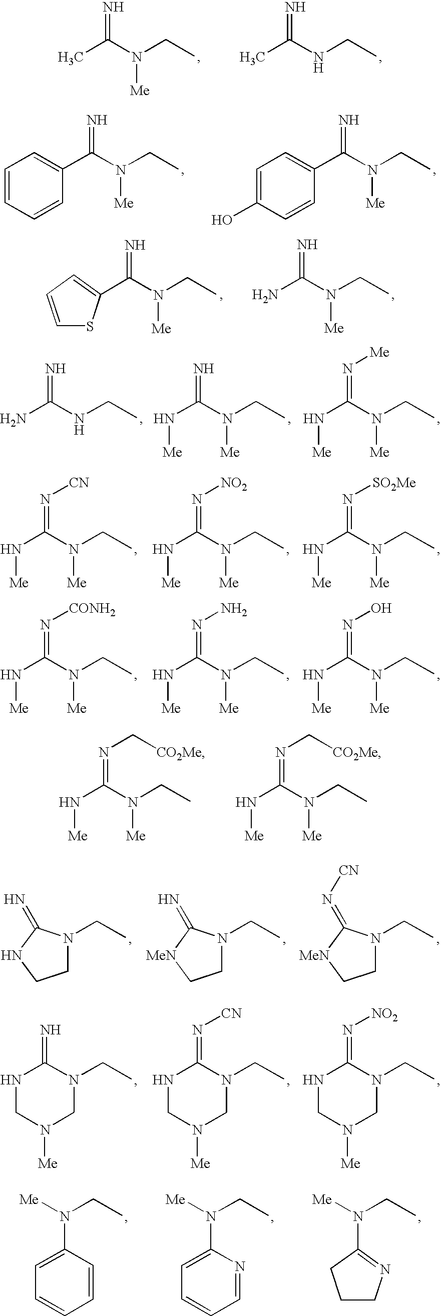

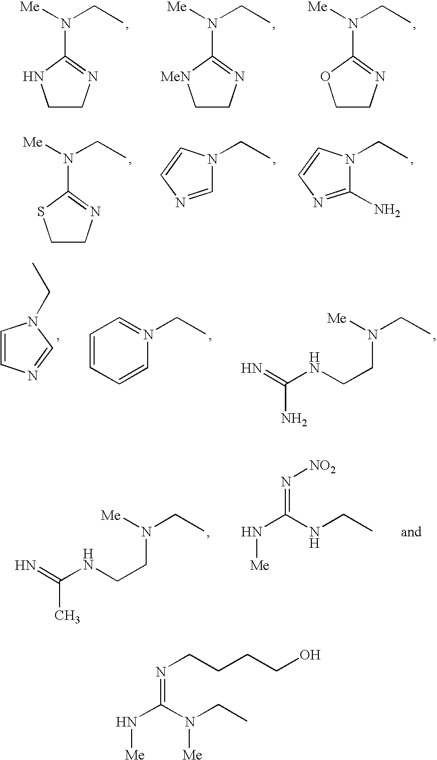

and all pharmaceutically acceptable isomers, salts, hydrates, solvates and prodrug derivatives thereof.

The invention further provides compounds of formula (I) having the following structure:

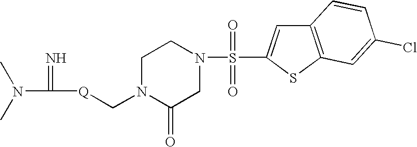

wherein:

Q is a member selected from the group consisting of:

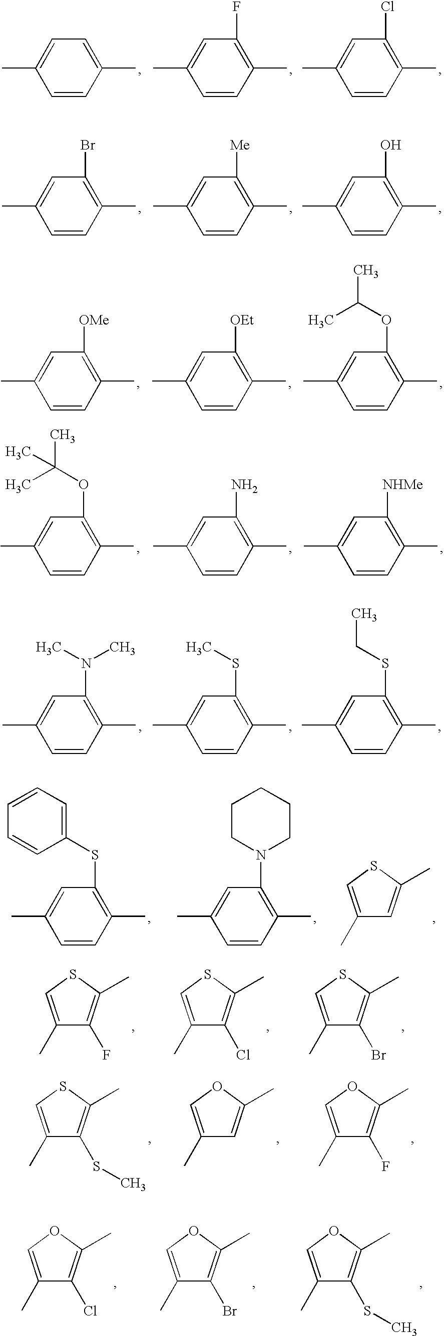

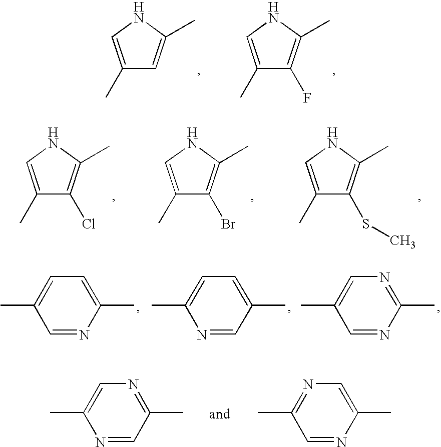

and all pharmaceutically acceptable isomers, salts, hydrates, solvates and prodrug derivatives thereof.

The invention further provides compounds of formula (I) having the following structure:

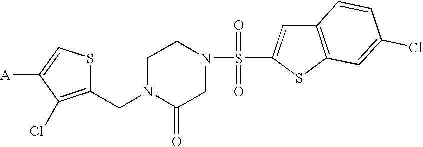

wherein:

A is a member selected from the group consisting of:

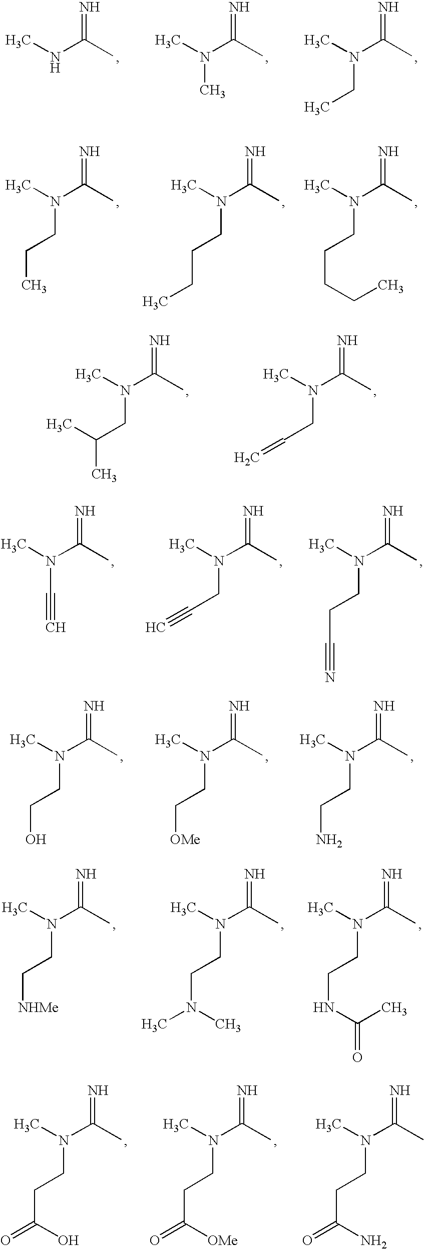

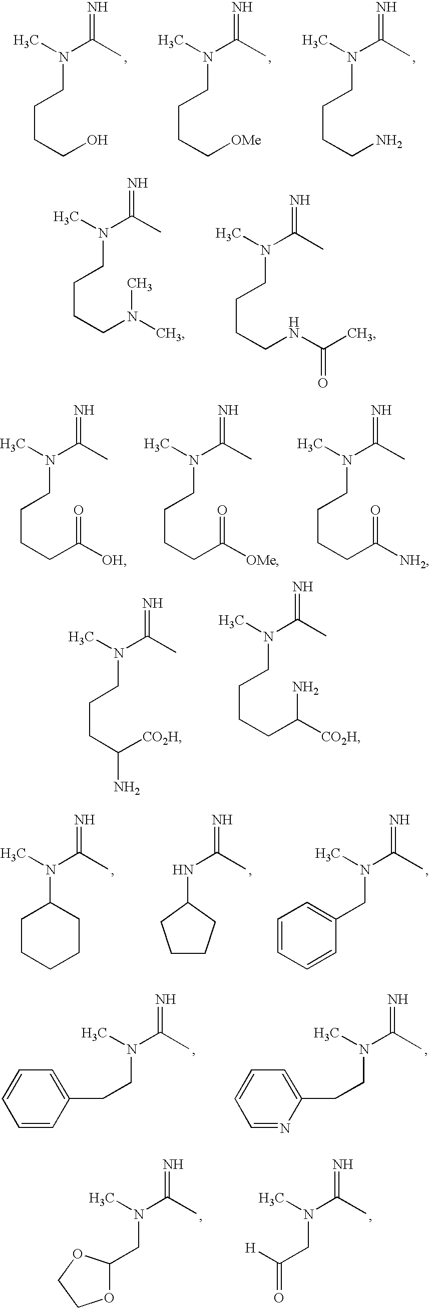

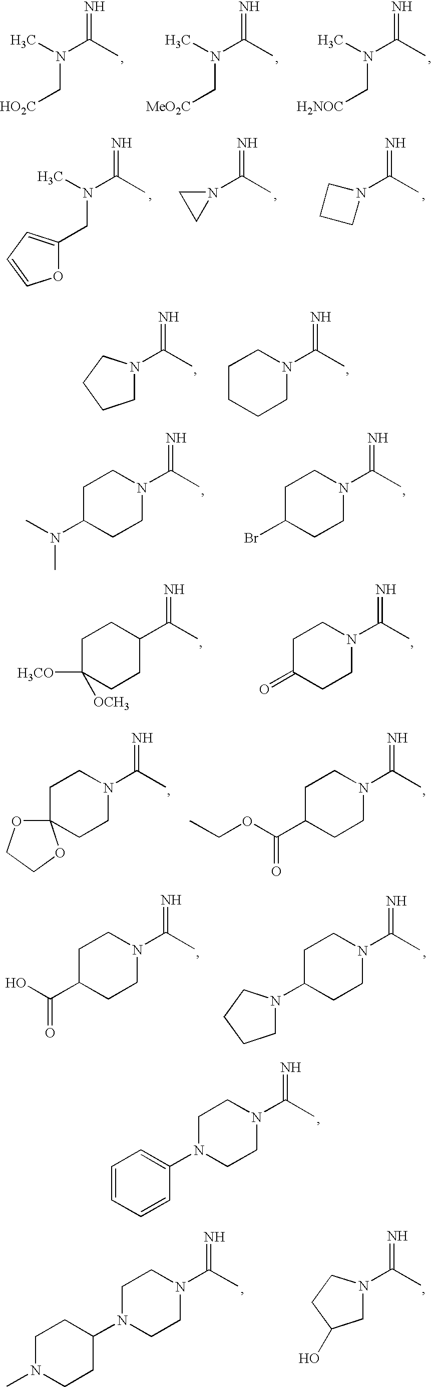

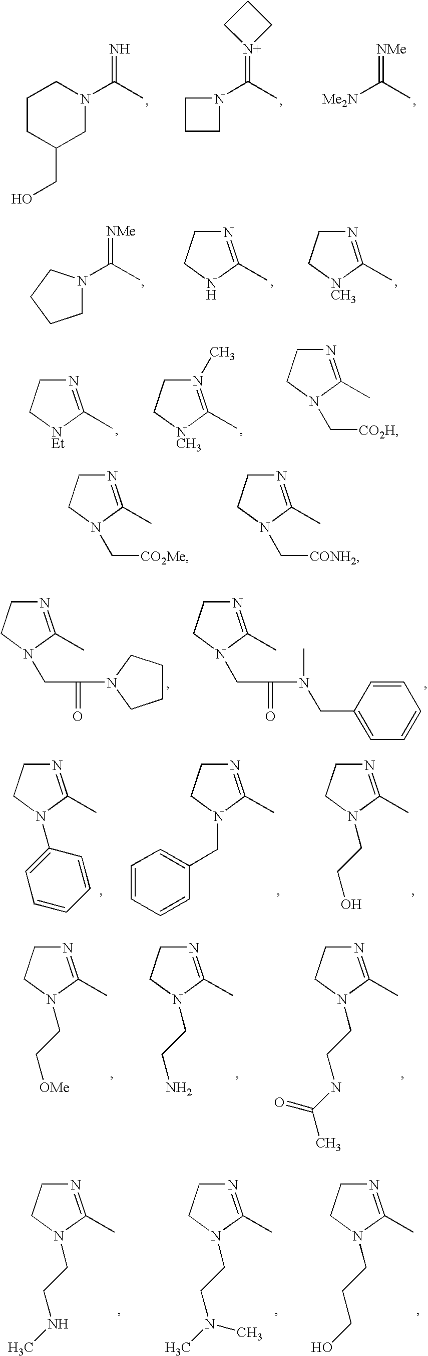

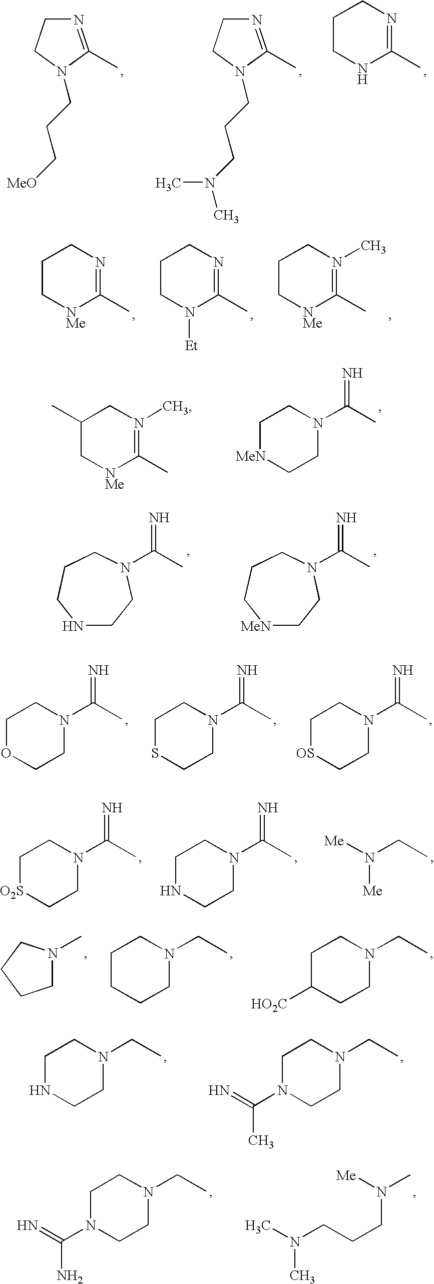

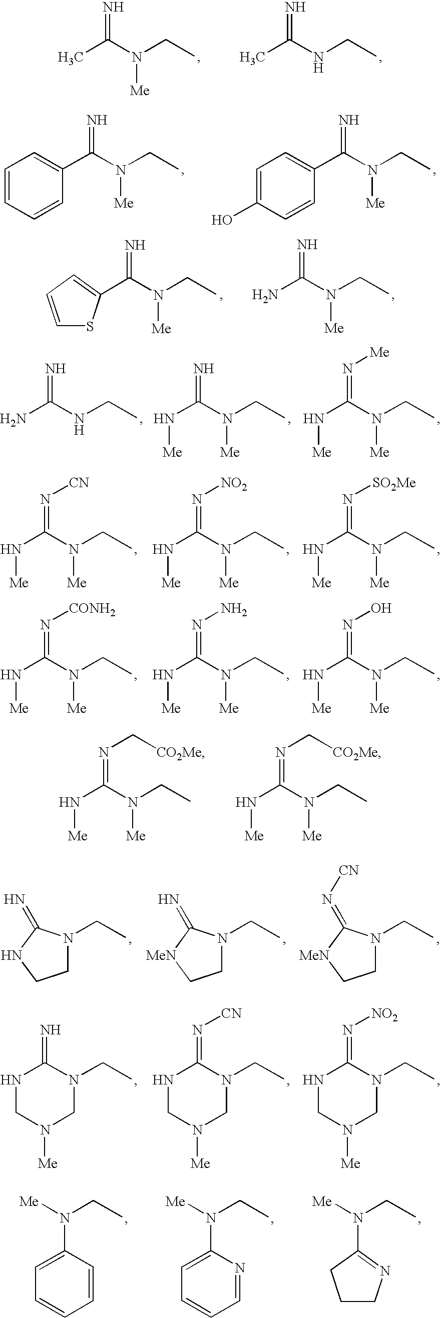

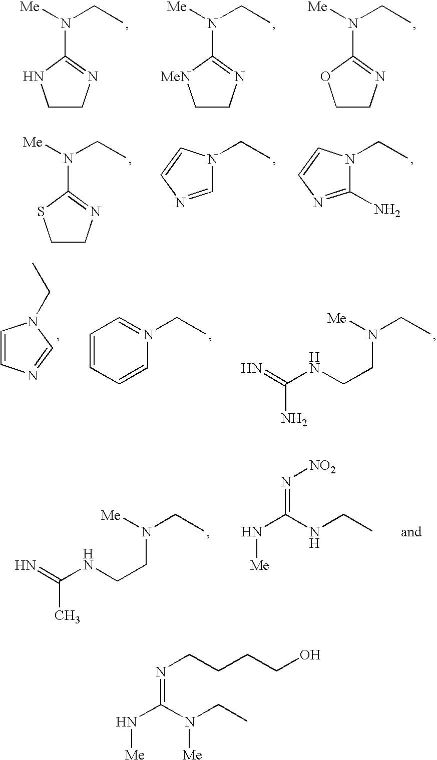

and all pharmaceutically acceptable isomers, salts, hydrates, solvates and prodrug derivatives thereof.

The invention further provides compounds of formula (I) having the following structure:

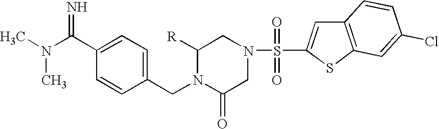

wherein:

R is independently selected from the group consisting of:

H, —CO₂H, —CO₂Me, —CONH₂, —CONHMe, —CONHMe₂, —CON(CH₂)₄, —CON(CH₂)₅, —CH₂OH, —CH₂OMe, —CH₂CO₂H, —CH₂CO₂Me, —CH₂CONH₂, —CH₂CH₂OH, —CH₂CH₂OMe, —CH₂NH₂, —CH₂N(Me)₂, and —CH₃,

and all pharmaceutically acceptable isomers, salts, hydrates, solvates and prodrug derivatives thereof.

The invention further provides compounds of formula (I) having the following structure:

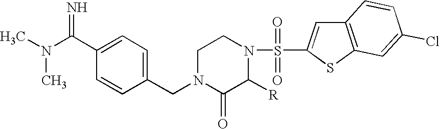

wherein:

R is independently selected from the group consisting of:

H, —CO₂H, —CO₂Me, —CONH₂, —CONHMe, —CONHMe₂, —CON(CH₂)₄, —CON(CH)₅, —CH₂OH, —CH₂OMe, —CH₂CO₂H, —CH₂CO₂Me, —CH₂CONH₂, —CH₂CH₂OH, —CH₂CH₂OMe, —CH₂NH₂, —CH₂N(Me)₂, and —CH₃,

and all pharmaceutically acceptable isomers, salts, hydrates, solvates and prodrug derivatives thereof.

The invention further provides compounds of formula (I) having the following structure:

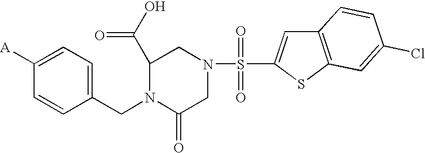

wherein:

A is a member selected from the group consisting of:

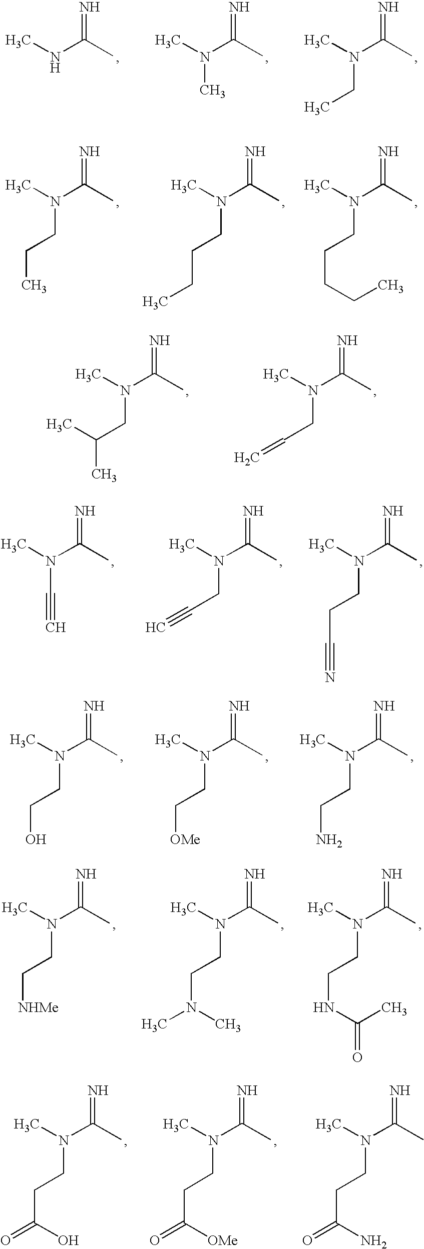

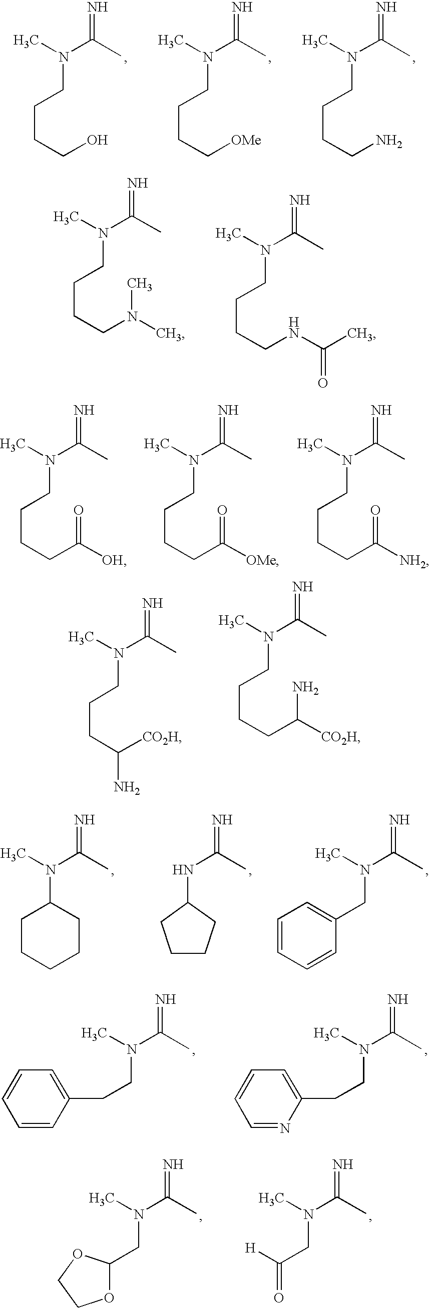

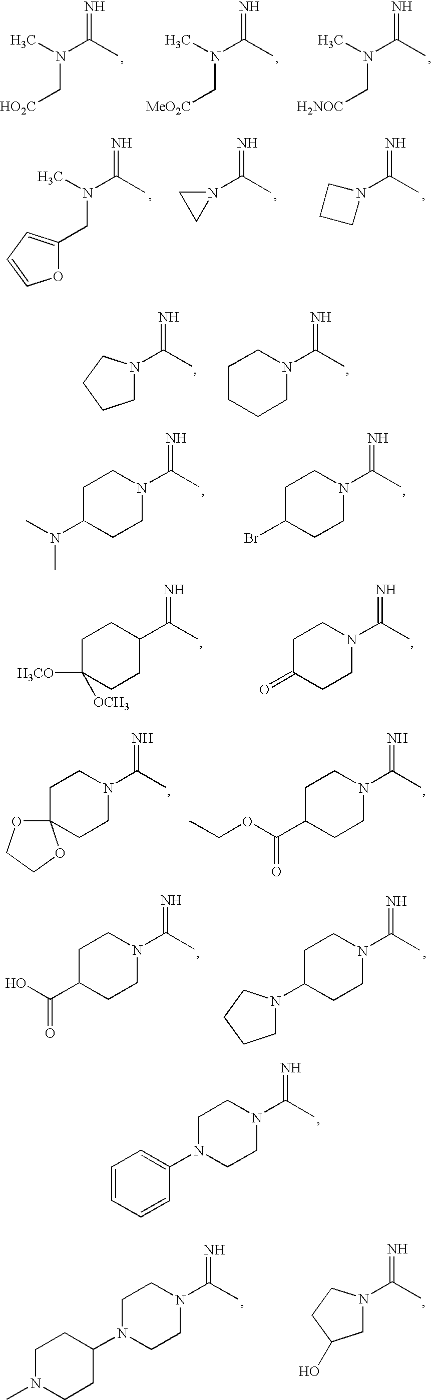

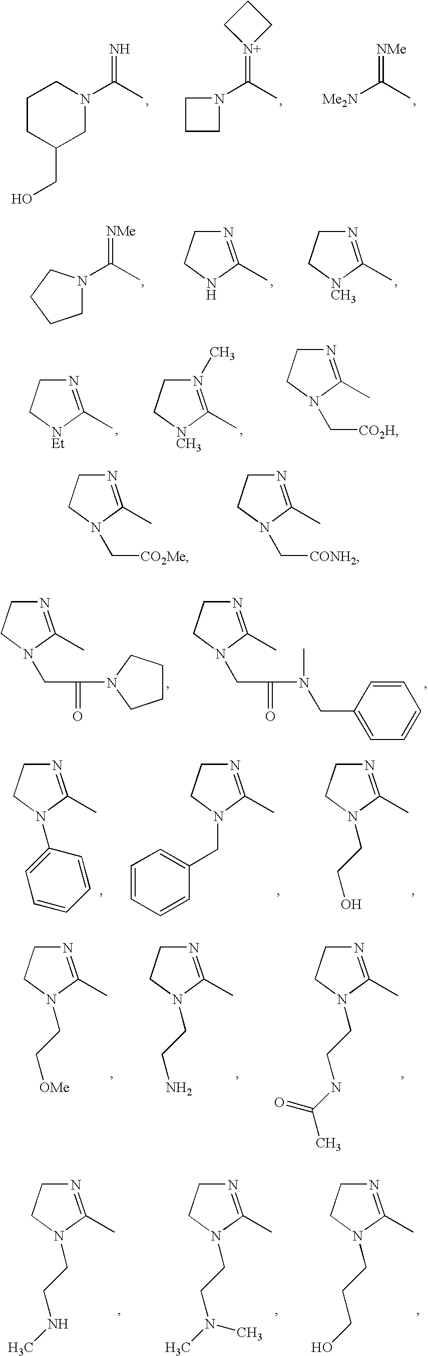

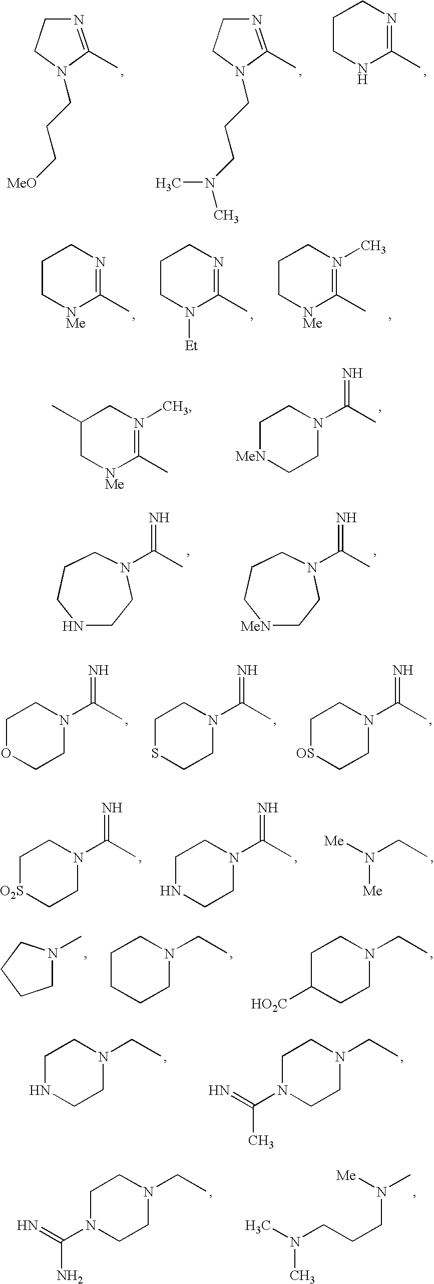

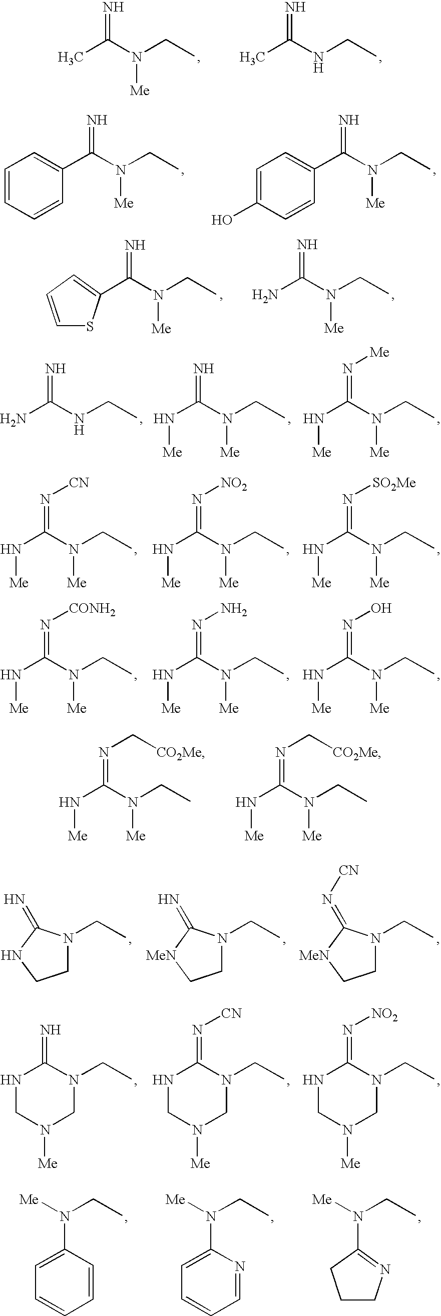

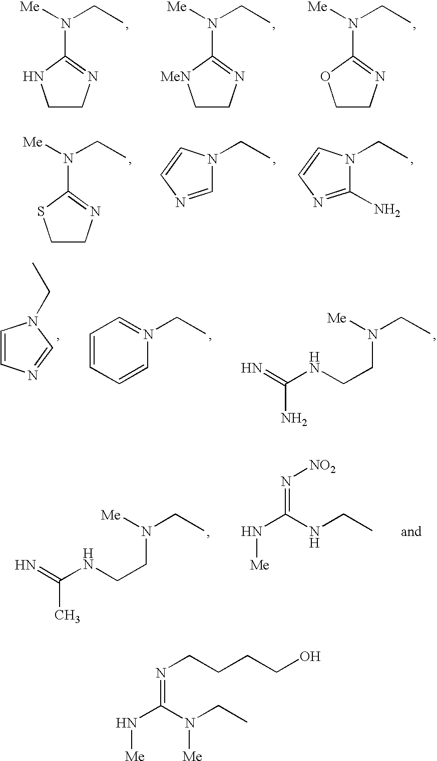

and all pharmaceutically acceptable isomers, salts, hydrates, solvates and prodrug derivatives thereof.

The invention further provides compounds of formula (I) having the following structure:

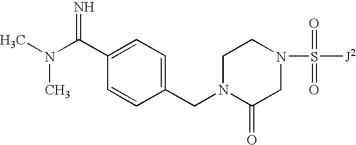

wherein:

J² is a member selected from the group consisting of:

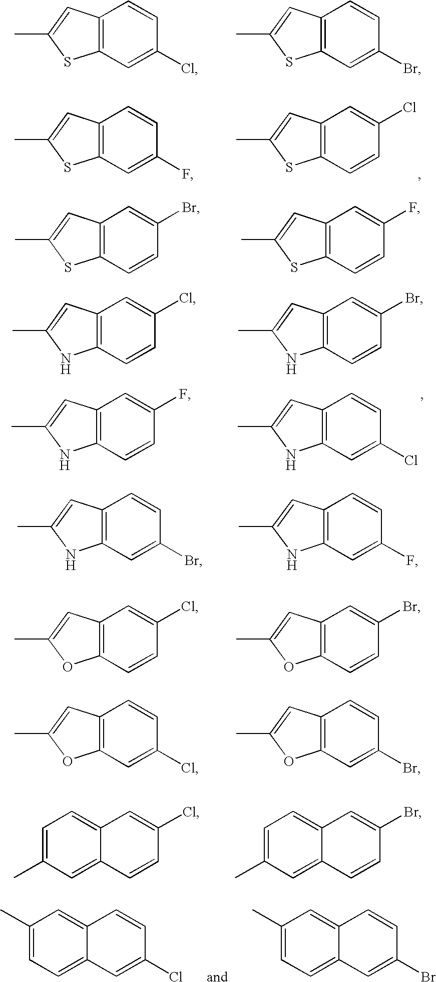

and all pharmaceutically acceptable isomers, salts, hydrates, solvates and prodrug derivatives thereof.

The invention further provides compounds of formula (II) having the following structure:

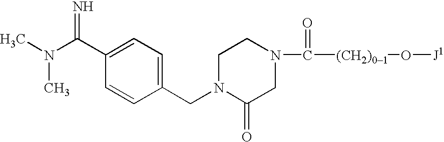

wherein:

J¹ is a member selected from the group consisting of:

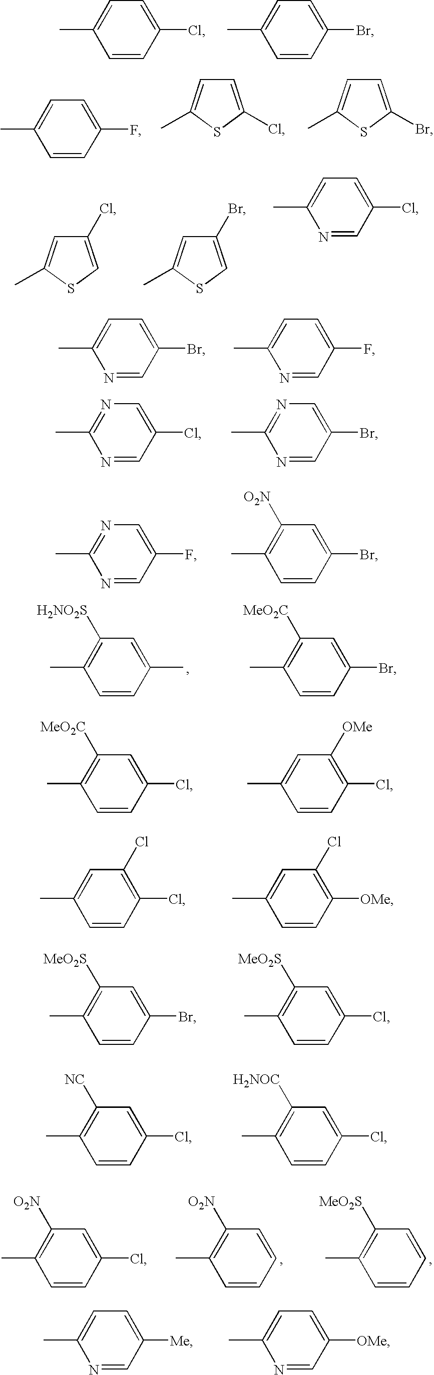

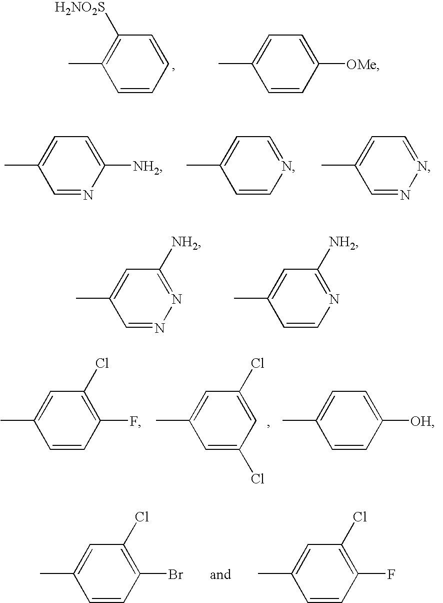

and all pharmaceutically acceptable isomers, salts, hydrates, solvates and prodrug derivatives thereof.

The invention also encompasses all pharmaceutically acceptable isomers, salts, hydrates, solvates and prodrug derivatives of the compounds of the invention as set forth herein. The compounds of the invention can exist in various isomeric and tautomeric forms, and all such forms are meant to be included in the invention, along with pharmaceutically acceptable salts, hydrates, solvates and prodrug derivatives of such isomers and tautomers.

The compounds of the invention may be isolated as the free acid or base or converted to salts of various inorganic and organic acids and bases. Such salts are within the scope of the invention. Non-toxic and physiologically compatible salts are particularly useful although other less desirable salts may have use in the processes of isolation and purification.

A number of methods are useful for the preparation of the salts of the compounds as described above and are known to those skilled in the art. For example, the free acid or free base form of a compound of one of the formulae above can be reacted with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product may be passed over an ion exchange resin to form the desired salt or one salt form of the product may be converted to another using the same general process.

The invention also encompasses prodrug derivatives of the compounds contained herein. The term “prodrug” refers to a pharmacologically inactive derivative of a parent drug molecule that requires biotransformation, either spontaneous or enzymatic, within the organism to release the active drug. Prodrugs are variations or derivatives of the compounds of the invention which have groups cleavable under metabolic conditions. Prodrugs become the compounds of the invention which are pharmaceutically active in vivo, when they undergo solvolysis under physiological conditions or undergo enzymatic degradation. Prodrug compounds of the invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of the invention may be combined with other features herein taught to enhance bioavailability.

The compounds of the present invention may also be used alone or in combination or in combination with other therapeutic or diagnostic agents. In certain preferred embodiments, the compounds of the invention may be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds of the present invention may act in a synergistic fashion to prevent reocclusion following a successful thrombolytic therapy and/or reduce the time to reperfusion. These compounds may also allow for reduced doses of the thrombolytic agents to be used and therefore minimize potential hemorrhagic side-effects. The compounds of the invention can be utilized in vivo, ordinarily in mammals such as primates (e.g. humans), sheep, horses, cattle, pigs, dogs, cats, rats and mice, or in vitro.

The biological properties of the compounds of the present invention can be readily characterized by methods that are well known in the art, for example by the in vitro protease activity assays and iil vivo studies to evaluate antithrombotic efficacy, and effects on hemostasis and hematological parameters, such as are illustrated in the examples.

Diagnostic applications of the compounds of the invention will typically utilize formulations in the form of solutions or suspensions. In the management of thrombotic disorders the compounds of the invention may be utilized in compositions such as tablets, capsules or elixirs for oral administration, suppositories, sterile solutions or suspensions or injectable administration, and the like, or incorporated into shaped articles. Subjects in need of treatment (typically mammalian) using the compounds of the invention can be administered dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Preparation of Compounds

The compounds of the present invention may be synthesized by standard organic chemical synthetic methods as described and referenced in standard textbooks. These methods are well known in the art. See, e.g., Morrison and Boyd, “Organic Chemistry”, Allyn and Bacon, Inc., Boston, 1959, et seq.

Starting materials used in any of these methods are commercially available from chemical vendors such as Aldrich, Sigma, Nova Biochemicals, Bachem Biosciences, and the like, or may be readily synthesized by known procedures.

Reactions are carried out in standard laboratory glassware and reaction vessels under reaction conditions of standard temperature and pressure, except where otherwise indicated.

During the synthesis of these compounds, the functional groups of the substituents are optionally protected by blocking groups to prevent cross reaction during coupling procedures. Examples of suitable blocking groups and their use are described in “The Peptides: Analysis, Synthesis, Biology”, Academic Press, Vol. 3 (Gross, et al., Eds., 1981) and Vol. 9 (1987), the disclosures of which are incorporated herein by reference.

Non-limiting exemplary synthesis schemes are outlined directly below, and specific steps are described in the Examples. The reaction products are isolated and purified by conventional methods, typically by solvent extraction into a compatible solvent. The products may be further purified by column chromatography or other appropriate methods.

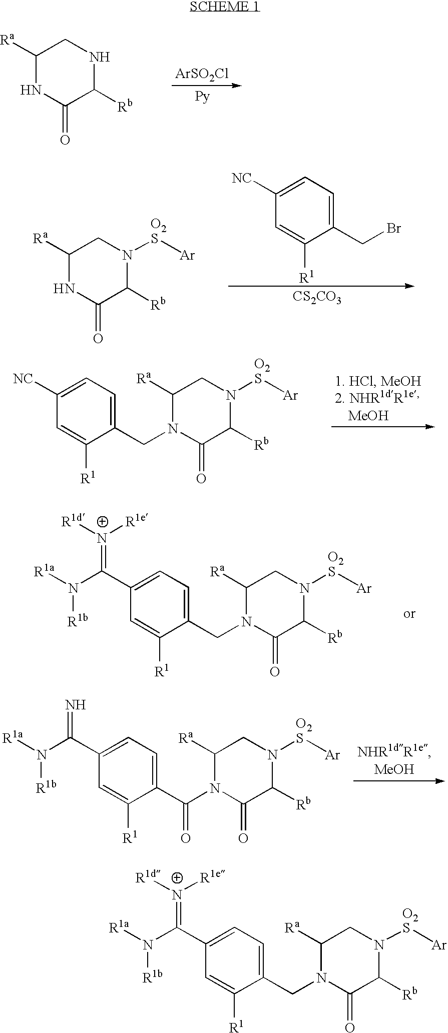

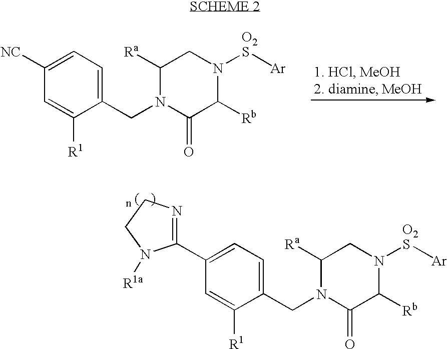

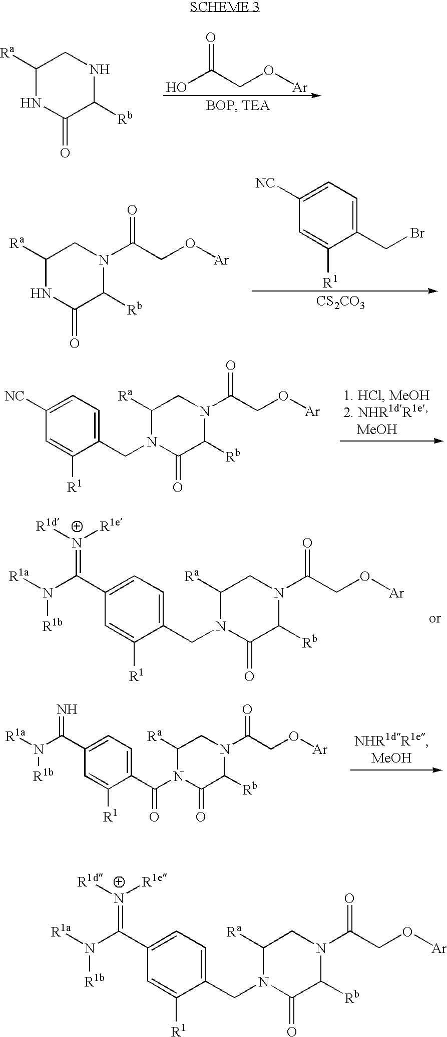

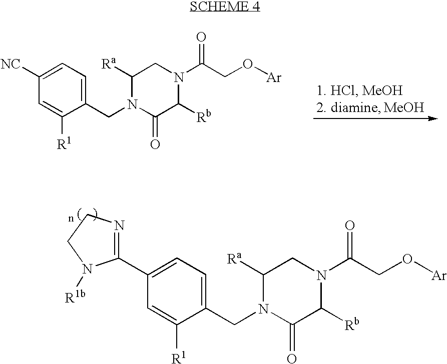

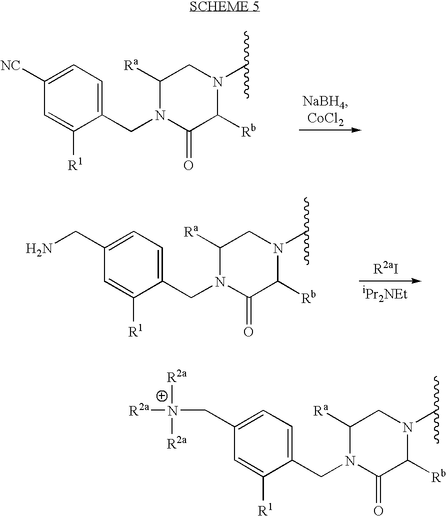

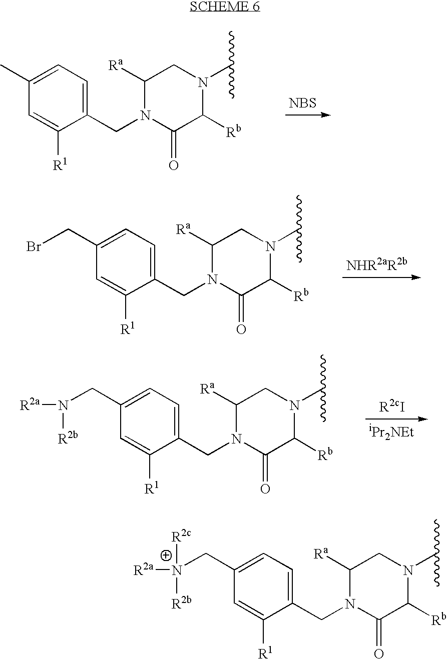

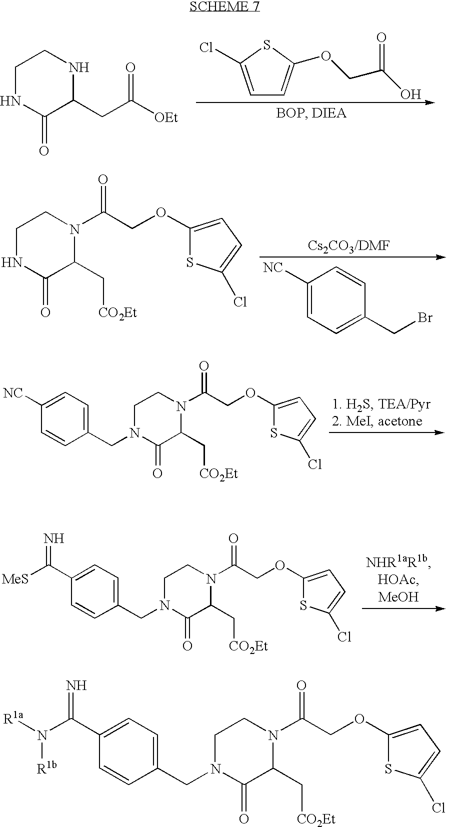

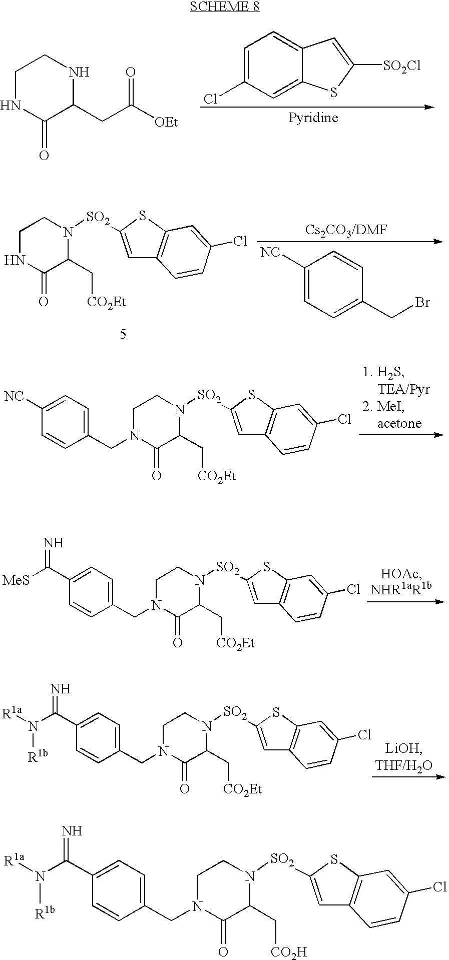

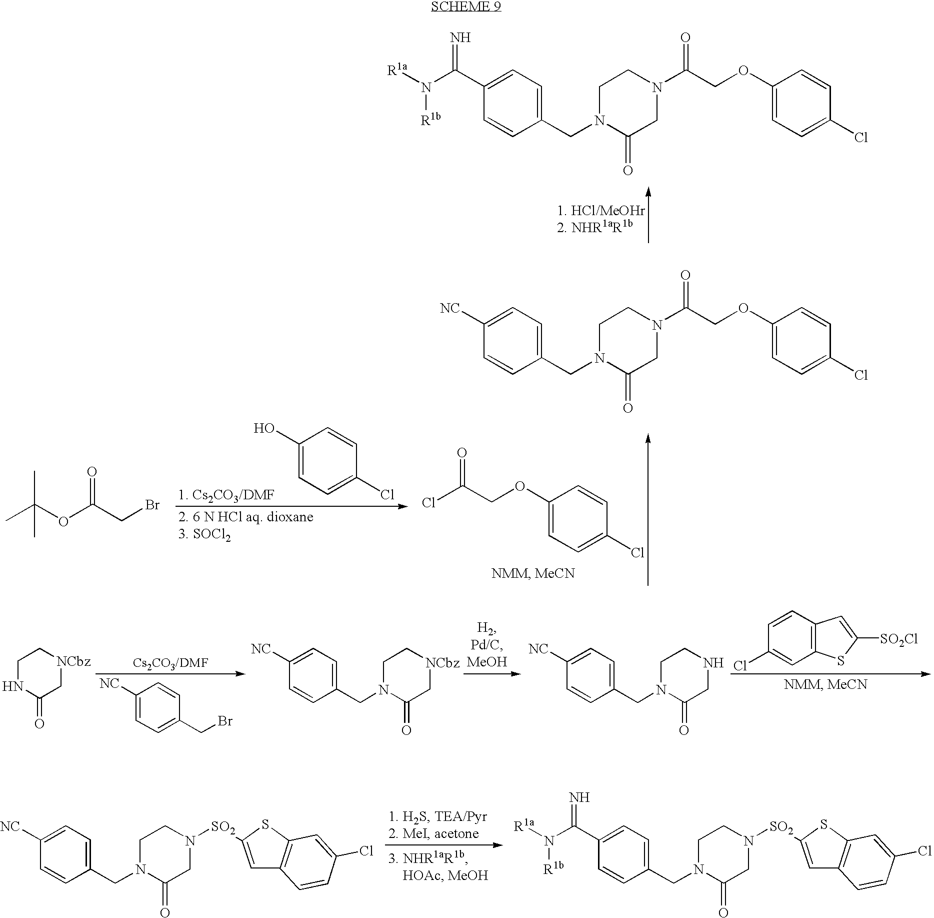

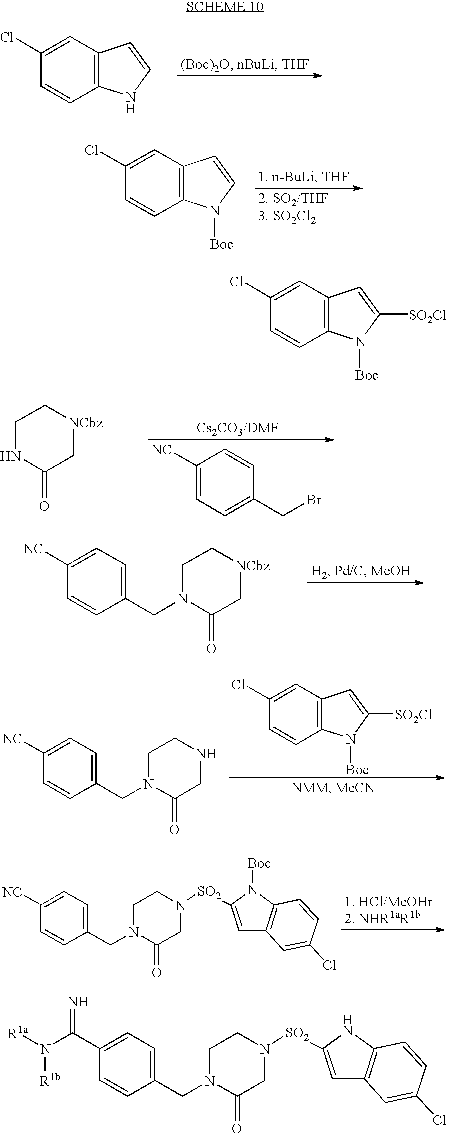

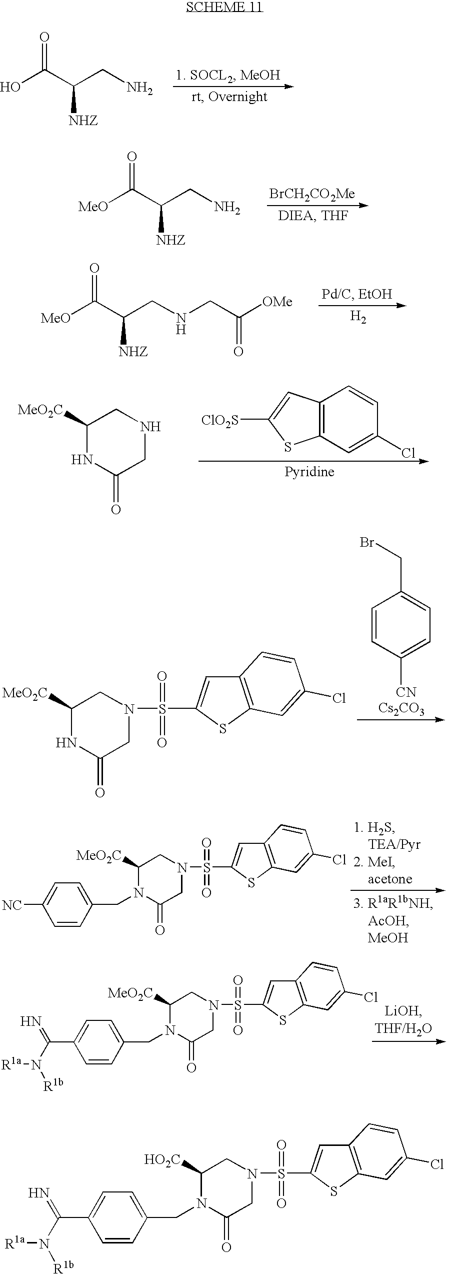

The following examples are non-limiting embodiments of the present invention, which were made utilizing a method as generally shown in reaction Schemes 1-11 above or by a similar procedure as would be understood by one of skill in the art.

### EXAMPLES

### Example 1

### Ethyl 2-[4-({4-[(dimethylamino)iminomethyl]phenyl}methyl)-1-[2-(5-chloro(2-thienyloxy))acetyl]-3-oxopiperazin-2-yl]acetate

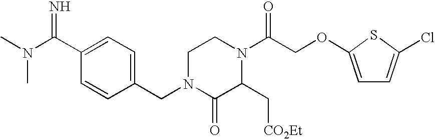

Part 1. To a solution of ethyl 3-oxopiperazine-2-acetate (482.7 mg, 2.59 mmol) in DMF (8 mL) at room temperature was added 5-chloro-2-thienyloxyacetic acid (Ewing, W. R., WO 0032590, 2000) (415 mg, 2.16 mmol), DIEA (0.75 mL, 4.32 mmol), and BOP (1.15 g, 2.59 mmol). The solution was stirred at room temperature for 24 hours. The reaction mixture was diluted in EtOAc and water. The organic layer was washed with sat. NaHCO₃ and sat. NaCl, dried over MgSO₄, filtered and concentrated in vacuo to yield ethyl 2-{1-[2-(5-chloro(2-thienyloxy))acetyl]-3-oxopiperazin-2-yl}acetate (0.56 g, 72% yield). MS found for (M+H)+: 361.1.

Part 2. To a solution ethyl 2-{1-[2-(5-chloro(2-thienyloxy))acetyl]-3-oxopiperazin-2-yl}acetate (560 mg, 1.56 mmol) and -bromo-p-tolunitrile (305 mg,1.56 mmol) in 5 mL of DMF was added Cs₂CO₃ (762 mg, 2.34 mmol). After stirring at rt for 24 h, the mixture was diluted with EtOAc and washed with H₂O. The organic layer was dried over MgSO₄, filtered and evaporated in vacuo. Flash chromatography on silica gel (10% EtOAc in DCM) gave ethyl 2-{1-[2-(5-chloro(2-thienyloxy))acetyl]-4-[(4-cyanophenyl)methyl]-3-oxopiperazin-2-yl}acetate (320 mg, 43%). MS found for C₁₃H₉N₂O₃ (M+H)⁺: 476.

Part 3. A stream of H₂S (g) was bubbled through a solution of ethyl 2-{1-[2-(5-chloro(2-thienyloxy))acetyl]-4-[(4-cyanophenyl)methyl]-3-oxopiperazin-2-yl}acetate (320 mg, 0.67 mmol) in 4.5 mL pyridine and 0.5 mL NEt₃ until saturation. The mixture was stirred at rt for 4-5 hr and evaporated. The resulting residue was treated with MeI (0.42 mL, 6.74 mmol) in 5 mL acetone at reflux temperature for 1 hr and concentrated to dryness. The resulting residue was treated with a mixture of dimethylamine (1.68 mL of 2 M solution in THF, 3.37 mmol) in acetic acid (0.29 mL, 5.1 mmol) and 4 mL methanol under reflux for 30 min. The solvent was removed at reduced pressure and the crude product was purified by HPLC (C18 reversed phase) eluting with 0.1% TFA in H₂O/CH₃CN to give Ethyl 2-[4-({4-[(dimethylamino)iminomethyl]phenyl}methyl)-1-[2-(5-chloro(2-thienyloxy))acetyl]-3-oxopiperazin-2-yl]acetate. MS found: (M+H)⁺: 521.1.

### Example 2

### Ethyl 2-(1-[2-5-chloro(2-thienyloxy))acetyl]-4-{[4-(iminopyrrolidinylmethyl) phenyl]methyl}-3-oxopiperazin-2-yl)acetate

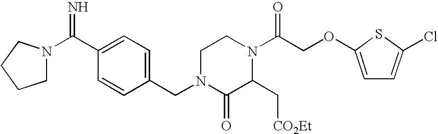

The titled compound was synthesized using a similar procedure to that described in Example 1 using pyrrolidine instead of dimethylamine in Part 3. MS found: (M+H)+: 580.1.

### Example 3

### 2-[4-({4-[(Dimethylamino)iminomethyl]phenyl}methyl)-1-[2-(5-chloro(2-thienyloxy))acetyl]-3-oxopiperazin-2-yl]acetic acid

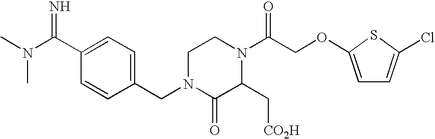

A solution of ethyl 2-[4-({4-[(dimethylamino)iminomethyl]phenyl}methyl)-1-[2-(5-chloro(2-thienyloxy))acetyl]-3-oxopiperazin-2-yl]acetate (40 mg, 0.077 mmol) in 2 mL of methanol and 2 mL of H₂O was treated with LiOH (6.46 mg, 0.154 mmol) at rt for 2 h. Methanol was evaporated, and the H₂O layer was acidified with 1N HCl until pH˜1-2. The H₂O layer was extracted with EtOAc. The organic layer was dried over MgSO₄, filtered and evaporated in vacuo and the crude product was purified by HPLC (C18 reversed phase) eluting with 0.1% TFA in H₂O/CH₃CN to give 2-[4-({4-[(Dimethylamino)iminomethyl]phenyl}methyl)-1-[2-(5-chloro(2-thienyloxy))acetyl]-3-oxopiperazin-2-yl]acetic acid. MS found: (M+H)⁺: 493.1.

### Example 4

### 2-(1-[2-(5-Chloro(2-thienyloxy))acetyl]-4-{[4-(iminopyrrolidinylmethyl)phenyl]methyl}-3-oxopiperazin-2-yl)acetic acid

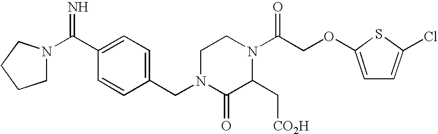

The titled compound was synthesized using a similar procedure to that described in Example 3 using ethyl 2-(1-[2-(5-chloro(2-thienyloxy))acetyl]-4-{[4-(iminopyrrolidinylmethyl)phenyl]methyl}-3-oxopiperazin-2-yl)acetate. MS found: (M+H)+: 579.1.

### Example 5

### Ethyl 2-[4-({4-[(dimethylamino)iminomethyl]phenyl}methyl)-1-[(6-chlorobenzo [b]thiophen-2-yl)sulfonyl]-3-oxopiperazin-2-yl]acetate

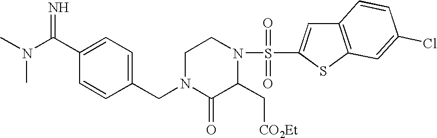

Part 1. To a solution of ethyl 3-oxopiperazine-2-acetate (1.12 g, 6 mmol) in pyridine (15 mL) at rt, was added 6-chlorobenzo[b]thiophene-2-sulfonyl chloride (Ewing, W. R., WO 9937304) (1.33 g, 5 mmol). The mixture was stirred at rt overnignt and evaporated in vacuo. The residue was dissolved in EtOAc and H₂O. The organic layer was washed with water, 1N HCl, sat. NaHCO₃, sat. NaCl, dried over Na₂SO₄ and evaporated to give ethyl 2-{1-[(6-chlorobenzo[b]thiophen-2-yloxy)sulfinyl]-3-oxopiperazin-2-yl}acetate as a solid (1.43 g, 69%% yield). MS found (M+H)+: 417.1.

Part 2. To a solution of ethyl 2-{1-[(6-chlorobenzo[b]thiophen-2-yloxy)sulfinyl]-3-oxopiperazin-2-yl}acetate (1.04 g, 2.5 mmol) and -bromo-p-tolunitrile (588mg, 3 mmol) in 15 mL of DMF was added Cs₂CO₃ (1.22 g, 3.75 mmol). After stirring at rt for 24 h, the mixture was diluted with EtOAc and washed with H₂O. The organic layer was dried over MgSO₄, filtered and evaporated in vacuo. Flash chromatography on silica gel (40% EtOAc in hexane) gave ethyl 2-{1-[(6-chlorobenzo[b]thiophen-2-yloxy)sulfinyl]-4-[(4-cyanophenyl)methyl]-3-oxopiperazin-2-yl}acetate (320 mg, 43%). MS found (M+H)⁺: 532.1.

Part 3. A stream of H₂S (g) was bubbled through a solution of ethyl 2-{1-[(6-chlorobenzo[b]thiophen-2-yloxy)sulfinyl]-4-[(4-cyanophenyl)methyl]-3-oxopiperazin-2-yl}acetate (524 mg, 0.985 mmol) in 9 mL pyridine and 1 mL NEt₃ until saturation. The mixture was stirred at rt for 24 hr and evaporated. The resulting residue was treated with MeI (0.613 mL, 9.85 mmol) in 5 mL acetone at reflux temperature for 1 hr and concentrated to dryness. The resulting residue was treated with a mixture of dimethylamine (2.48 mL of 2 M solution in THF, 4.92 mmol) in acetic acid (0.42 mL, 7.38 mmol) and 16 mL methanol under reflux for 30 min. The solvent was removed at reduced pressure and the crude product was purified by HPLC (C18 reversed phase) eluting with 0.1% TFA in H₂O/CH₃CN to give ethyl 2-[4-({4-[(dimethylamino)iminomethyl]phenyl}methyl)-1-[(6-chlorobenzo[b]thiophen-2-yl)sulfonyl]-3-oxopiperazin-2-yl]acetate. MS found: (M+H)⁺: 577.1.

### Example 6

### Ethyl 2-(1-[(6-chlorobenzo[b]thiophen-2-yl)sulfonyl]-4-{[4-(iminopyrrolidinylmethyl)phenyl]methyl}-3-oxopiperazin-2-yl)acetate

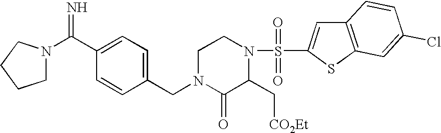

The titled compound was synthesized using a similar procedure to that described in Example 5 using pyrrolidine instead of dimethylamine in Part 3. MS found: (M+H)+: 604.1.

### Example 7

### Ethyl 2-(1-[(6-chlorobenzo[b]thiophen-2-yl)sulfonyl]-4-{[4-(1-methyl(2-imidazolin-2-yl))phenyl]methyl}-3-oxopiperazin-2-yl)acetate

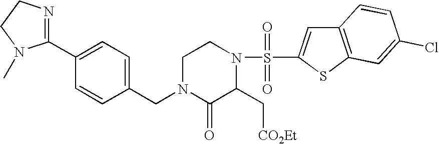

The titled compound was synthesized using a similar procedure to that described in Example 5 using N-methylethylenediamine instead of dimethylamine in Part 3. MS found: (M+H)+: 590.1.

### Example 8

### 2-[4-({4-[(dimethylamino)iminomethyl]phenyl}methyl)-1-[(6-chlorobenzo[b]thiophen-2-yl)sulfonyl]-3-oxopiperazin-2-yl]acetic acid

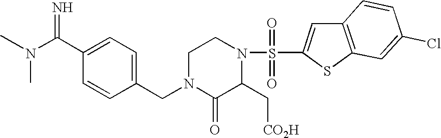

A solution of ethyl 2-[4-({4-[(dimethylamino)iminomethyl]phenyl}methyl)-1-[(6-chlorobenzo[b]thiophen-2-yl)sulfonyl]-3-oxopiperazin-2-yl]acetate (40 mg, 0.07 mmol) in 2 mL of methanol and 2 mL of H₂O was treated with LiOH (5.8 mg, 0.14 mmol) at rt for 2 h. Methanol was evaporated, and the H₂O layer was acidified with 1N HCl until pH˜1-2. The H₂O layer was extracted with EtOAc. The organic layer was dried over MgSO₄, filtered and evaporated in vacuo and the crude product was purified by HPLC (C18 reversed phase) eluting with 0.1% TFA in H₂O/CH₃CN to give 2-[4-({4-[(dimethylamino)iminomethyl]phenyl}methyl)-1-[(6-chlorobenzo[b]thiophen-2-yl)sulfonyl]-3-oxopiperazin-2-yl]acetic acid. Mass found: (M+H)⁺: 550.

### Example 9

### 2-(1-[(6-Chlorobenzo[b]thiophen-2-yl)sulfonyl]-4-{[4-(iminopyrrolidinylmethyl) phenyl]methyl}-3-oxopiperazin-2-yl)acetic acid

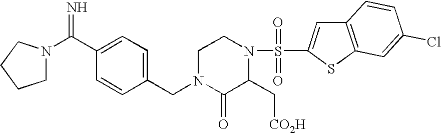

The titled compound was synthesized using a similar procedure to that described in Example 8 using Ethyl 2-(1-[(6-chlorobenzo[b]thiophen-2-yl)sulfonyl]-4-{[4-(iminopyrrolidinylmethyl)phenyl]methyl}-3-oxopiperazin-2-yl)acetate. MS found: (M+H)+: 576.1.

### Example 10

### 2-(1-[(6-chlorobenzo[b]thiophen-2-yl)sulfonyl]-4-{[4-(1-methyl(2-imidazolin-2-yl))phenyl]methyl}-3-oxopiperazin-2-yl)acetic acid

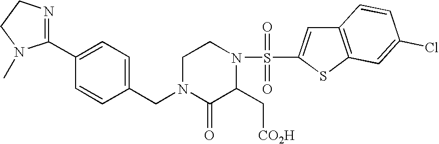

The titled compound was synthesized using a similar procedure to that described in Example 8 using ethyl 2-(1-[(6-chlorobenzo[b]thiophen-2-yl)sulfonyl]-4-{[4-(1-methyl(2-imidazolin-2-yl))phenyl]methyl}-3-oxopiperazin-2-yl)acetate. MS found: (M+H)+: 562.1.

### Example 11

### 1-({4-[(Dimethylamino)iminomethyl]phenyl}methyl)-4-[2-(4-chlorophenoxy)acetyl]piperazin-2-one

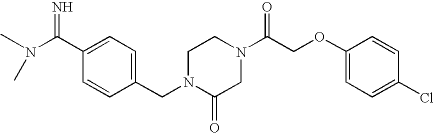

Part 1. A mixture of 4-benzyloxycarbonylpiperazin-2-one (3 g, 12.82 mmol), -bromo-p-tolunitrile (6.71 g, 19.23 mmol), and Cs₂CO₃ (12.53 g, 38.46 mmol) in DMF (10 mL) was stirred at 50° C. for 24 hours. The solid was filtered and the filtrate was concentrated. The residue was diluted with EtOAc, washed with sat. NaCl (2×), dried and evaporated. The crude material was subjected to silica gel chromatography to afford phenylmethyl 4-[(4-cyanophenyl)methyl]-3-oxopiperazinecarboxylate (3.5 g, 78%). MS found: (M+H)+: 350.1.

Part 2. A mixture of phenylmethyl 4-[(4-cyanophenyl)methyl]-3-oxopiperazinecarboxylate (1 g, 2.87 mmol) and 10% Pd/C (160 mg) in MeOH (10 mL) was stirred under balloon H₂ (1 atm) for 3 hrs. The Pd/C was filtered off through a celite bed. The filtrate was evaporated to give 4-[(2-oxopiperazinyl)methyl]benzenecarbonitrile (600 mg, 97%). MS found: (M+H)+: 216.1.

Part 3. A mixture of 4-chlorophenol (3 g; 23.44 mmol), tert-butyl bromoacetate (6.82 g, 35.16 mmol), and Cs₂CO₃ (23.04 g, 70.72 mmol) in DMF (50 mL) was stirred at 50° C. for 24 hours. The solid was filtered and the filtrate was concentrated. The residue was diluted with EtOAc and water, washed with sat. NaCl (2×), dried and evaporated. The crude material was subjected to silica gel chromatography to afford tert-butyl 2-(4-chlorophenoxy)acetate (5.2 g, 92%). MS found: (M+H)+: 243.1.

Part 4. tert-butyl 2-(4-chlorophenoxy)acetate (2 g, 8.26 mmol) was dissolved in dioxane. 6 M HCl (10 mL) was added. The mixture was stirred at rt for 1 hr and diluted with EtOAc (20 mL) and water (10 ML). The organic layer was separated and extracted into 0.5 N NaOH (10 mL). H₂O layer was washed with EtOAc and acidified with 1N HCl until pH˜1-2. The H₂O layer was extracted with EtOAc. The organic layer was dried over MgSO₄, filtered and evaporated in vacuo. Thionyl chloride (5 mL) was added to the residue and the mixture was refluxed for 2 hrs, evaporated in vacuo to give 2-(4-chlorophenoxy)acetyl chloride (1.34 g, 80% yield). MS found: (M+H)⁺: 205.1.

Part 5. To a solution of 4-[(2-oxopiperazinyl)methyl]benzenecarbonitrile (600 mg, 2.79 mmol) and 2-(4-chlorophenoxy)acetyl chloride (683 mg, 3.35 mmol) in CH₃CN (10 mL) at rt, was added N-methylmorpholine (1.13 g, 11.16 mmol). The mixture was stirred at rt for 12 hrs. The solvent was evaporated. The residue was diluted with EtOAc, washed with 1 N HCl, sat. NaHCO₃, sat. NaCl, dried with Na₂SO₄ and evaporated to afford 4-({4-[2-(4-chlorophenoxy)acetyl]-2-oxopiperazinyl}methyl)benzenecarbonitrile (750 mg, 70% yield). MS found: (M+H)⁺: 394.1.

Part 6. A stream of HCl (g) was bubbled through a solution of part 5 (400 mg, 1.04 mmol) in MeOH (10 mL) at 0° C. for 10 min. The mixture was stirred at rt for 4 hr and evaporated in vacuo. The resulting residue was dissolved in MeOH (30 mL), treated with dimethylamine (2.61 mL of 2 M solution in THF, 5.22 mmol) at reflux temperature for 1 hr and concentrated to dryness. The crude product was purified by HPLC (C18 reversed phase) eluting with 0.1% TFA in H₂O/CH₃CN to give -({4-[(Dimethylamino)iminomethyl]phenyl}methyl)-4-[2-(4-chlorophenoxy)acetyl]piperazin-2-one. MS found: (M+H)⁺: 429.1.

### Example 12

### 4-[2-(4-Chlorophenoxy)acetyl]-1-{[4-(1-methyl(2-imidazolin-2-yl))phenyl]methyl}piperazin-2-one

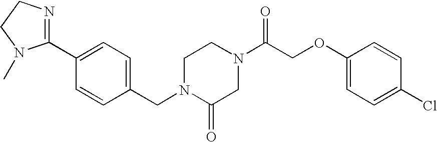

The titled compound was synthesized using a similar procedure to that described in Example 11 using N-methylethylenediamine instead of dimethylamine in part 6. MS found: (M+H)+: 441.1.

### Example 13

### 1-{[4-(Azetidinyliminomethyl)phenyl]methyl}4-[2-(4-chlorophenoxy)acetyl]piperazin-2-one

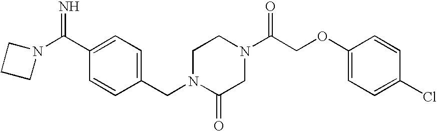

The titled compound was synthesized using a similar procedure to that described in Example 11 using azetidine instead of dimethylamine in Part 6. MS found: (M+H)+: 441.1.

### Example 14

### 4-[2-(4-Chlorophenoxy)acetyl]-1-{[4-(iminopyrrolidinylmethyl) phenyl]methyl}piperazin-2-one

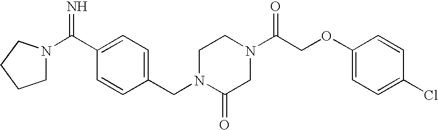

The titled compound was synthesized using a similar procedure to that described in Example 11 using pyrrolidine instead of dimethylamine in Part 6. MS found: (M+H)+: 455.1.

### Example 15

### 4-[2-(4-Chlorophenoxy)acetyl]-1-{[4-(iminopiperidylmethyl)phenyl]methyl}piperazin-2-one

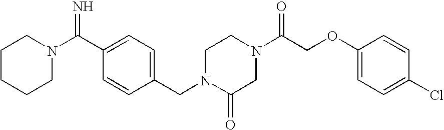

The titled compound was synthesized using a similar procedure to that described in Example 11 using piperidine instead of dimethylamine in Part 6. MS found: (M+H)+: 469.1.

### Example 16

### 1-({4-[(Dimethylamino)iminomethyl]phenyl}methyl)-4-[(6-chlorobenzo[b]thiophen-2-yl)sulfonyl]piperazin-2-one

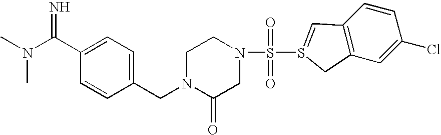

Part 1. To a solution of phenylmethyl 4-[(4-cyanophenyl)methyl]-3-oxopiperazinecarboxylate (1.29. g, 6 mmol) in pyridine (15 mL) at rt, was added 6-chlorobenzo[b]thiophene-2-sulfonyl chloride (1.33 g, 5 mmol). The mixture was stirred at rt overnignt and evaporated in vacuo. The residue was dissolved in EtOAc and H₂O. The organic layer was washed with water, 1N HCl, sat. NaHCO₃, sat. NaCl, dried over Na₂SO₄ and evaporated to give 4-({4-[(6-chlorobenzo[b]thiophen-2-yloxy)sulfinyl]-2-oxopiperazinyl}methyl)benzenecarbonitrile as a solid (1.54 g, 69% yield). MS found (M+H)+: 446.1.

Part 2. A stream of H₂S (g) was bubbled through a solution of 4-({4-[(6-chlorobenzo[b]thiophen-2-yloxy)sulfinyl]-2-oxopiperazinyl}methyl)benzenecarbonitrile (438 mg, 0.985 mmol) in 9 mL pyridine and 1 mL NEt₃ until saturation. The mixture was stirred at rt for 24 hr and evaporated. The resulting residue was treated with MeI (0.613 mL, 9.85 mmol) in 5 mL acetone at reflux temperature for 1 hr and concentrated to dryness. The resulting residue was treated with a mixture of dimethylamine (2.48 mL of 2 M solution in THE, 4.92 mmol) in acetic acid (0.42 mL, 7.38 mmol) and 16 mL methanol at it for 30 min. The solvent was removed at reduced pressure and the crude product was purified by HPLC (C18 reversed phase) eluting with 0.1% TFA in H₂O/CH₃CN to give 1-({4-[(Dimethylamino)iminomethyl]phenyl}methyl)-4-[(6-chlorobenzo[b]thiophen-2-yl)sulfonyl]piperazin-2-one. MS found: (M+H)⁺: 491.1.

### Example 17

### 4-[(6-Chlorobenzo[b]thiophen-2-yl)sulfonyl]-1-{[4-(1-methyl(2-imidazolin-2-yl))phenyl]methyl}piperazin-2-one

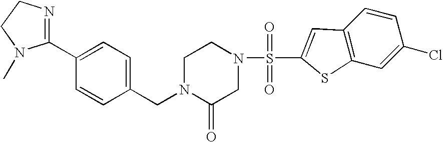

The titled compound was synthesized using a similar procedure to that described in Example 16 using N-methylethylenediamine instead of dimethylamine in Part 2. MS found: (M+H)+: 503.1.

### Example 18

### 1-{[4-(Azetidinyliminomethyl)phenyl]methyl}-4-[(6-chlorobenzo[b]thiophen-2-yl)sulfonyl]piperazin-2-one

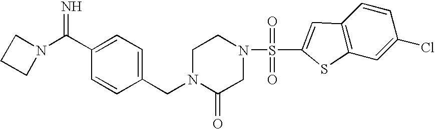

The titled compound was synthesized using a similar procedure to that described in Example 16 using azetidine instead of dimethylamine in Part 2. MS found: (M+H)+: 503.1.

### Example 19

### 4-[(6-Chlorobenzo[b]thiophen-2-yl)sulfonyl]-1-{[4-(iminopyrrolidinylmethyl)phenyl]methyl}piperazin-2-one

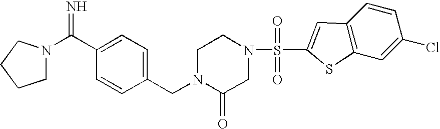

The titled compound was synthesized using a similar procedure to that described in Example 16 using pyrrolidine instead of dimethylamine in Part 2. MS found: (M+H)+: 517.1.

### Example 20

### Ethyl 1-{[4-({4-[(6-chlorobenzo[b]thiophen-2-yl)sulfonyl]-2-oxopiperazinyl}methyl)phenyl]iminomethyl}piperidine-4-carboxylate

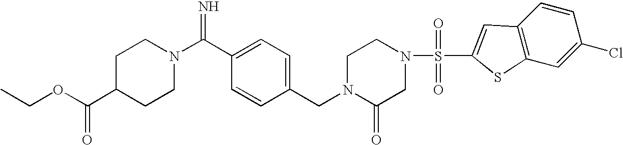

The titled compound was synthesized using a similar procedure to that described in Example 16 using ethyl isonipecotate instead of dimethylamine in Part 2. MS found: (M+H)+: 603.1.

### Example 21

### 1-{[4-({4-[(6-Chlorobenzo[b]thiophen-2-yl)sulfonyl]-2-oxopiperazinyl}methyl)phenyl]iminomethyl}piperidine-4-carboxylic acid

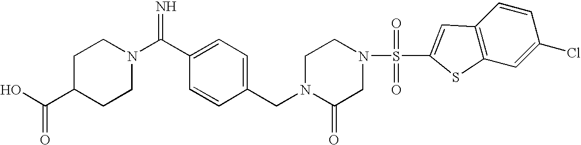

A solution of Ethyl 1-{[4-({4-[(6-chlorobenzo[b]thiophen-2-yl)sulfonyl]-2-oxopiperazinyl}methyl)phenyl]iminomethyl}piperidine-4-carboxylate. (46 mg, 0.077 mmol) in 2 mL of methanol and 2 mL of H₂O was treated with LiOH (6.46 mg, 0.154 mmol) at rt for 2 h. Methanol was evaporated, and the H₂O layer was acidified with 1N HCl until pH˜1-2. The H₂O layer was extracted with EtOAc. The organic layer was dried over MgSO₄, filtered and evaporated in vacuo and the crude product was purified by HPLC (C18 reversed phase) eluting with 0.1% TFA in H₂O/CH₃CN to give 1-{[4-({4-[(6-Chlorobenzo[b]thiophen-2-yl)sulfonyl]-2-oxopiperazinyl}methyl)phenyl]iminomethyl}piperidine-4-carboxylic acid. MS found: (M+H)⁺: 575.1.

### Example 22

### 1-{[4-(Azetidinyliminomethyl)phenyl]methyl}-4-[(5-chloroindol-2-yl)sulfonyl]piperazin-2-one

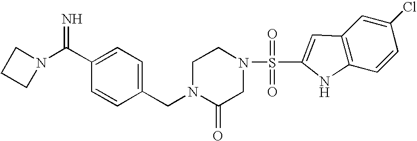

Part 1: To a 25 mL round bottom flask was added phenylmethyl 3-oxopiperazinecarboxylate (1.17 g, 5 mmol), dry DMF (10 mL), -bromo-p-tolunitrile (1 g, 5 mmol) and Cs₂CO₃ (4.89 g, 15 mmol). The mixture was stirred at room temperature for 20 hours. EtOAc and water were added to the reaction. The organic layer was washed with water, dried over MgSO4, and concentrated in vacuo to afford phenylmethyl 4-[(4-cyanophenyl)methyl]-3-oxopiperazinecarboxylate as light yellow organic oil (1.43 g, 82%). MS found: (M+H)⁺: 350.

Part 2. A mixture of phenylmethyl 4-[(4-cyanophenyl)methyl]-3-oxopiperazinecarboxylate (1 g, 2.87 mmol) and 10% Pd/C (160 mg) in MeOH (10 mL) was stirred under balloon H₂ (1 atm) for 3 hrs. The Pd/C was filtered off through a celite bed. The filtrate was evaporated to give 4-[(2-oxopiperazinyl)methyl]benzenecarbonitrile (600 mg, 97%). MS found: (M+H)+: 216.1.

Part 3: A solution of 5-chloroindole (25.7 g, 169 mmol) in anhydrous THF (500 mL) was cooled with a dry ice-acetone bath, and n-BuLi (80 mL of 2.45 M solution in hexanes) was added dropwise over 15 minutes. The reaction was stirred in the cold for 10 minutes, then a solution of di-t-butyl dicarbonate (46.2 g, 212 mmol) in THF (150 mL) was added dropwise over 20 minutes. The reaction was stirred at room temperature overnight. EtOAc (500 mL) and 50 mL of water (50 mL) were added. The aqueous layer was extracted with EtOAc (300 mL), and the combined organics were washed with water (200), dried over MgSO4, filtered and the Filtrate were concentrated to give brown oil. This oil was subjected to flash column chromatography on silica gel first with 100% hexane, then with 0.5% EtOAc/Hexanes then with 1% EtOAc/Hexanes as eluents. The appropriate factions were combined and concentrated to give tert-butyl 5-chloroindolecarboxylate (39.34 g, 92%) as clear, nearly colorless oil that crystallized on the high vacuum pump overnight.

Part 4: A solution of tert-butyl 5-chloroindolecarboxylate (20.0 g, 79.5 mmol) from Part 1 in THF (200 mL) was cooled with a dry ice-acetone bath under Ar, and n-BuLi (44 mL of 2.0 M solution in hexanes, 87.5 mmol) was added over 15 minutes. The reaction mixture was allowed to stir in the cold for 10 minutes, then added dropwise (over 10 minutes) via double-ended needle to a pre-cooled (with a dry ice-acetone bath) solution of SO₂ (80 g) in THF (100 mL). The reaction was allowed to stir at room temperature for 2 hrs. then concentrated to give a brown foam. This foam was dissolved in methylene chloride (200 mL) and the solution was cooled to OC in an ice-water bath. To this was added sulfuryl chloride (7 mL, 87.5 mmol) dropwise over 5 minutes. The ice bath was removed and the reaction was stirred at room temperature overnight. Much solid (LiCl) had begun to form during the addition of SO₂Cl₂. The reaction was filtered and the filtrate was concentrated to give a dark residue. This residue was washed through a plug of silica gel with 1L of 30% DCM/Hexanes. The solvent was evaporated and the residue was subjected to flash column chromatography on silica gel using first 10% DCM/Hexanes, then 15% DCM/Hexanes. The appropriate fractions were combined and concentrated to give tert-butyl 5-chloro-2-(chlorosulfonyl)indolecarboxylate (14.34 g, 52%) as an off-white solid.

Part 5: Phenylmethyl 4-[(4-cyanophenyl)methyl]-3-oxopiperazinecarboxylate from Part 2 (0.35 g, 1 mmol) was dissolved in methylene chloride (5 mL) and pyridine (5 mL). Five minutes later 0.3502 g of tert-butyl 5-chloro-2-(chlorosulfonyl)indolecarboxylate (0.35 g) from Part 4 was added to the reaction. The mixture was stirred at room temperature for five hours. The solvent was removed in vacuo. Water and methylene chloride was added to the crude brown oil. The organic layers were combined, dried over MgSO₄, concentrated in vacuo to tert-butyl 5-chloro-2-({4-[(4-cyanophenyl)methyl]-3-oxopiperazinyl}sulfinyloxy)indolecarboxylate as a light yellow solid (0.40 g, 75%). MS found: (M+H)⁺: 530.

Part 6: tert-Butyl 5-chloro-2-({4-[(4-cyanophenyl)methyl]-3-oxopiperazinyl}sulfinyloxy)indolecarboxylate from Part 5 (50 mg, 0.09 mmol) was dissolved in dry EtOH (10 mL) and HCl gas was bubbled through the solution until saturation. The mixture was stirred at room temperature for 24 hours. The solvent was removed in vacuo and the residue was redissolved in EtOH, to this was added azetidine HCl salt (50 mg, 0.53 mmol) and triethyl amine (84 mM, 0.6 mmol). The mixture was stirred at room temperature for 5 hours and purified by HPLC (C 18 reversed phase) eluting with 0.1% TFA in H₂O/CH₃CN to afford 1-{[4-(Azetidinyliminomethyl)phenyl]methyl}-4-[(5-chloroindol-2-yl)sulfonyl]piperazin-2-one (34 mg, 74%). MS found (M+H)⁺: 486.

### Example 23

### Ethyl 1-{[4-({4-[(5-chloroindol-2-yl)sulfonyl]-2-oxopiperazinyl}methyl)phenyl]iminomethyl}piperidine-4-carboxylate

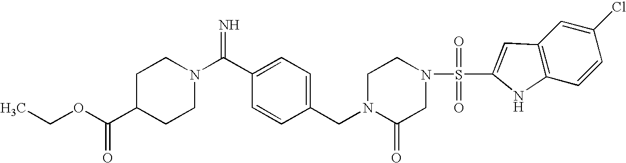

The titled compound was synthesized using a similar procedure to that described in Example 22 using ethyl isonipecotate instead of azetidine in Part 6. MS found: (M+H)⁺: 586.

### Example 24

### 1-({4-[(8-Aza-1,4-dioxaspiro[4.5]dec-8-yl)iminomethyl]phenyl}methyl)-4-[(5-chloroindol-2-yl)sulfonyl]piperazin-2-one

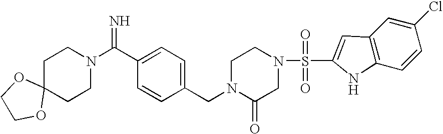

The titled compound was synthesized using a similar procedure to that described in Example 22 using 1,4-dioxa-8-azaspiro[4,4]decane instead of azetidine in Part 6. MS found: (M+H)⁺: 572.

### Example 25

### 4-[(5-chloroindol-2-yl)sulfonyl]-1-({4-[(ethylmethylamino)iminomethyl]phenyl}methyl)piperazin-2-one

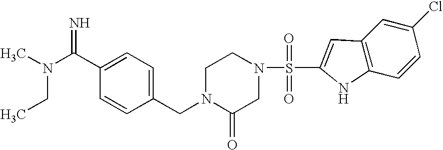

The titled compound was synthesized using a similar procedure to that described in Example 22 using N-ethylmethylamine instead of azetidine in Part 6. MS found: (M+H)⁺: 488.

### Example 26

### 1-[(4-{[4-(Dimethylamino)piperidyl]iminomethyl}phenyl)methyl]4-[(6-chloroindol-2-yl)sulfonyl]piperazin-2-one

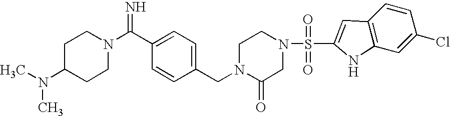

The titled compound was synthesized using a similar procedure to that described in Example 22 using 4-(dimethylamino)-piperidine instead of azetidine in Part 6. MS found: (M+H)⁺: 557.

### Example 27

### 1-({4-[(Dimethylamino)iminomethyl]phenyl}methyl)-4-[(6-chloroindol-2-yl)sulfonyl]piperazin-2-one

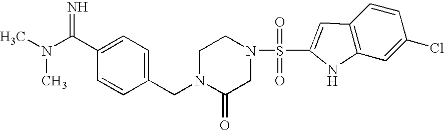

The titled compound was synthesized using a similar procedure to that described in Example 22 using dimethylamine instead of azetidine in part 6. MS found: (M+H)⁺: 474.

### Example 28

### 4-[(6-Chloroindol-2-yl)sulfonyl]-1-{[4-(iminopiperidylmethyl)phenyl]methyl}piperazin-2-one

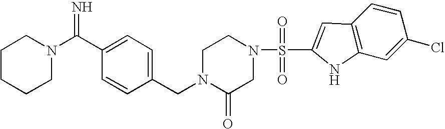

The titled compound was synthesized using a similar procedure to that described in Example 22 piperidine instead of azetidine in part 6. MS found: (M+H)⁺: 514.

### Example 29

### 4-[(6-Chloroindol-2-yl)sulfonyl]-1-{[4-(iminopyrrolidinylmethyl)phenyl]methyl}piperazin-2-one

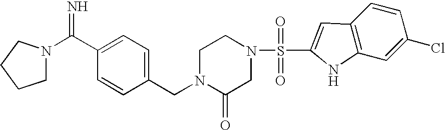

The titled compound was synthesized using a similar procedure to that described in Example 22 pyrrolidine instead of azetidine in part 6. MS found: (M+H)⁺: 500.

### Example 30

### 1-{[4-({[2-(Dimethylamino)ethyl]methylamino}iminomethyl)phenyl]methyl}-4-[(6-chloroindol-2-yl)sulfonyl]piperazin-2-one

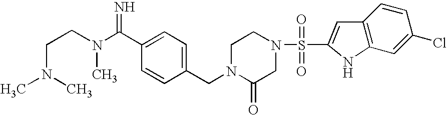

The titled compound was synthesized using a similar procedure to that described in Example 22 using N, N, N′-trimethylethylenediamine instead of azetidine in Part 6. MS found: (M+H)⁺: 531.

### Example 31

### 4-[(6-Chlorobenzo[b]thiophen-2-yl)sulfonyl]-1-{[4-(1-methyl(2-imidazolin-2-yl))phenyl]methyl}piperazin-2-one

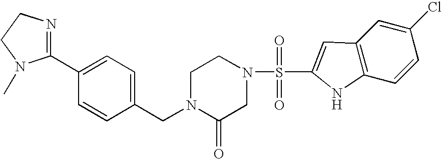

The titled compound was synthesized using a similar procedure to that described in Example 22 using N-methylethylenediamine instead of azetidine in part 6. MS found: (M+H)⁺: 486.

### Example 32

### 4-[(5-Chloroindol-2-yl)sulfonyl]-1-({4-[imino(methylpropylamino)methyl]phenyl}methyl)piperazin-2-one

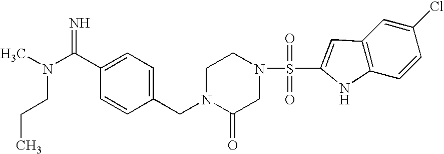

The titled compound was synthesized using a similar procedure to that described in Example 22 using N-methyl-N-propylamine instead of azetidine in Part 6. MS found: (M+H)⁺: 502.

### Example 33

### 1-({4-[(4-Bromopiperidyl)iminomethyl]phenyl}methyl)-4-[(5-chloroindol-2-yl)sulfonyl]piperazin-2-one

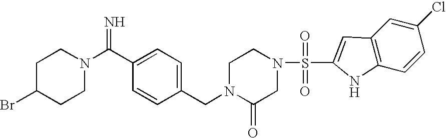

The titled compound was synthesized using a similar procedure to that described in Example 22 using 4-bromopiperidine instead of azetidine in Part 6. MS found: (M+H)⁺: 592.

### Example 34

### 4-[(5-chloroindol-2-yl)sulfonyl]-1-({4-[imino(methylprop-2-enylamino)methyl]phenyl}methyl)piperazin-2-one

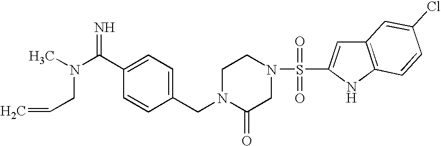

The titled compound was synthesized using a similar procedure to that described in Example 22 using N-methylallylamine instead of azetidine in Part 6. MS found: (M+H)⁺: 500.

### Example 35

### 4-[(5-Chloroindol-2-yl)sulfonyl]-1-[(4-{[(2-furylmethyl)methylamino]iminomethyl}phenyl)methyl]piperazin-2-one

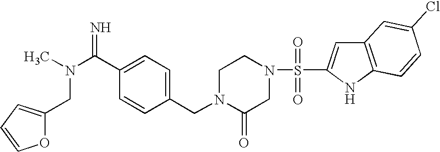

The titled compound was synthesized using a similar procedure to that described in Example 22 using N-methylfurfurylamine instead of azetidine in Part 6. MS found: (M+H)⁺: 540.

### Example 36

### 1-({4-[(Butylmethylamino)iminomethyl]phenyl}methyl)-4-[(5-chloroindol-2-yl)sulfonyl]piperazin-2-one

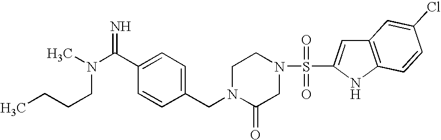

The titled compound was synthesized using a similar procedure to that described in Example 22 using N-methylbutylamine instead of azetidine in Part 6. MS found: (M+H)⁺: 516.

### Example 37

### 4-[(5-Chloroindol-2-yl)sulfonyl]-1-({4-[imino(4-oxopiperidyl)methyl]phenyl}methyl)piperazin-2-one

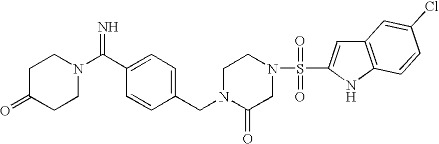

1-({4-[(8-Aza-1,4-dioxaspiro[4.5]dec-8-yl)iminomethyl]phenyl}methyl)-4-[(5-chloroindol-2-yl)sulfonyl]piperazin-2-one from example 24 (10 mg) was dissolved in a solution of 4N HCl in dioxane (3 mL). The mixture was refluxed for 6 hours and concentrated in vacuo. The residue was purified by HPLC (C18 reversed phase) eluting with 0.1% TFA in H₂O/CH₃CN to HPLC to afford 4-[(5-Chloroindol-2-yl)sulfonyl]-1-({4-[imino(4-oxopiperidyl)methyl]phenyl}methyl)piperazin-2-one.(7 mg, 71%). MS found: (M+H)⁺: 528.

### Example 38

### 4-[(5-Chloroindol-2-yl)sulfonyl]-1-({4-[(cyclohexylmethylamino)iminomethyl]phenyl}methyl)piperazin-2-one

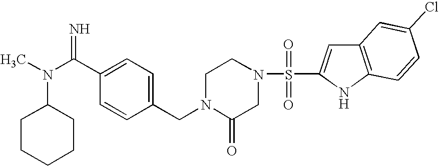

The titled compound was synthesized using a similar procedure to that described in Example 22 using N-methylcyclohexylamine instead of azetidine in Part 6. MS found: (M+H)⁺: 542.

### Example 39

### 1-{[4-({4-[(5-Chloroindol-2-yl)sulfonyl]-2-oxopiperazinyl}methyl)phenyl]iminomethyl}piperidine-4-carboxylic acid

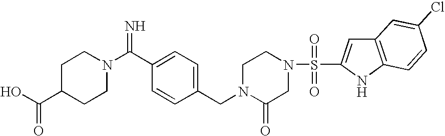

The titled compound was synthesized using a similar procedure to that described in Example 3 using Ethyl 1-{[4-({4-[(5-chloroindol-2-yl)sulfonyl]-2-oxopiperazinyl}methyl)phenyl]iminomethyl}piperidine-4-carboxylate from example 23. MS found: (M+H)⁺: 558.

### Example 40

### 4-[(5-Chloroindol-2-yl)sulfonyl]-1-[(4-{imino[methyl(2-phenylethyl)amino]methyl}phenyl)methyl]piperazin-2-one

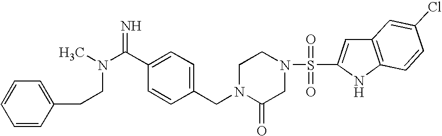

The titled compound was synthesized using a similar procedure to that described in Example 22 using N-methylphenethylamine instead of azetidine in Part 6. MS found: (M+H)⁺: 564.

### Example 41

### 4-[(5-Chloroindol-2-yl)sulfonyl]-1-[(4-{imino[methyl(2-(2-pyridyl)ethyl)amino]methyl}phenyl)methyl]piperazin-2-one

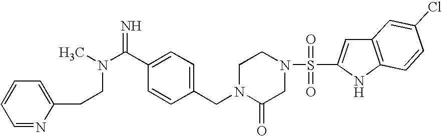

The titled compound was synthesized using a similar procedure to that described in Example 22 using 4-[2-(methylamino)ethyl]pyridine instead of azetidine in Part 6. MS found: (M+H)⁺: 565.

### Example 42

### 1-({4-[(But-3-ynylmethylamino)iminomethyl]phenyl}methyl)-4-[(5-chloroindol-2-yl)sulfonyl]piperazin-2-one

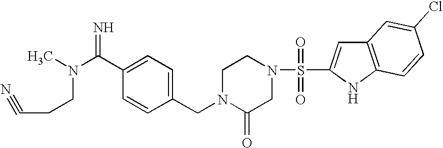

The titled compound was synthesized using a similar procedure to that described in Example 22 using N-methyl- -alaninenitrile instead of azetidine in Part 6. MS found: (M+H)⁺: 513.

### Example 43

### 4-[(5-Chloroindol-2-yl)sulfonyl]-1-[(4-{imino[methylbenzylamino]methyl}phenyl)methyl]piperazin-2-one

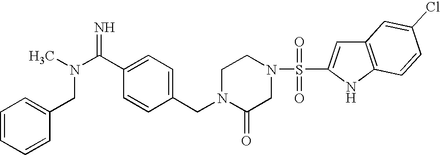

The titled compound was synthesized using a similar procedure to that described in Example 22 using N-methylbenzylamine instead of azetidine in Part 6. MS found: (M+H)⁺: 550.

### Example 44

### 1-[(4-{[(2,2-Dimethoxyethyl)methylamino]iminomethyl}phenyl)methyl]-4-[(5-chloroindol-2-yl)sulfonyl]piperazin-2-one

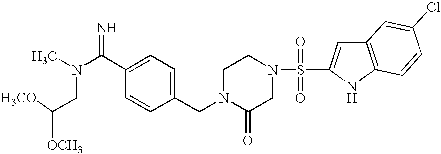

The titled compound was synthesized using a similar procedure to that described in Example 22 using methylaminoacetaldehyde dimethylacetal instead of azetidine in Part 6. MS found: (M+H)⁺: 548.

### Example 45

### 4-[(5-Chloroindol-2-yl)sulfonyl]-1-({4-[imino(4-pyrrolidinylpiperidyl)methyl]phenyl}methyl)piperazin-2-one

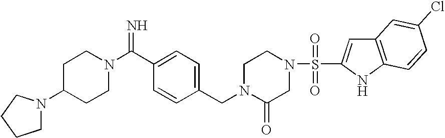

The titled compound was synthesized using a similar procedure to that described in Example 22 using 4-pyrrolidinopiperidine instead of azetidine in Part 6. MS found: (M+H)⁺: 583.

### Example 46

### 4-[(5-Chloroindol-2-yl)sulfonyl]-1-[(4-{[(1,3-dioxolan-2-ylmethyl)methylamino]iminomethyl}phenyl)methyl]piperazin-2-one

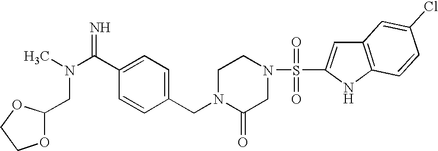

The titled compound was synthesized using a similar procedure to that described in Example 22 using 2-methylaminomethyl-1,3-dioxalone instead of azetidine in Part 6. MS found: (M+H)⁺: 546.

### Example 47

### 4-[(5-Chloroindol-2-yl)sulfonyl]-1-({4-[imino(methylamino)methyl]phenyl}methyl)piperazin-2-one

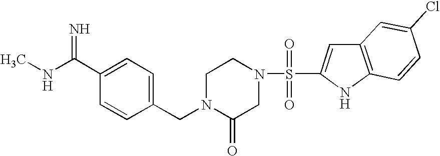

The titled compound was synthesized using a similar procedure to that described in Example 22 using methylamine instead of azetidine in Part 6. MS found: (M+H)⁺: 460.

### Example 48

### 4-[(5-Chloroindol-2-yl)sulfonyl]-1-[(4-{imino[methyl(2-oxoethyl)amino]methyl}phenyl)methyl]piperazin-2-one

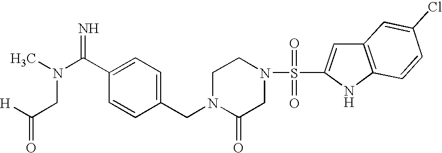

The titled compound was synthesized using a similar procedure to that described in Example 37 using 4-[(5-Chloroindol-2-yl)sulfonyl]-1-[(4-{[(1,3-dioxolan-2-ylmethyl)methylamino]iminomethyl}phenyl)methyl]piperazin-2-one from example 46. MS found: (M+H)⁺: 502.

### Example 49

### 4-[(5-Chloroindol-2-yl)sulfonyl]-1-({4-[(ethynylmethylamino)iminomethyl]phenyl}methyl)piperazin-2-one

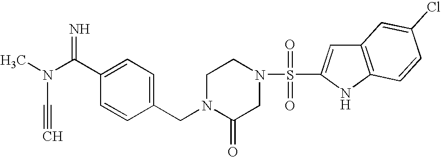

The titled compound was synthesized using a similar procedure to that described in Example 37 as a minor product. MS found: (M+H)⁺: 484.

### Example 50

### 4-[(5-Chloroindol-2-yl)sulfonyl]-1-({4-[imino(methylprop-2-ynylamino)methyl]phenyl}methyl)piperazin-2-one

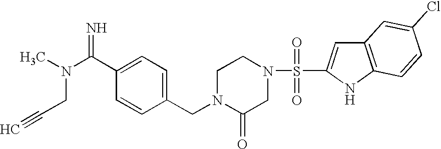

The titled compound was synthesized using a similar procedure to that described in Example 22 using N-methylpropargylamine instead of azetidine in Part 6. MS found: (M+H)⁺: 498.

### Example 51

### 1-{[4-(Azetidinylazetidinylidenemethyl)phenyl]methyl}-4-[(5-chloroindol-2-yl)sulfonyl]piperazin-2-one

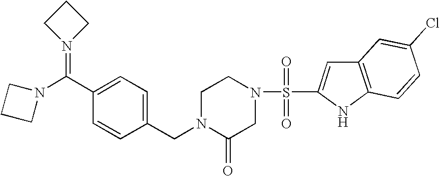

The titled compound was synthesized using a similar procedure to that described in Example 22 as a minor product. MS found: (M+H)⁺: 529.

### Example 52

### 4-[(6-Chlorobenzo[b]thiophen-2-yl)sulfonyl]-1-({4-[imino(methylprop-2-ynylamino)methyl]phenyl}methyl)piperazin-2-one

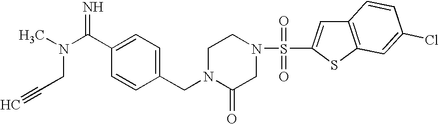

The titled compound was synthesized using a similar procedure to that described in Example 22 using 5-chloro-2-thienyloxyacetic acid (Ewing, W. R., WO 0032590, 2000) instead of tert-butyl 5-chloro-2-(chlorosulfonyl)indolecarboxylate in Part 5 and N-methylpropargylamine instead of azetidine in Part 6. MS found: (M+H)⁺: 515.

### Example 53

### 4-[(6-Chlorobenzo[b]thiophen-2-yl)sulfonyl]-1-[(4-{imino[methyl(2-phenylethyl)amino]methyl}phenyl)methyl]piperazin-2-one

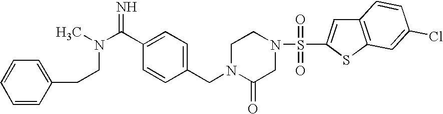

The titled compound was synthesized using a similar procedure to that described in Example 22 using 5-chloro-2-thienyloxyacetic acid instead of tert-butyl 5-chloro-2-(chlorsulfonyl)indolecarboxylate in Part 5 and N-methylphenethylamine instead of azetidine in Part 6. MS found: (M+H)⁺: 581.

### Example 54

### 4-[(6-Chlorobenzo[b]thiophen-2-yl)sulfonyl]-1-[(4-{imino [methyl(2-(2-pyridyl)ethyl)amino]methyl}phenyl)methyl]piperazin-2-one

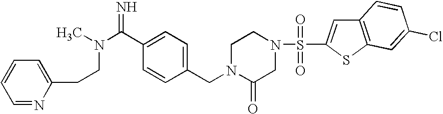

The titled compound was synthesized using a similar procedure to that described in Example 22 using 5-chloro-2-thienyloxyacetic acid instead of tert-butyl 5-chloro-2-(chlorosulfonyl)indolecarboxylate in Part 5 and 4-[2-(methylamino)ethyl]pyridine instead of azetidine in Part 6. MS found: (M+H)⁺: 582.

### Example 55

### 4-[(6-Chlorobenzo[b]thiophen-2-yl)sulfonyl]-1-({4-[imino(methylprop-2-enylamino)methyl]phenyl}methyl)piperazin-2-one

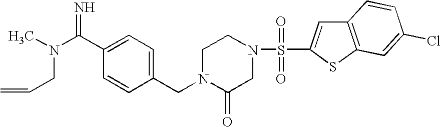

The titled compound was synthesized using a similar procedure to that described in Example 22 using 5-chloro-2-thienyloxyacetic acid instead of tert-butyl 5-chloro-2-(chlorosulfonyl)indolecarboxylate in Part 5 and N-methylallylamine instead of azetidine in Part 6. MS found: (M+H)⁺: 517.

### Example 56

### 4-[(6-chlorobenzo[b]thiophen-2-yl)sulfonyl]-1-[(4-{[(1,3-dioxolan-2-ylmethyl)methylamino]iminomethyl}phenyl)methyl]piperazin-2-one

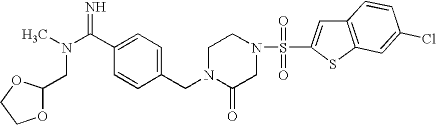

The titled compound was synthesized using a similar procedure to that described in Example 22 using 5-chloro-2-thienyloxyacetic acid instead of tert-butyl 5-chloro-2-(chlorosulfonyl)indolecarboxylate in Part 5 and 2-methylaminomethyl-1,3-dioxalone instead of azetidine in Part 6. MS found: (M+H)⁺: 563.1.

### Example 57

### Methyl (2R)-4-[(6-chlorobenzo[b]thiophen-2-yl)sulfonyl]-1-[(4-{[1,3-dioxolan-2-ylmethyl)methylamino]iminomethyl}phenyl)methyl]-6-oxopiperazine-2-carboxylate

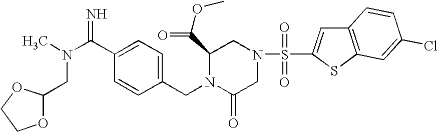

Part 1: To a precooled (ice bath) mixture of Z-D-Dap-OH (7.1 g, 29.8 mmol) and anhydrous MeOH (23 mL), was added slowly SOCl₂, (2.2 mL, 30 mmol). The mixture was allowed to warm to room temperature and stirred at this temperature for 18 hours. The solvent was removed in vacuo. The product was crystallized to afford 3-amino-N-[(benzyloxy)carbonyl]-D-alanine methyl ester hydrochloride (7.5 g, 87%).

Part 2: To a stirred mixture of 3-amino-N-[(benzyloxy)carbonyl]-D-alanine methyl ester from Part 1 (free base: 6.2 g, 24.5 mmol), DIEA (3.5g, 26.7mmol), and anhydrous THF (65 mL), was added methyl bromoacetate (3.7 g, 24.5 mmol) in anhydrous THF (10 ml). The mixture was stirred at rt for 24 hours. The suspension was filtered and the filtrate evaporated. The residue was treated with Et₂O, filtered, and the solvent evaporated. The oily residue (7.8 g) was dissolved in EtOH (100 mL) and hydrogenated over 10% Pd/C. The catalyst was filtered off, the solvent removed and the product crystallized form EtOH/Et2O to afford 2.7 g (70%) of methyl (2R)-6oxopiperazine-2-carboxylate as colorless crystals.

Part 3: The titled compound was synthesized using a similar procedure to that described in Example 1 using methyl (2R)-6-oxopiperazine-2-carboxylate instead of ethyl 3-oxopiperazine-2-acetate in Part 1 and 2-methylaminomethyl-1,3-dioxalone instead of dimethylamine in Part 3. MS found: (M+H)⁺: 621.

### Example 58

### (2R)-4-[(6-Chlorobenzo[b]thiophen-2-yl)sulfonyl]-1-[(4-{[(1,3-dioxolan-2-ylmethyl)methylamino]iminomethyl}phenyl)methyl]-6-oxopiperazine-2-carboxylic acid

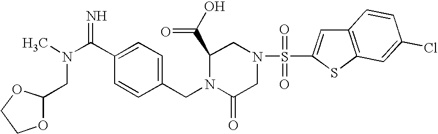

The titled compound was synthesized using a similar procedure to that described in Example 3 using Methyl (2R)-4-[(6-chlorobenzo[b]thiophen-2-yl)sulfonyl]-1-[(4-{[(1,3-dioxolan-2-ylmethyl)methylamino]iminomethyl}phenyl)methyl]-6-oxopiperazine-2-carboxylate. MS found: (M+H)⁺: 607.1.

### Example 59

### Methyl (2R)-1-{[4-(azetidinyliminomethyl)phenyl]methyl}-4-[(6-chlorobenzo[b]thiophen-2-yl)sulfonyl]-6-oxopiperazine-2-carboxylate

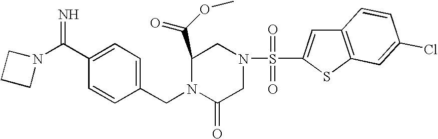

The titled compound was synthesized using a similar procedure to that described in Example 1 using methyl (2R)-6-oxopiperazine-2-carboxylate instead of ethyl 3-oxopiperazine-2-acetate in Part 1 and azetidine instead of dimethylamine in Part 3. MS found: (M+H)⁺: 561.0.

### Example 60

### (2R)-1-{[4-(Azetidinyliminomethyl)phenyl]methyl}-4-[(6-chlorobenzo[b]thiophen2-yl)sulfonyl]-6-oxopiperazine-2-carboxylic acid

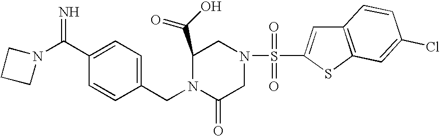

The titled compound was synthesized using a similar procedure to that described in Example 3. MS found: (M+H)⁺: 547.1.

### Example 61

### Methyl (2R)-4-[(6-chlorobenzo[b]thiophen-2-yl)sulfonyl]-1-{[4-(iminopyrrolidinylmethyl)phenyl]methyl}-6-oxopiperazine-2-carboxylate

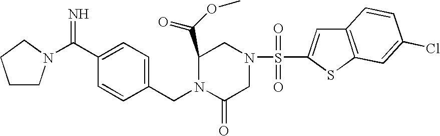

The titled compound was synthesized using a similar procedure to that described in Example 1 using methyl (2R)-6-oxopiperazine-2-carboxylate instead of ethyl 3-oxopiperazine-2-acetate in Part 1 and pyrrolidine instead of dimethylamine in Part 3. MS found: (M+H)⁺: 575.1.

### Example 62

### (2R)-4-[(6-Chlorobenzo[b]thiophen-2-yl)sulfonyl]-1-{[4-(iminopyrrolidinylmethyl)phenyl]methyl}-6-oxopiperazine-2-carboxylic acid

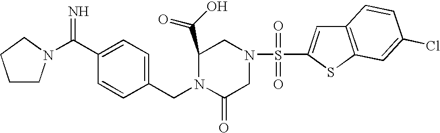

The titled compound was synthesized using a similar procedure to that described in Example 3. MS found: (M+H)⁺: 561.1.

### Example 63

### Methyl (2R)-4-[(6-chlorobenzo[b]thiophen-2-yl)sulfonyl]-1-{[4-(iminopiperidylmethyl)phenyl]methyl}-6-oxopiperazine-2-carboxylate

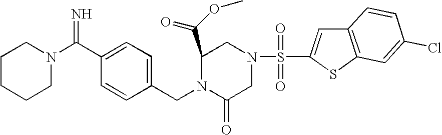

The titled compound was synthesized using a similar procedure to that described in Example 1 using methyl (2R)-6-oxopiperazine-2-carboxylate instead of ethyl 3-oxopiperazine-2-acetate in Part 1 and piperidine instead of dimethylamine in Part 3. MS found: (M+H)⁺: 589.1.

### Example 64

### (2R)-4-[(6-Chlorobenzo[b]thiophen-2-yl)sulfonyl]-1-{[4-(iminopiperidylmethyl)phenyl]methyl}-6-oxopiperazine-2-carboxylic acid

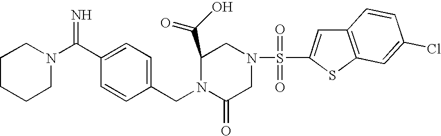

The titled compound was synthesized using a similar procedure to that described in Example 3. MS found: (M+H)⁺: 575.1.

### Example 65

### Methyl (2R)-4-[(6-chlorobenzo[b]thiophen-2-yl)sulfonyl]-1-[(4-{[4-(ethoxycarbonyl)piperidyl]iminomethyl}phenyl)methyl]-6-oxopiperazine-2-carboxylate

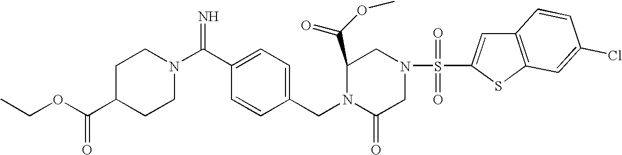

The titled compound was synthesized using a similar procedure to that described in Example 1 using methyl (2R)-6-oxopiperazine-2-carboxylate instead of ethyl 3-oxopiperazine-2-acetate in Part 1 and ethyl isonipecotate instead of dimethylamine in Part 3. MS found: (M+H)⁺: 661.1.

### Example 66

### (2R)-1-({4-[(4-Carboxypiperidyl)iminomethyl]phenyl}methyl)-4-[(6-chlorobenzo[b]thiophen-2-yl)sulfonyl]-6-oxopiperazine-2-carboxylic acid

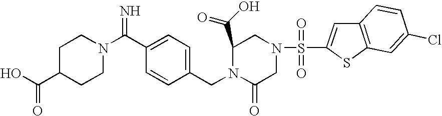

The titled compound was synthesized using a similar procedure to that described in Example 3. MS found: (M+H)⁺: 619.1.

### Example 67

### Methyl (2R)-4-[(6-Chlorobenzo[b]thiophen-2-yl)sulfonyl]-1-{[4-[imino(methylprop-2-enylamino)methyl]phenyl}methyl)-6-oxopiperazine-2-carboxylate

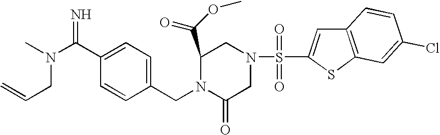

The titled compound was synthesized using a similar procedure to that described in Example 1 using methyl (2R)-6-oxopiperazine-2-carboxylate instead of ethyl 3-oxopiperazine-2-acetate in Part 1 and N-methylallylamine instead of dimethylamine in Part 3. MS found: (M+H)⁺: 575.1.

### Example 68

### (2R)-4-[(6-Chlorobenzo[b]thiophen-2-yl)sulfonyl]-1-({4-[imino(methylprop-2-enylamino)methyl]phenyl}methyl)-6-oxopiperazine-2-carboxylic acid

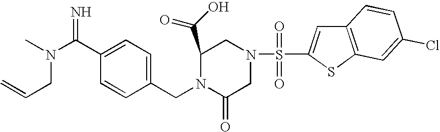

The titled compound was synthesized using a similar procedure to that described in Example 3. MS found: (M+H)⁺: 561.1.

### Example 69

### Methyl (2R)-4-[(6-chlorobenzo[b]thiophen-2-yl)sulfonyl]-1-({4-[imino(methylprop-2-ynylamino)methyl]phenyl}methyl)-6-oxopiperazine-2-carboxylate

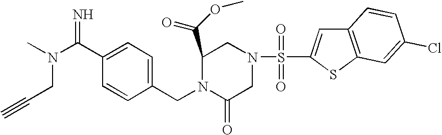

The titled compound was synthesized using a similar procedure to that described in Example 1 using methyl (2R)-6-oxopiperazine-2-carboxylate instead of ethyl 3-oxopiperazine-2-acetate in Part 1 and N-methylpropargylamine instead of dimethylamine in Part 3. MS found: (M+H)⁺: 573.1.

### Example 70

### (2R)-4-[(6-Chlorobenzo[b]thiophen-2-yl)sulfonyl]-1-({4-[imino(methylprop-2-ynylamino)methyl]phenyl}methyl)-6-oxopiperazine-2-carboxylic acid

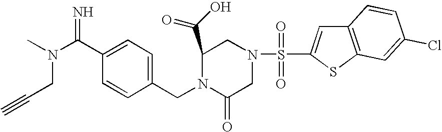

The titled compound was synthesized using a similar procedure to that described in Example 3. MS found: (M+H)⁺: 559.1.

### Example 71

### Methyl (2R)4-[(6-chlorobenzo[b]thiophen-2-yl)sulfonyl]-1-({4-[imino(methylamino)methyl]phenyl}methyl)-6-oxopiperazine-2-carboxylate

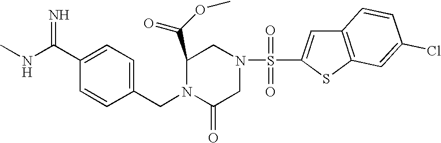

The titled compound was synthesized using a similar procedure to that described in Example 1 using methyl (2R)-6-oxopiperazine-2-carboxylate instead of ethyl 3-oxopiperazine-2-acetate in Part 1 and N-methylamine instead of dimethylamine in Part 3. MS found: (M+H)⁺: 535.1.

### Example 72

### (2R)-4-[(6-Chlorobenzo[b]thiophen-2-yl)sulfonyl]-1-({4-[imino(methylamino)methyl]phenyl}methyl)-6-oxopiperazine-2-carboxylic acid

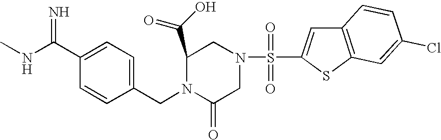

The titled compound was synthesized using a similar procedure to that described in Example 3. MS found: (M+H)⁺: 521.1.

### Example 73

### Methyl (2R)-4-[(6-chlorobenzo[b]thiophen-2-yl)sulfonyl]-1-[(4-{[(2-cyanoethyl)methylamino]iminomethyl}phenyl)methyl]-6-oxopiperazine-2-carboxylate

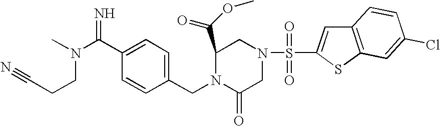

The titled compound was synthesized using a similar procedure to that described in Example 1 using methyl (2R)-6-oxopiperazine-2-carboxylate instead of ethyl 3-oxopiperazine-2-acetate in Part 1 and N-methyl- -alaninenitrile instead of dimethylamine in Part 3. MS found: (+H)⁺: 586.1.

### Example 74

### (2R)-4-[(6-Chlorobenzo[b]thiophen-2-yl)sulfonyl]-1-[(4-{[(2-cyanoethyl)methylamino]iminomethyl}phenyl)methyl]-6-oxopiperazine-2-carboxylic acid

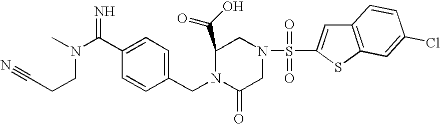

The titled compound was synthesized using a similar procedure to that described in Example 3. MS found: (M+H)⁺: 574.1.

### Example 75

### Methyl (2R)-1-({4-[(dimethylamino)iminomethyl]phenyl}methyl)-4-[(6-chlorobenzo[b]thiophen-2-yl)sulfonyl]-6-oxopiperazine-2-carboxylate

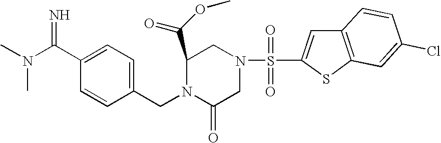

The titled compound was synthesized using a similar procedure to that described in Example 1 using methyl (2R)-6-oxopiperazine-2-carboxylate instead of ethyl 3-oxopiperazine-2-acetate in Part 1. MS found: (M+H)⁺: 549.1.

### Example 76

### (2R)-1-({4-[(Dimethylamino)iminomethyl]phenyl}methyl)-4-[(6-chlorobenzo[b]thiophen-2-yl)sulfonyl]-6-oxopiperazine-2-carboxylic acid

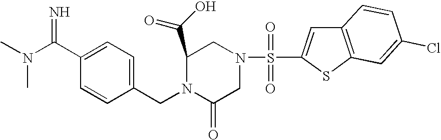

The titled compound was synthesized using a similar procedure to that described in Example 3. MS found: (M+H)⁺: 535.1.

### Example 77

### Methyl (2R)-4-[(6-chlorobenzo[b]thiophen-2-yl)sulfonyl]-1-[(4-{imino[methyl(2-phenylethyl)amino]methyl}phenyl)methyl]-6-oxopiperazine-2-carboxylate

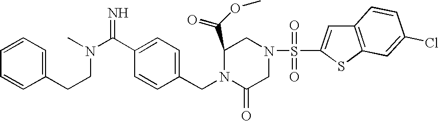

The titled compound was synthesized using a similar procedure to that described in Example 1 using methyl (2R)-6-oxopiperazine-2-carboxylate instead of ethyl 3-oxopiperazine-2-acetate in Part 1 and N-methylphenethylamine instead of dimethylamine in Part 3. MS found: (M+H)⁺: 639.1.

### Example 78

### (2R)-4-[(6-Chlorobenzo[b]thiophen-2-yl)sulfonyl]-1-[(4-{imino[methyl(2-phenylethyl)amino]methyl}phenyl)methyl]-6-oxopiperazine-2-carboxylic acid

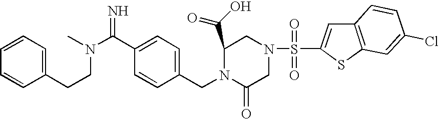

The titled compound was synthesized using a similar procedure to that described in Example 3. MS found: (M+H)⁺: 625.1.

### Example 79

### Methyl (2R)-4-[(6-chlorobenzo[b]thiophen-2-yl)sulfonyl]-1-[(4-{imino [methyl(2-(2-pyridyl)ethyl)amino]methyl}phenyl)methyl]-6-oxopiperazine-2-carboxylate

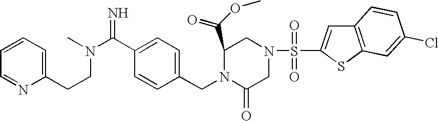

The titled compound was synthesized using a similar procedure to that described in Example 1 using methyl (2R)-6-oxopiperazine-2-carboxylate instead of ethyl 3-oxopiperazine-2-acetate in Part 1 and 4-[2-(methylamino)ethyl]pyridine instead of dimethylamine in Part 3. MS found: (M+H)⁺: 640.1.

### Example 80

### (2R)-4-[(6-Chlorobenzo[b]thiophen-2-yl)sulfonyl]-1-[(4-{imino[methyl(2-(2-pyridyl)ethyl)amino]methyl}phenyl)methyl]-6-oxopiperazine-2-carboxylic acid

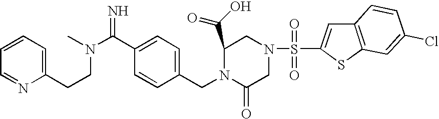

The titled compound was synthesized using a similar procedure to that described in Example 3. MS found: (M+H)⁺: 626.1.

### Example 81

### Methyl (2R)-4-[(6-chlorobenzo[b]thiophen-2-yl)sulfonyl]-1-{[4-(1-methyl(2-imidazolin-2-yl))phenyl]methyl}-6-oxopiperazine-2-carboxylate

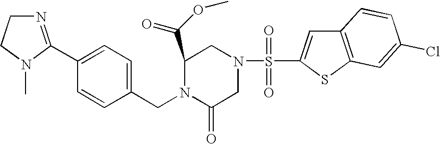

The titled compound was synthesized using a similar procedure to that described in Example 1 using methyl (2R)-6-oxopiperazine-2-carboxylate instead of ethyl 3-oxopiperazine-2-acetate in Part 1 and N-methylethylenediamine instead of dimethylamine in Part 3. MS found: (M+H)⁺: 561.

### Example 82

### (2R)-4-[(6-Chlorobenzo[b]thiophen-2-yl)sulfonyl]-1-{[4-(1-methyl(2-imidazolin-2-yl))phenyl]methyl}-6-oxopiperazine-2-carboxylic acid

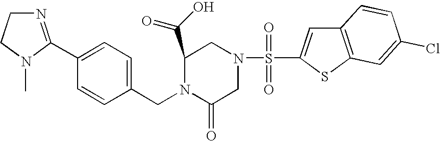

The titled compound was synthesized using a similar procedure to that described in Example 3. MS found: (M+H)⁺: 547.

### Example 83

### Methyl (2R)-1-[(4-{[(1,3-dioxolan-2-ylmethyl)methylamino]iminomethyl}phenyl)methyl]-4-[(5-methylindol-2-yl)sulfonyl]-6-oxopiperazine-2-carboxylate

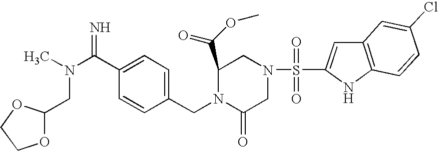

The titled compound was synthesized using a similar procedure to that described in Example 22. MS found: (M+H)⁺: 604.

### Example 84

### (2R)-4-[(5-Chloroindol-2-yl)sulfonyl]-1-[(4-{[(1,3-dioxolan-2-ylmethyl)methylamino]iminomethyl}phenyl)methyl]-6-oxopiperazine-2-carboxylic acid

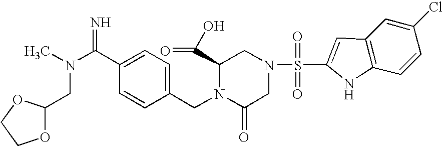

The titled compound was synthesized using a similar procedure to that described in Example 3. MS found: (M+H)⁺: 590.

### Example 85

### Methyl (2R)-1-{[4-(azetidinyliminomethyl)phenyl]methyl}-4-[(5-chloroindol-2-yl)sulfonyl]-6-oxopiperazine-2-carboxylate

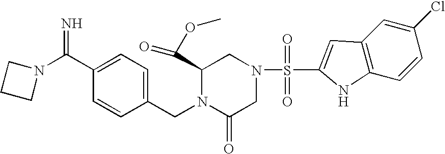

The titled compound was synthesized using a similar procedure to that described in Example 22. MS found: (M+H)⁺: 544.

### Example 86

### (2R)-1-{[4-(Azetidinyliminomethyl)phenyl]methyl}-4-[(5-chloroindol-2-yl)sulfonyl]-6-oxopiperazine-2-carboxylic acid

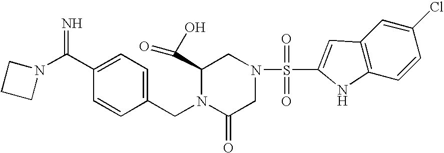

The titled compound was synthesized using a similar procedure to that described in Example 3. MS found: (M+H)⁺: 530.1.

### Example 87

### Methyl (2R)4-1(5-chloroindol-2-yl)sulfonyl]-1-{[4-(iminopyrrolidinylmethyl)phenyl]methyl}-6-oxopiperazine-2-carboxylate

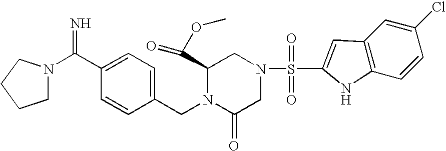

The titled compound was synthesized using a similar procedure to that described in Example 22. MS found: (M+H)⁺: 558.

### Example 88

### (2R)-4-[(5-Chloroindol-2-yl)sulfonyl]-1-[{4-(iminopyrrolidinylmethyl)phenyl]methyl}-6-oxopiperazine-2-carboxylic acid

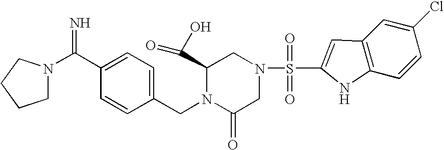

The titled compound was synthesized using a similar procedure to that described in Example 3. MS found: (M+H)⁺: 544.

### Example 89

### Methyl (2R)-4-[(5-chloroindol-2-yl)sulfonyl]-1-{[4-(iminopiperidylmethyl)phenyl]methyl}-6-oxopiperazine-2-carboxylate

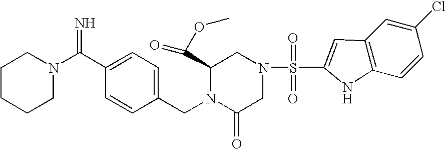

The titled compound was synthesized using a similar procedure to that described in Example 22. MS found: (M+H)⁺: 572.

### Example 90

### (2R)-4-[(5-Chloroindol-2-yl)sulfonyl]-1-{[4-(iminopiperidylmethyl)phenyl]methyl}-6-oxopiperazine-2-carboxylic acid

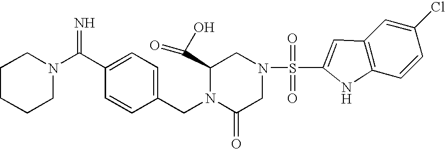

The titled compound was synthesized using a similar procedure to that described in Example 3. MS found: (M+H)⁺: 558.

### Example 91

### Methyl (2R)-4-[(5-chloroindol-2-yl)sulfonyl]-1-[(4-{[4-(ethoxycarbonyl)piperidyl]iminomethyl}phenyl)methyl]-6-oxopiperazine-2-carboxylate

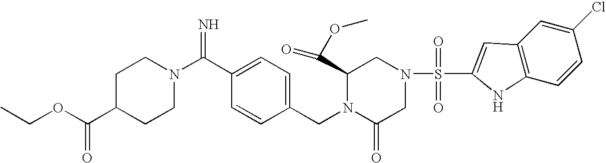

The titled compound was synthesized using a similar procedure to that described in Example 22. MS found: (M+H)⁺: 644.

### Example 92

### (2R)-1-({4-[(4-Carboxypiperidyl)iminomethyl]phenyl}methyl)-4-[(5-chloroindol-2-yl)sulfonyl]-6-oxopiperazine-2-carboxylic acid

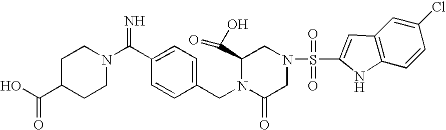

The titled compound was synthesized using a similar procedure to that described in Example 3. MS found: (M+H)⁺: 602.

### Example 93

### Methyl (2R)-4-[(5-chloroindol-2-yl)sulfonyl]-1-({4-[imino(methylprop-2-enylamino)methyl]phenyl}methyl)-6-oxopiperazine-2-carboxylate

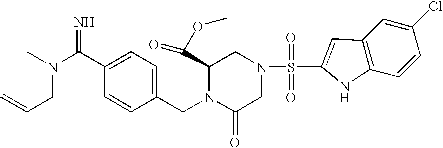

The titled compound was synthesized using a similar procedure to that described in Example 22. MS found: (M+H)⁺: 558.

### Example 94

### (2R)-4-[(5-Chloroindol-2-yl)sulfonyl]-1-({4-[imino(methylprop-2-enylamino)methyl]phenyl}methyl)-6-oxopiperazine-2-carboxylic acid

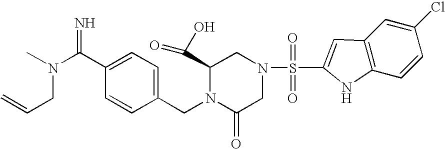

The titled compound was synthesized using a similar procedure to that described in Example 3. MS found: (M+H)⁺: 544.0.

### Example 95

### Methyl (2R)-4-[(5-chloroindol-2-yl)sulfonyl]-1-({4-[imino(methylprop-2-ynylamino)methyl]phenyl}methyl)-6-oxopiperazine-2-carboxylate

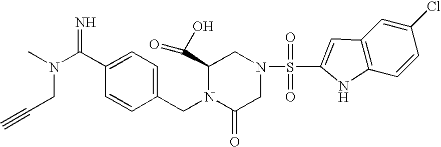

The titled compound was synthesized using a similar procedure to that described in Example 22. MS found: (M+H)⁺: 556.1.

### Example 96

### (2R)-4-[(5-Chloroindol-2-yl)sulfonyl]-1-({4-[imino(methylprop-2-ynylamino)methyl]phenyl}methyl)-6-oxopiperazine-2-carboxylic acid

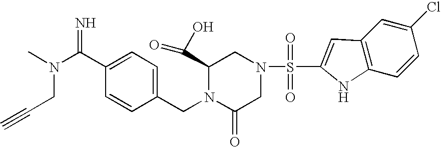

The titled compound was synthesized using a similar procedure to that described in Example 3. MS found: (M+H)⁺: 542.

### Example 97

### Methyl (2R)4-[(5-chloroindol-2-yl)sulfonyl]-1-({4-[imino(methylamino)methyl]phenyl}methyl)-6-oxopiperazine-2-carboxylate

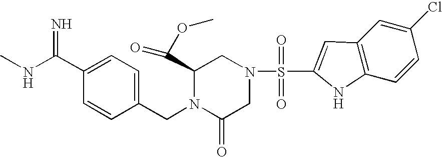

The titled compound was synthesized using a similar procedure to that described in Example 22. MS found: (M+H)⁺: 518.1.

### Example 98

### (2R)-4-[(5-Chloroindol-2-yl)sulfonyl]-1-({4-[imino(methylamino)methyl]phenyl}methyl)-6-oxopiperazine-2-carboxylic acid

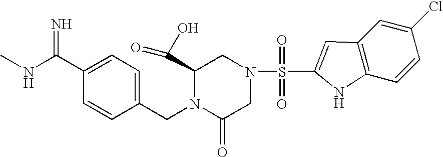

The titled compound was synthesized using a similar procedure to that described in Example 3. MS found: (M+H)⁺: 504.

### Example 99

### Methyl (2R)-4-[(5-chloroindol-2-yl)sulfonyl]-1-[(4-{[(2-cyanoethyl)methylamino]iminomethyl}phenyl)methyl]-6-oxopiperazine-2-carboxylate

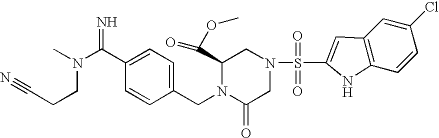

The titled compound was synthesized using a similar procedure to that described in Example 22. MS found: (M+H)⁺: 571.

### Example 100

### (2R)-4-[(5-Chloroindol-2-yl)sulfonyl]-1-[(4-{[(2-cyanoethyl)methylamino]iminomethyl}phenyl)methyl]-6-oxopiperazine-2-carboxylic acid

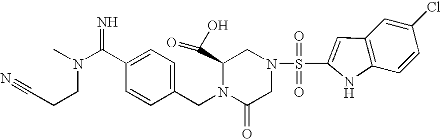

The titled compound was synthesized using a similar procedure to that described in Example 3. MS found: (M+H)⁺: 557.1.

### Example 101

### Methyl (2R)-1-({4-[(dimethylamino)iminomethyl]phenyl}methyl)-4-[(5-chloroindol-2-yl)sulfonyl]-6-oxopiperazine-2-carboxylate

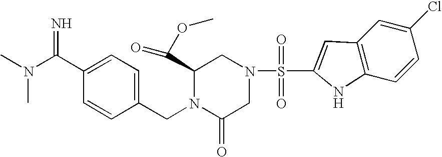

The titled compound was synthesized using a similar procedure to that described in Example 22. MS found: (M+H)⁺: 532.1

### Example 102

### (2R)-1-({4-[(Dimethylamino)iminomethyl]phenyl}methyl)4-[(5-chloroindol-2-yl)sulfonyl]-6-oxopiperazine-2-carboxylic acid

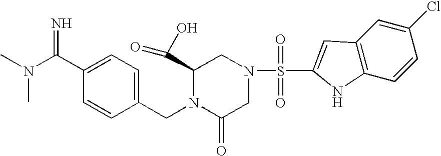

The titled compound was synthesized using a similar procedure to that described in Example 3. MS found: (M+H)⁺: 518.1.

### Example 103

### Methyl (2R)-4-[(5-chloroindol-2-yl)sulfonyl]-1-[(4-{imino[methyl(2-phenylethyl)amino]methyl}phenyl)methyl]-6-oxopiperazine-2-carboxylate

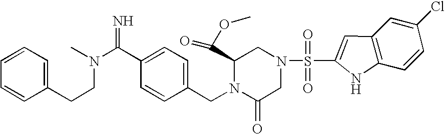

The titled compound was synthesized using a similar procedure to that described in Example 22. MS found: (M+H)⁺: 622.1.

### Example 104

### (2R)-4-[(5-Chloroindol-2-yl)sulfonyl]-1-[(4-{imino[methyl(2-phenylethyl)amino]methyl}phenyl)methyl]-6-oxopiperazine-2-carboxylic acid

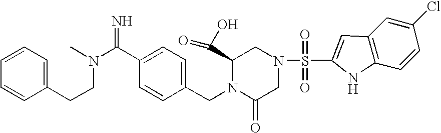

The titled compound was synthesized using a similar procedure to that described in Example 3. MS found: (M+H)⁺: 608.1.

### Example 105

### Methyl (2R)-4-[(5-chloroindol-2-yl)sulfonyl]-1-[(4-{imino[methyl(2-(2-pyridyl)ethyl)amino]methyl}phenyl)methyl]-6-oxopiperazine-2-carboxylate

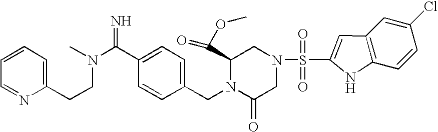

The titled compound was synthesized using a similar procedure to that described in Example 22. MS found: (M+H)⁺: 623.1.

### Example 106

### (2R)4-[(5-Chloroindol-2-yl)sulfonyl]-1-[(4-{imino[methyl(2-(2-pyridyl)ethyl)amino]methyl}phenyl)methyl]-6-oxopiperazine-2-carboxylic acid

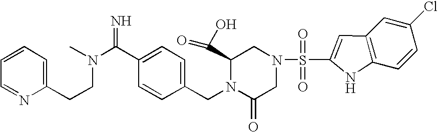

The titled compound was synthesized using a similar procedure to that described in Example 3. MS found: (M+H)⁺: 609.1.

### Example 107

### Methyl (2R)-4-[(5-chloroindol-2-yl)sulfonyl]-1-{[4-(1-methyl(2-imidazolin-2-yl))phenyl]methyl}-6-oxopiperazine-2-carboxylate

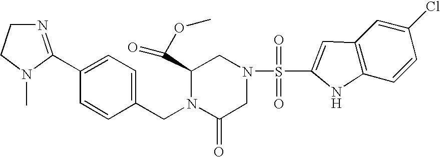

The titled compound was synthesized using a similar procedure to that described in Example 22. MS found: (M+H)⁺: 544.1.

### Example 108

### (2R)-4-[(5-Chloroindol-2-yl)sulfonyl]-1-{[4-(1-methyl(2-imidazolin-2-yl))phenyl]methyl}-6-oxopiperazine-2-carboxylic acid

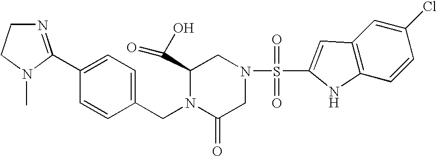

The titled compound was synthesized using a similar procedure to that described in Example 3. MS found: (M+H)⁺: 530.

### Example 109

### Methyl (2R)-4-[(5-chloroindol-2-yl)sulfonyl]-1-[(4-{imino[methyl(2-oxoethyl)amino]methyl}phenyl)methyl]-6-oxopiperazine-2-carboxylate

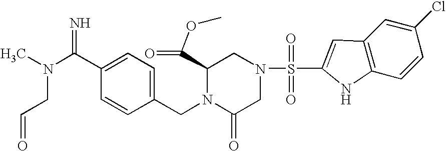

The titled compound was synthesized using a similar procedure to that described in Example 37 using compound of example 83. MS found: (M+H)⁺: 560.1.

### Example 110

### (2R)-4-[(5-Chloroindol-2-yl)sulfonyl]-1-[(4-{imino[methyl(2-oxoethyl)amino]methyl}phenyl)methyl]-6-oxopiperazine-2-carboxylic acid

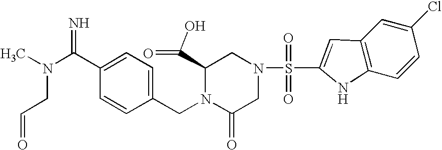

The titled compound was synthesized using a similar procedure to that described in Example 3. MS found: (M+H)⁺: 546.1.

### Example 111

### Methyl (2R)-4-[(6-chlorobenzo[b]thiophen-2-yl)sulfonyl]-1-[(4-{imino[methyl(2-oxoethyl)amino]methyl}phenyl)methyl]-6-oxopiperazine-2-carboxylate

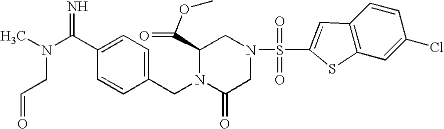

The titled compound was synthesized using a similar procedure to that described in Example 37 using compound of example 57. MS found: (M+H)⁺: 577.

### Example 112

### (2R)-4-[(6-Chlorobenzo[b]thiophen-2-yl)sulfonyl]-1-[(4-{imino[methyl(2-oxoethyl)amino]methyl}phenyl)methyl]-6-oxopiperazine-2-carboxylic acid

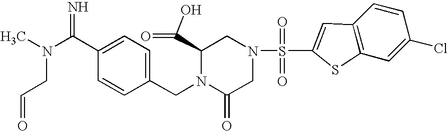

The titled compound was synthesized using a similar procedure to that described in Example 3. MS found: (M+H)⁺: 563.

### Example 113

### Methyl (2R)-1-{[4-(azetidinylazetidinylidenemethyl)phenyl]methyl}-4-[(5-chloroindol-2-yl)sulfonyl]-6-oxopiperazine-2-carboxylate

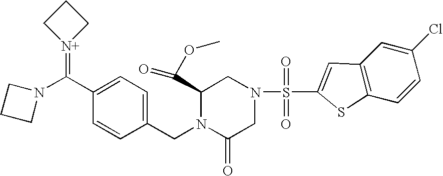

The titled compound was synthesized using a similar procedure to that described in Example 22 as a minor product in the synthesis of compound of example 85. MS found: (M+H)⁺: 585.

### Example 114

### (2R)-1-{[4-(Azetidinylazetidinylidenemethyl)phenyl]methyl}-4-[(5-chloroindol-2-yl)sulfonyl]-6-oxopiperazine-2-carboxylic acid

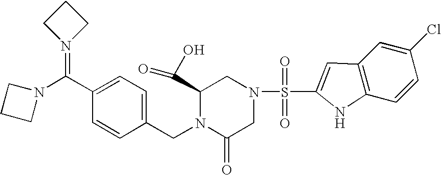

The titled compound was synthesized using a similar procedure to that described in Example 3. MS found: (M+H)⁺: 571.

### Example 115

### Methyl (2R)-1-{[4-(azetidinylazetidinylidenemethyl)phenyl]methyl}-4-[(6-chlorobenzo[b]thiophen-2-yl)sulfonyl]-6-oxopiperazine-2-carboxylate

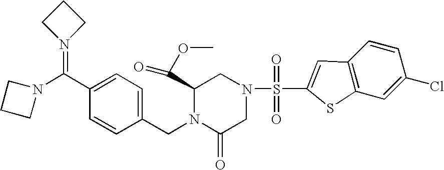

The titled compound was synthesized using a similar procedure to that described in Example 1 in the synthesis of compound of example 59 as a minor product. MS found: (M+H)⁺: 602.

### Example 116

### (2R)-1-{[4-(Azetidinylazetidinylidenemethyl)phenyl]methyl}-4-[(6-chlorobenzo[b]thiophen-2-yl)sulfonyl]-6-oxopiperazine-2-carboxylic acid

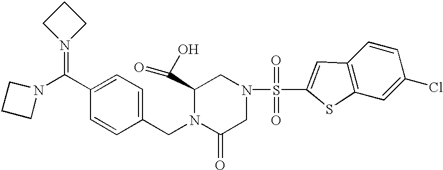

The titled compound was synthesized using a similar procedure to that described in Example 3. MS found: (M+H)⁺: 588.

### Compositions and Formulations

Compositions or formulations of the compounds of the invention are prepared for storage or administration by mixing the compound having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A.R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween®, Pluronics® or polyethyleneglycol.

Dosage formulations of the compounds of the invention to be used for therapeutic administration must be sterile. Sterility is readily accomplished by filtration through sterile membranes such as 0.2 micron membranes, or by other conventional methods. Formulations typically will be stored in lyophilized form or as an aqueous solution. The pH of the preparations of the invention typically will be between about 3 and about 11, more preferably from about 5 to about 9 and most preferably from about 7 to about 8. It will be understood that use of certain of the foregoing excipients, carriers, or stabilizers will result in the formation of cyclic polypeptide salts. While the preferred route of administration is by injection, other methods of administration are also anticipated such as intravenously (bolus and/or infusion), subcutaneously, intramuscularly, colonically, rectally, nasally or intraperitoneally, employing a variety of dosage forms such as suppositories, implanted pellets or small cylinders, aerosols, oral dosage formulations and topical formulations such as ointments, drops and dermal patches. The compounds of the invention are desirably incorporated into shaped articles such as implants which may employ inert materials such as biodegradable polymers or synthetic silicones, for example, Silastic, silicone rubber or other polymers commercially available.

The compounds of the invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compounds of the invention may also be delivered by the use of antibodies, antibody fragments, growth factors, hormones, or other targeting moieties, to which the compound molecules are coupled. The compounds of the invention may also be coupled with suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the factor Xa inhibitors of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

Therapeutic compound liquid formulations generally are placed into a container having a sterile access port, for example, an intravenous solution bag or vial having a stopper pierceable by hypodermic injection needle.

Therapeutically effective dosages may be determined by either in vitro or in vivo methods. For each particular compound of the present invention, individual determinations may be made to determine the optimal dosage required. The range of therapeutically effective dosages will naturally be influenced by the route of administration, the therapeutic objectives, and the condition of the patient. For injection by hypodermic needle, it may be assumed the dosage is delivered into the body's fluids. For other routes of administration, the absorption efficiency must be individually determined for each inhibitor by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be within the ambit of one skilled in the art. Typically, applications of compound are commenced at lower dosage levels, with dosage levels being increased until the desired effect is achieved.

A typical dosage of the compounds and compositions of the invention range from about 0.001 mg/kg to about 1000 mg/kg, preferably from about 0.01 mg/kg to about 100 mg/kg, and more preferably from about 0.10 mg/kg to about 20 mg/kg. Advantageously, the compounds of the invention may be administered several times daily. Other dosage regimens may also be useful (e.g. single daily dose and/or continuous infusion).

Typically, about 0.5 to about 500 mg of a compound or mixture of compounds of the invention, as the free acid or base form or as a pharmaceutically acceptable salt, is compounded with a physiologically acceptable vehicle, carrier, excipient, binder, preservative, stabilizer, dye, flavor, etc., as called for by accepted pharmaceutical practice. The amount of active ingredient in these compositions is such that a suitable dosage in the range indicated is obtained.

Typical adjuvants which may be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. When a dosage form is a capsule, in addition to the above materials it may also contain a liquid carrier such as water, saline, a fatty oil. Other materials of various types may be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

The preferred compounds of the present invention are characterized by their ability to inhibit thrombus formation with acceptable effects on classical measures of coagulation parameters, platelets and platelet function, and acceptable levels of bleeding complications associated with their use. Conditions characterized by undesired thrombosis would include those involving the arterial and venous vasculature.

With respect to the coronary arterial vasculature, abnormal thrombus formation characterizes the rupture of an established atherosclerotic plaque which is the major cause of acute myocardial infarction and unstable angina, as well as also characterizing the occlusive coronary thrombus formation resulting from either thrombolytic therapy or percutaneous transluminal coronary angioplasty (PTCA).

With respect to the venous vasculature, abnormal thrombus formation characterizes the condition observed in patients undergoing major surgery in the lower extremities or the abdominal area who often suffer from thrombus formation in the venous vasculature resulting in reduced blood flow to the affected extremity and a predisposition to pulmonary embolism. Abnormal thrombus formation further characterizes disseminated intravascular coagulopathy commonly occurs within both vascular systems during septic shock, certain viral infections and cancer, a condition wherein there is rapid consumption of coagulation factors and systemic coagulation which results in the formation of life-threatening thrombi occurring throughout the microvasculature leading to widespread organ failure.

The compounds of the invention are useful for the treatment or prophylaxis of those diseases which involve the production and/or action of factor Xa/prothrombinase complex. The compounds of this present invention, selected and used as disclosed herein, find utility as a diagnostic or therapeutic agent for preventing or treating a condition in a mammal characterized by undesired thrombosis or a disorder of coagulation. Disease states treatable or preventable by the administration of compounds of the invention include, without limitation, occlusive coronary thrombus formation resulting from either thrombolytic therapy or percutaneous transluminal coronary angioplasty, thrombus formation in the venous vasculature, disseminated intravascular coagulopathy, the treatment of reocclusion or restenosis of reperfused coronary arteries, thromboembolic complications of surgery and peripheral arterial occlusion, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

Accordingly, the invention provides a method for preventing or treating a condition in a mammal characterized by undesired thrombosis which administers to a mammal a therapeutically effective amount of a compound of the invention, as described herein. Conditions for prevention or treatment include, for example, (a) the treatment or prevention of any thrombotically mediated acute coronary syndrome including myocardial infarction, unstable angina, refractory angina, occlusive coronary thrombus occurring post-thrombolytic therapy or post-coronary angioplasty, (b) the treatment or prevention of any thrombotically mediated cerebrovascular syndrome including embolic stroke, thrombotic stroke or transient ischemic attacks, (c) the treatment or prevention of any thrombotic syndrome occurring in the venous system including deep venous thrombosis or pulmonary embolus occurring either spontaneously or in the setting of malignancy, surgery or trauma, (d) the treatment or prevention of any coagulopathy including disseminated intravascular coagulation (including the setting of septic shock or other infection, surgery, pregnancy, trauma or malignancy and whether associated with multi-organ failure or not), thrombotic thrombocytopenic purpura, thromboangiitis obliterans, or thrombotic disease associated with heparin induced thrombocytopenia, (e) the treatment or prevention of thrombotic complications associated with extracorporeal circulation (e.g. renal dialysis, cardiopulmonary bypass or other oxygenation procedure, plasmapheresis), (f) the treatment or prevention of thrombotic complications associated with instrumentation (e.g. cardiac or other intravascular catheterization, intra-aortic balloon pump, coronary stent or cardiac valve), and (g) those involved with the fitting of prosthetic devices.

Anticoagulant therapy is also useful to prevent coagulation of stored whole blood and to prevent coagulation in other biological samples for testing or storage. Thus the compounds of the invention can be added to or contacted with any medium containing or suspected to contain factor Xa and in which it is desired that blood coagulation be inhibited, e.g., when contacting the mammal's blood with material such as vascular grafts, stents, orthopedic prostheses, cardiac stents, valves and prostheses, extra corporeal circulation systems and the like.

Thus, the compounds of the invention also find utility in a method for inhibiting the coagulation of biological samples by administration of a compound of the invention.

### Biological Activity Examples

Evaluation of the compounds of the invention is guided by in vitro protease activity assays (see below) and in vivo studies to evaluate antithrombotic efficacy, and effects on hemostasis and hematological parameters.

The compounds of the present invention are dissolved in buffer to give solutions containing concentrations such that assay concentrations range from about 0 to 100 μM. In the assays for thrombin, prothrombinase and factor Xa, a synthetic chromogenic substrate is added to a solution containing test compound and the enzyme of interest and the residual catalytic activity of that enzyme is determined spectrophotometrically. The IC₅₀ of a compound is determined from the substrate turnover. The IC₅₀ is the concentration of test compound giving 50% inhibition of the substrate turnover. The compounds of the present invention desirably have an IC₅₀ of less than about 500 nM in the factor Xa assay, preferably less than about 200 nM, and more preferred compounds have an IC₅₀ of about 100 nM or less in the factor Xa assay. The compounds of the present invention desirably have an IC₅₀ of less than about 4.0 μM in the prothrombinase assay, preferably less than 200 nM, and more preferred compounds have an IC₅₀ of about 10 nM or less in the prothrombinase assay. The compounds of the present invention desirably have an IC₅₀ of greater than about 1.0 μM in the thrombin assay, preferably greater than about 10.0 μM, and more preferred compounds have an IC₅₀ of greater than about 100.0 μM in the thrombin assay.

Amidolytic Assays for Determining Protease Inhibition Activity

The factor Xa and thrombin assays are performed at room temperature, in 0.02 M Tris.HCl buffer, pH 7.5, containing 0.15 M NaCl. The rates of hydrolysis of the para-nitroanilide substrate S-2765 (Chromogenix) for factor Xa, and the substrate Chromozym TH (Boehringer Mannheim) for thrombin following preincubation of the enzyme with inhibitor for 5 minutes at room temperature, and were determined using the Softmax 96-well plate reader (Molecular Devices), monitored at 405 nm to measure the time dependent appearance of p-nitroaniline.

The prothrombinase inhibition assay is performed in a plasma free system with modifications to the method described by Sinha, U. et al., Thromb. Res., 75, 427-436 (1994). Specifically, the activity of the prothrombinase complex is determined by measuring the time course of thrombin generation using the p-nitroanilide substrate Chromozym TH. The assay consists of preincubation (5 minutes) of selected compounds to be tested as inhibitors with the complex formed from factor Xa (0.5 nM), factor Va (2 nM), phosphatidyl serine:phosphatidyl choline (25:75, 20 μM) in 20 mM Tris.HCl buffer, pH 7.5, containing 0.15 M NaCl, 5 mM CaCl₂ and 0.1% bovine serum albumin. Aliquots from the complex-inhibitor mixture are added to prothrombin (1 nM) and Chromozym TH (0.1 mM). The rate of substrate cleavage is monitored at 405 nm for two minutes. Eight different concentrations of inhibitor are assayed in duplicate. A standard curve of thrombin generation by an equivalent amount of untreated complex are used for determination of percent inhibition.

Antithrombotic Efficacy in a Rabbit Model of Venous Thrombosis

A rabbit deep vein thrombosis model as described by Hollenbach, S. et al., Thromb. Haemost. 71, 357-362 (1994), is used to determine the in-vivo antithrombotic activity of the test compounds. Rabbits are anesthetized with I.M. injections of Ketamine, Xylazine, and Acepromazine cocktail. A standardized protocol consists of insertion of a thrombogenic cotton thread and copper wire apparatus into the abdominal vena cava of the anesthetized rabbit. A non-occlusive thrombus is allowed to develop in the central venous circulation and inhibition of thrombus growth is used as a measure of the antithrombotic activity of the studied compounds. Test agents or control saline are administered through a marginal ear vein catheter. A femoral vein catheter is used for blood sampling prior to and during steady state infusion of test compound. Initiation of thrombus formation begins immediately after advancement of the cotton thread apparatus into the central venous circulation. Test compounds are administered from time =30 min to time =150 min at which the experiment is terminated. The rabbits are euthanized and the thrombus excised by surgical dissection and characterized by weight and histology. Blood samples are analyzed for changes in hematological and coagulation parameters.

Effects of Compounds in Rabbit Venous Thrombosis Model

Administration of compounds in the rabbit venous thrombosis model demonstrates antithrombotic efficacy at the higher doses evaluated. There are no significant effects of the compound on the aPTT and PT prolongation with the highest dose (100 μg/kg+2.57 μg/kg/min). Compounds have no significant effects on hematological parameters as compared to saline controls. All measurements are an average of all samples after steady state administration of vehicle or (D)-Arg-Gly-Arg-thiazole. Values are expressed as mean±SD.

Without further description, it is believed that one of ordinary skill in the art can, using the preceding description and the following illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

What is claimed is:

1. A compound of the formulae (I) or (II): 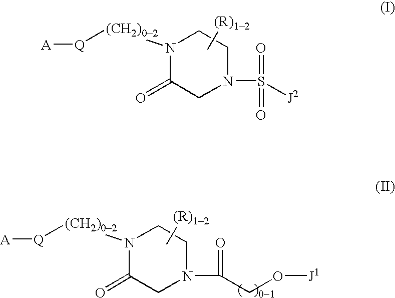
wherein:
A is a member selected from the group consisting of: 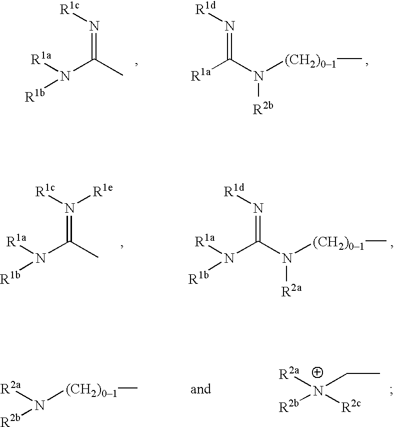
R^{1a}, R^{1b}, R^{1d}, and R^{1e} are each independently a H, C₁₋₆ alkyl, C₂₋₆ alkenyl, C₂₋₆ alkynyl, C₃₋₈cycloalkyl, aryl, —C₁₋₆alkylaryl, heterocyclyl, —C₁₋₆alkylheterocyclyl, —(CH₂)₁₋₆OH, —(CH₂)₁₋₆OC₁₋₆ alkyl, —(CH₂)₁₋₆NH₂, —(CH₂)₁₋₆NHC₁₋₆ alkyl, —(CH₂)₁₋₆N(C₁₋₆ alkyl)₂, —(CH₂)₁₋₆CHNH(COOH), —(CH₂)₁₋₆NHC(═O)C₁₋₆ alkyl, —(CH₂)₁₋₆CHO, —(CH₂)₁₋₆C(═O)OH, —(CH₂)₁₋₆C(═O)OC₁₋₆alkyl, or —(CH₂)₁₋₆C(═O)NH₂; wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide; or R^{1a} and R^{1b} or R^{1a} and R^{1c} or R^{1a} and R^{1d} or R^{1d} and R^{1e} taken together with the nitrogen atom to which they are each attached can form a substituted or unsubstituted 3 to 8 membered heterocyclic or heteroaromatic amine group which, optionally, contains at least one other heteroatom of N, O or S;
wherein R^{1a}, R^{1b}, R^{1d}, or R^{1e} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;
R^{1c} is H, C₁₋₆alkyl or C₃₋₈cycloalkyl;
R^{2a}, R^{2b} and R^{2c} are each independently a H, C₁₋₆ alkyl, C₂₋₆ alkenyl, C₂₋₆ alkynyl, C₃₋₈cycloalkyl, aryl, —C₁₋₆alkylaryl, heterocyclyl, —C₁₋₆alkylheterocyclyl, —(CH₂)₁₋₆OH, —(CH₂)₁₋₆OC₁₋₆ alkyl, —(CH₂)₁₋₆NH₂, —(CH₂)₁₋₆NHC₆ alkyl, —(CH₂)₁₋₆N(C₁₋₆ alkyl)₂, —(CH₂)₁₋₆CHNH(COOH), —(CH₂)₁₋₆NHC(═O)C₁₋₆ alkyl, —(CH₂)₁₋₆CHO, —(CH₂)₁₋₆C(═O)OH, —(CH₂)₁₋₆C(═O)OC₁₋₆alkyl, or —(CH₂)₁₋₆C(═O)NH₂; wherein R^{2a}, R^{2b} and R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide; or R^{2a} and R^{2b} or R^{1a}, as set forth above, and R^{2a} or R^{1a}, as set forth above, and R^{2b} taken together with the nitrogen atom to which they are each attached can form a substituted or unsubstituted 3 to 8 membered heterocyclic or heteroaromatic amine group which, optionally, contains at least one other heteroatom of N, O or S; wherein R^{2a}, R^{2b} or R^{2c} is optionally substituted with at least one of halo, alkyl, alkylideneamine, arylidenamine, cyano, hydroxy, alkoxy, amino, amidino, guanidino, imino, amido, acid, ester, keto, aldehyde, dioxolane, furanyl, piperidinyl, piperazinyl, pyrrolidinyl, aryl, morpholinyl, and thiomorpholinyldioxide;
R is, in each occurrence, independently, H, —C₁₋₆alkyl, —C₃₋₈cycloalkyl, —C₀₋₆alkyl-OC₁₋₆alkyl, —C₀₋₆alkyl-O(CH₂)₁₋₄—COOH, —C₀₋₆alkyl-O(CH₂)₁₋₄—C(═O)OC₁-C₆alkyl, —C₀₋₆alkylCOOH, —C₀₋₆alkylCO₂C₁₋₆alkyl, —C₀₋₆alkylOC₁₋₆alkyl, —C₁₋₆alkylOH, —C₀₋₆alkylCONH₂, —C₀₋₆alkylCONHC₀₋₆alkyl, —C₀₋₆alkylCON(C₀₋₆alky)₂, —C₀₋₆alkylCON(CH₂)₂₋₆, —C₀₋₆alkylCON(CH₂CH₂)₂O, —C₀₋₆alkylCON(CH₂CH₂)₂SO₂—C₀₋₆alkylCONHaryl, —C₀₋₆alkylNH₂, —C₀₋₆alkylNH(C₁₋₆alkyl) or —C₀₋₆alkylN(C₁₋₆alkyl)₂.
Q is a member selected from the group consisting of: 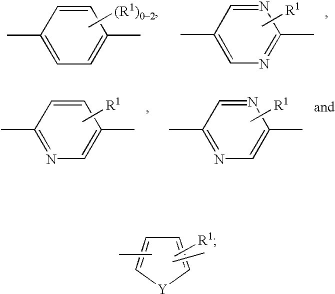
Y is S;
R¹ is H, —Cl, —Br, —I, —F, —OCF₃, alkyl, hydroxy, alkoxy, amino, thiol, thioalkyl, thioaryl, or piperizinyl;
J¹ is a member selected from the group consisting of: 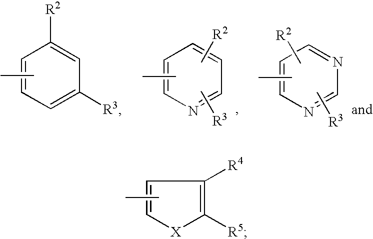
X is O or S;
R² is H, —Cl, —Br, —I, —F or —OC₁₋₆alkyl;
R³ is H, —Cl, —Br, —I, —F, —OC₁₋₆alkyl, —NHC₁₋₆acyl, —NO₂, —NHSO₂C₁₋₄alkyl, —CN, —NH₂, —CONH₂, —SO₂C₁₋₆alkyl, —SO₂NH₂, —CO₂C₁₋₆alkyl or —O(CH₂)₁₋₄COOH;
R⁴ and R⁵ are each independently H, —Cl, —Br, —I, —F or —OC₁₋₆alkyl;
J² is a member selected from the group consisting of: 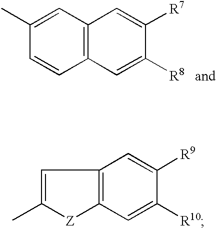
Z is —NR⁶—, —O— or —S—;
R⁶ is H, C₁₋₆alkyl or C₃₋₈cycloalkyl;
R⁷ and R⁸ are independently H, —Cl, —Br, —I or —F, where at least one of R⁷ and R⁸ is not hydrogen; and
R⁹ and R¹⁰ are independently H, —Cl, —Br, —I or —F, where at least one of R⁹ and R¹⁰ is not hydrogen;
and all pharmaceutically acceptable isomers, salts, hydrates, solvates and prodrug derivatives thereof.

2. A compound of claim 1, wherein:
A is a member selected from the group consisting of: 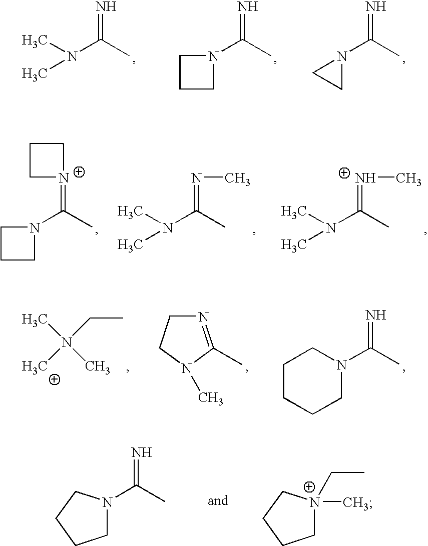
R is, in each occurrence, independently, H, —C₁₋₆alkyl, —C₃₋₈cycloalkyl, —C₀₋₆alkyl-OC₁₋₆alkyl, —C₀₋₆alkyl-O(CH₂)₁₋₄—COOH, —C₀₋₆alkyl-O(CH₂)₁₋₄—C(═O)OC₁-C₆alkyl, —C₀₋₆alkylCOOH, —C₀₋₆alkylCO₂C₁₋₆alkyl, —C₀₋₆alkylOC₁₋₆alkyl, —C₁₋₆alkylOH, —C₀₋₆alkylCONH₂, —C₀₋₆alkylCONHC₀₋₆alkyl, —C₀₋₆alkylCON(C₀₋₆alkyl)₂, —C₀₋₆alkylCON(CH₂)₂₋₆, —C₀₋₆alkylCON(CH₂CH₂)₂O, —C₀₋₆alkylCON(CH₂CH₂)₂SO₂—C₀₋₆alkylCONHaryl, —C₀₋₆alkylNH₂, —C₀₋₆alkylNH(C₁₋₆alkyl) or —C₀₋₆alkylN(C₁₋₆alkyl)₂.
R¹ is H, —Cl, —Br, —I or —F, —OCF₃, —OMe, NH₂, NHMe, NHMe₂, —NHCOMe, —NHSO₂Me; and
R³ is H, —Cl, —Br, —I, —F, —OC₁₋₆alkyl, —NHC₁₋₆acyl, —NO₂, —NHSO₂C₁₋₄alkyl, —CN or —O(CH₂)₁₋₄—COOH.

3. A compound of claim 1, wherein:
A is a member selected from the group consisting of: 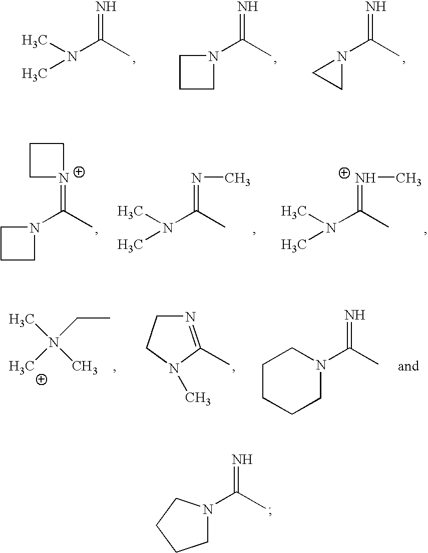
R is, in each occurrence, independently, H, —C₁₋₆alkyl, —C₃₋₈cycloalkyl, —CO₆alkyl-OC₁₋₆alkyl, —C₀₋₆alkyl-O(CH₂)₁₋₄—COOH, —C₀₋₆alkyl-O(CH₂)₁₋₄—C(═O)OC₁-C₆alkyl, —C₀₋₆alkylCOOH, —C₀₋₆alkylCO₂C₁₋₆alkyl, —C₀₋₆alkylOC₁₋₆alkyl, —C₁₋₆alkylOH, —C₀₋₆alkylCONH₂, —C₀₋₆alkylCONHC₀₋₆alkyl, —C₀₋₆alkylCON(C₀₋₆alky)₂, —C₀₋₆alkylCON(CH₂)₂₋₆, —C₀₋₆alkylCON(CH₂CH₂)₂O, —C₀₋₆alkylCON(CH₂CH₂)₂SO₂—C₀₋₆alkylCONHaryl, —C₀₋₆alkylNH₂, —C₀₋₆alkylNH(C₁₋₆alkyl) or —C₀₋₆alkylN(C₁₋₆alkyl)₂;
R¹ is H, —Cl, —Br, —I or —F, —OMe, NH2, NHMe, NHMe₂, —NHCOMe, —NHSO₂Me;
J¹ is a member selected from the group consisting of: 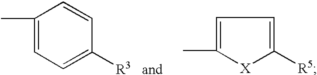
X is O or S;
R³ is H, —Cl, —Br, —I or —F;
R⁵ is H, —Cl, —Br, —I or —F;
J² is a member selected from the group consisting of: 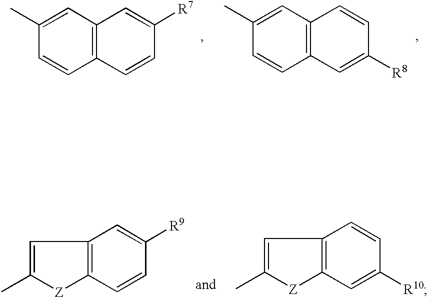
Z is —NR⁶—, —O— or —S—;
R⁶ is a H, C₁₋₆ alkyl or C₃₋₈ cycloalkyl;
R⁷ and R⁸ are each independently —Cl, —Br, —I or —F; and
R⁹ and R¹⁰ are each independently —Cl, —Br, —I or —F.

4. A compound of claim 1 of formula (I) having the following structure: 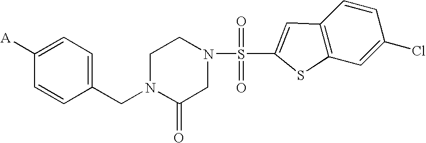
wherein:
A is a member selected from the group consisting of:

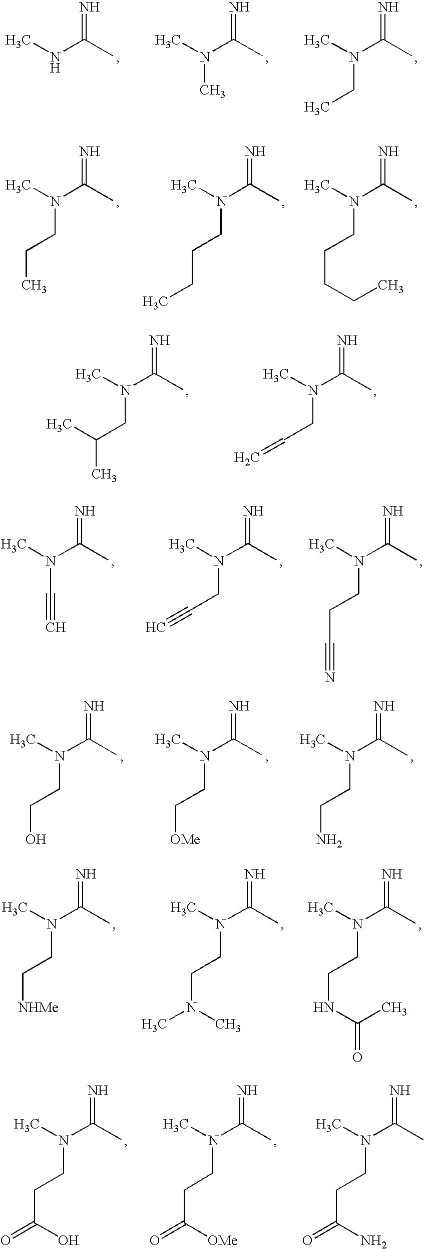

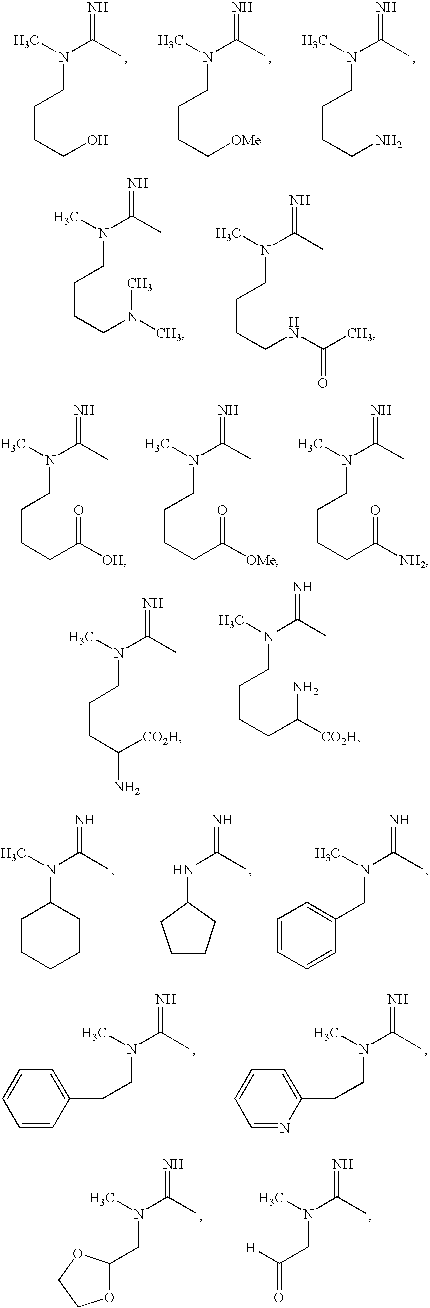

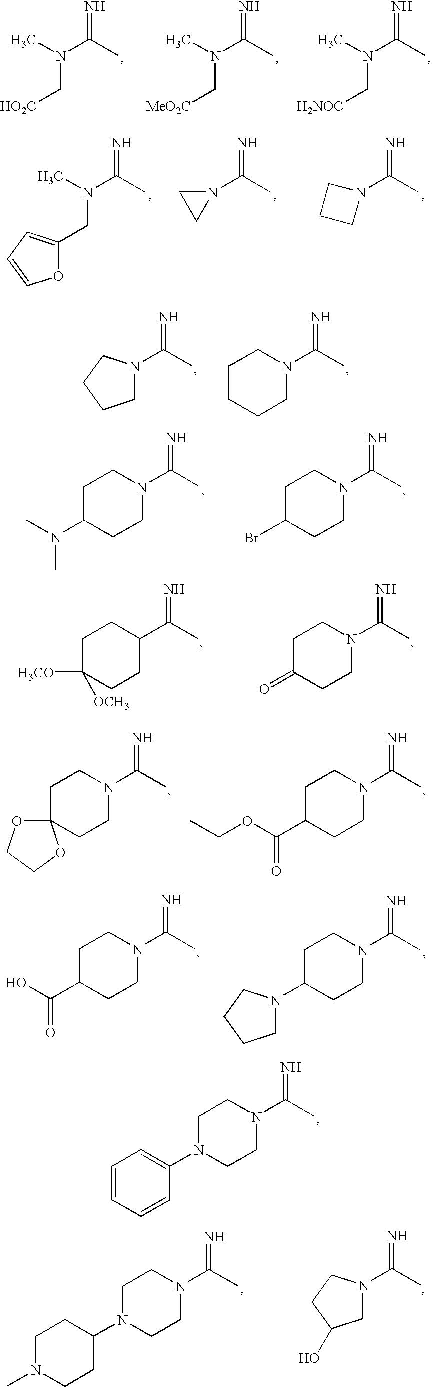

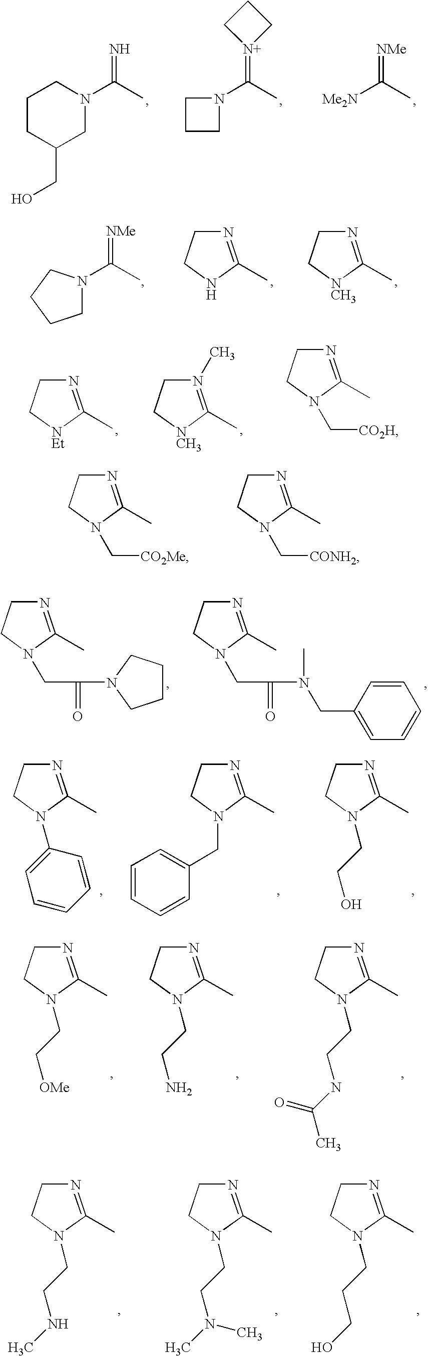

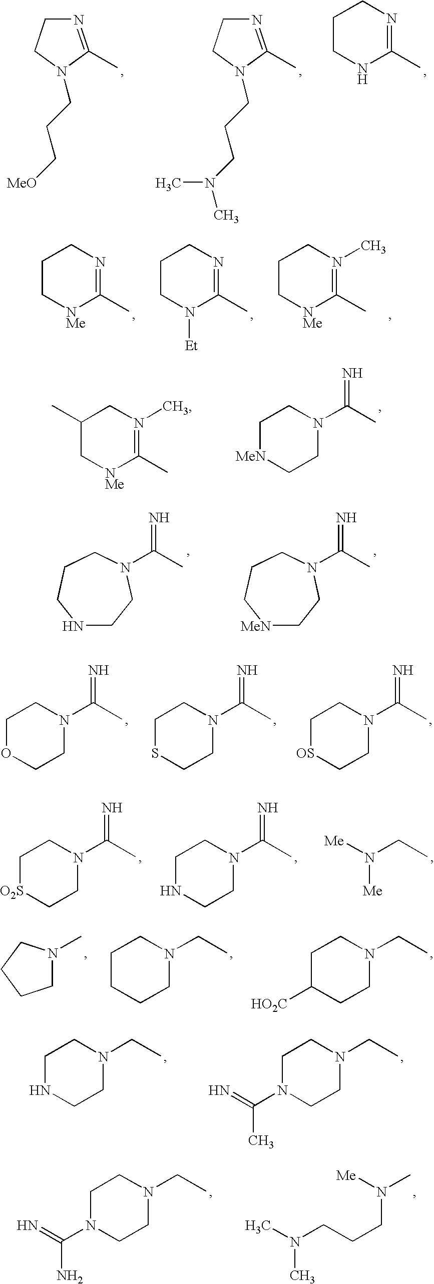

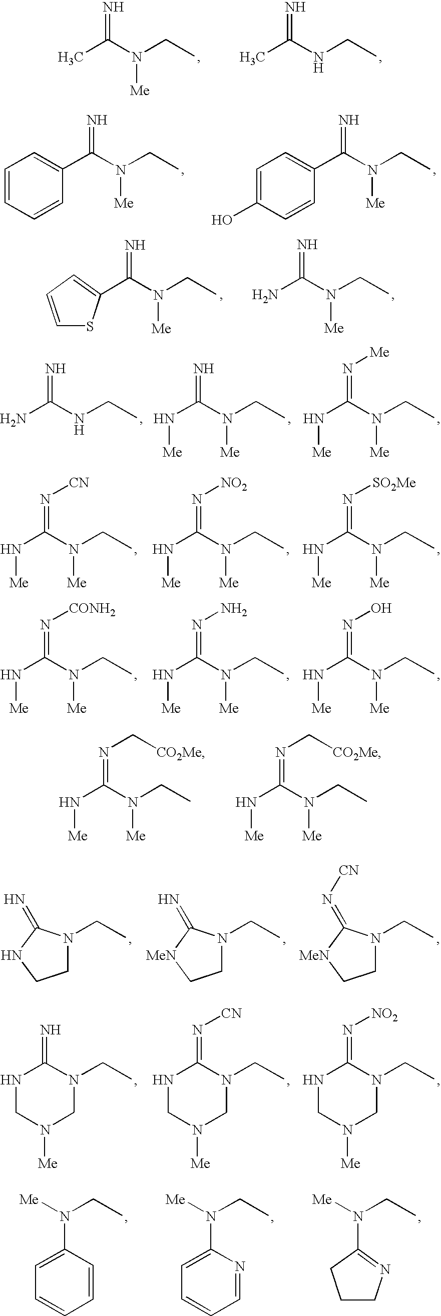

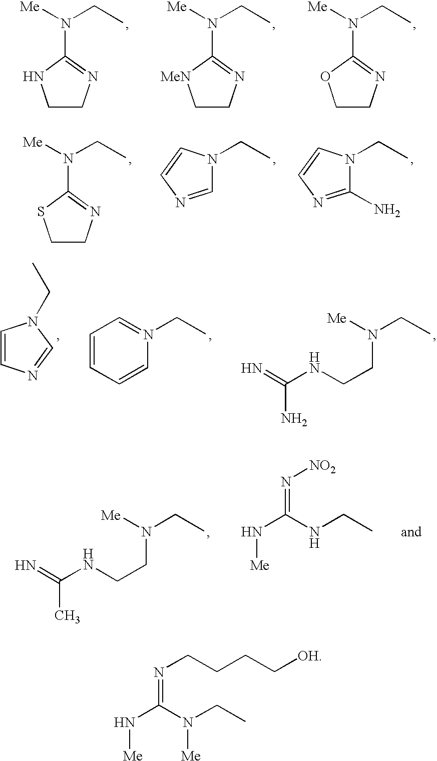

5. A compound of claim 1 of formula (I) having the following structure: 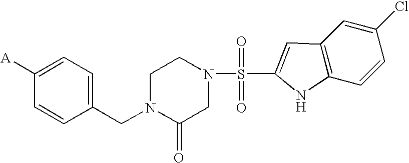
wherein:
A is a member selected from the group consisting of:

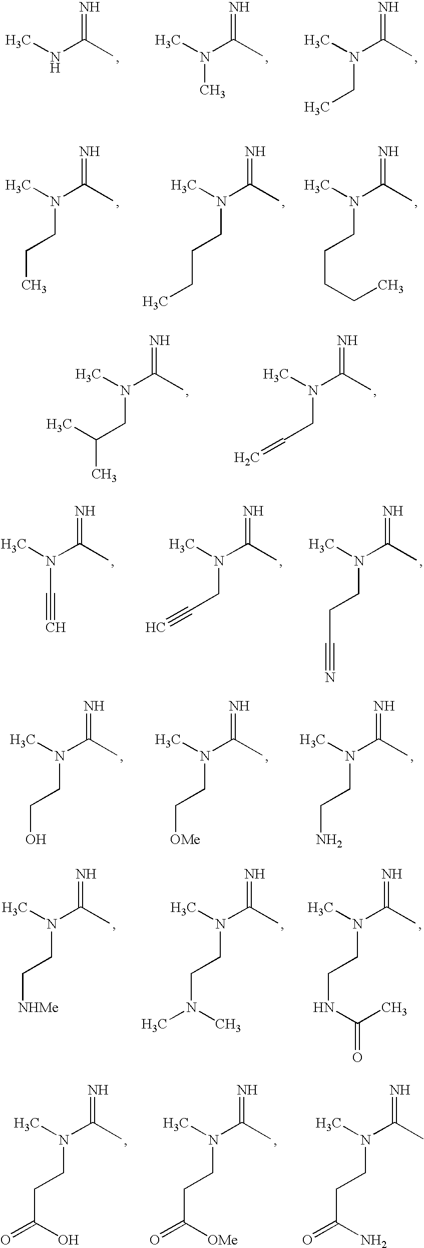

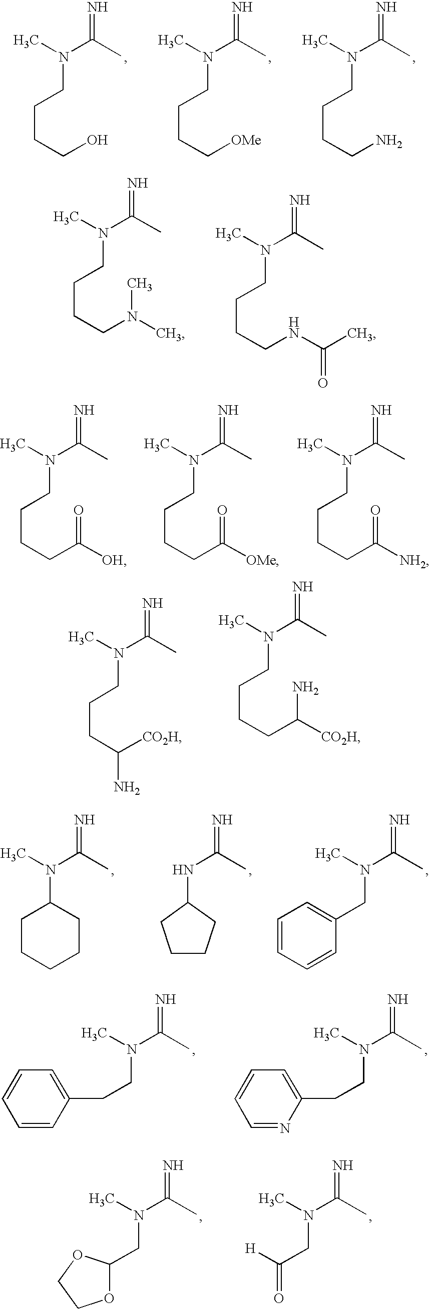

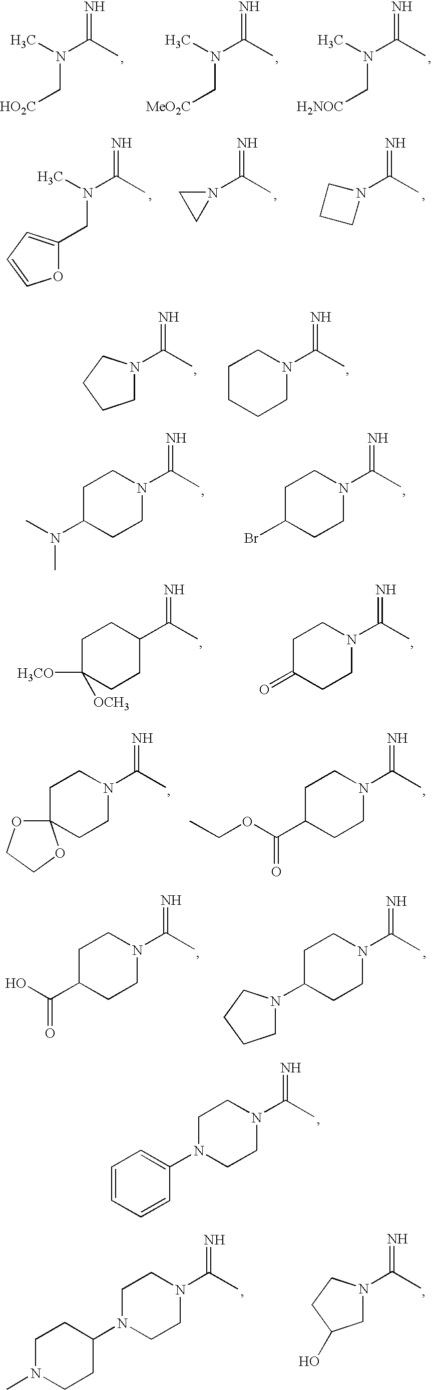

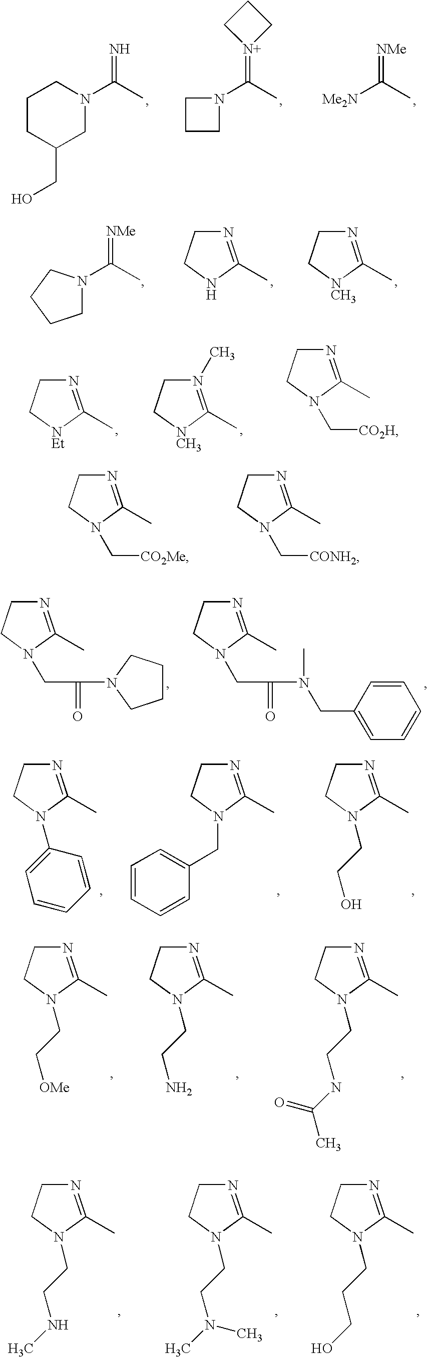

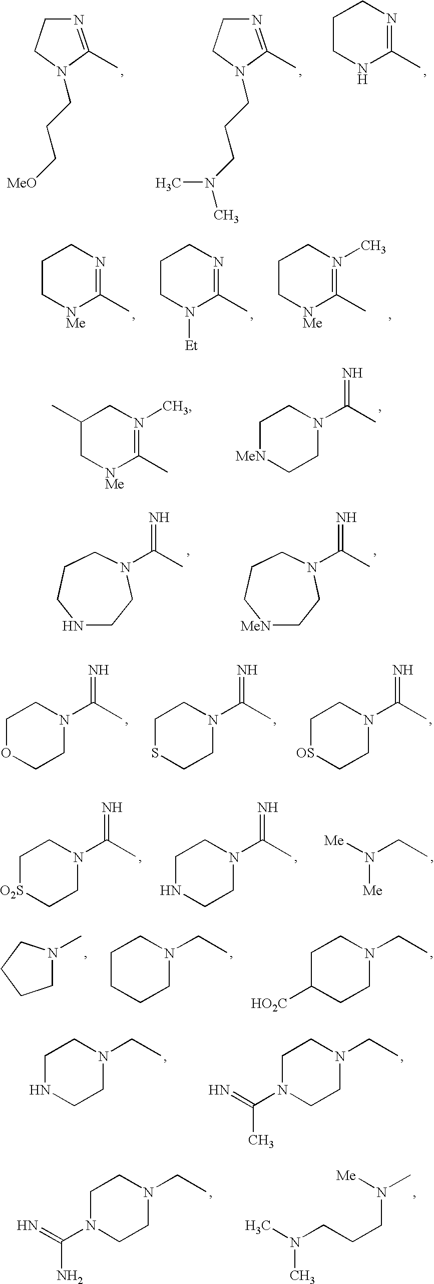

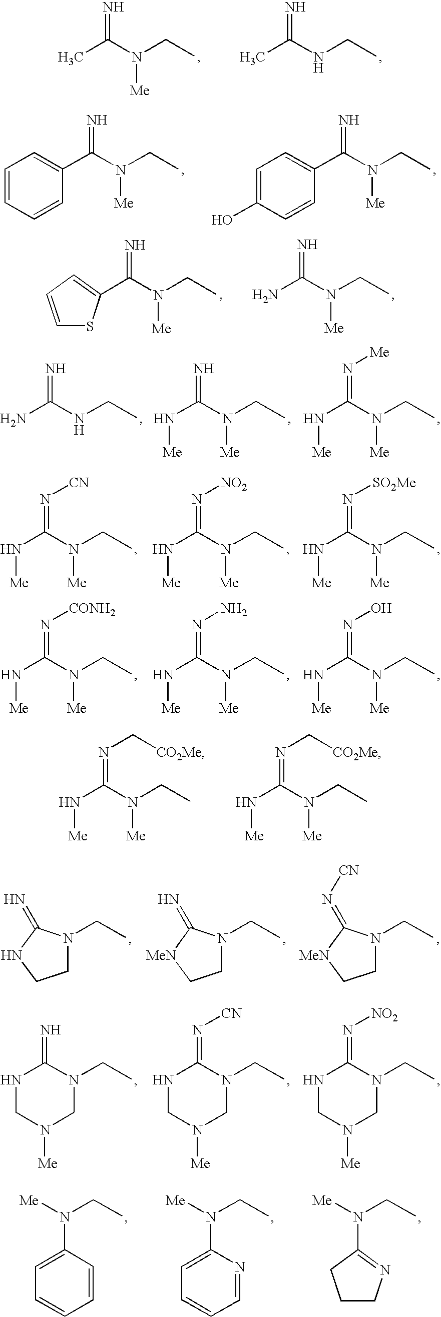

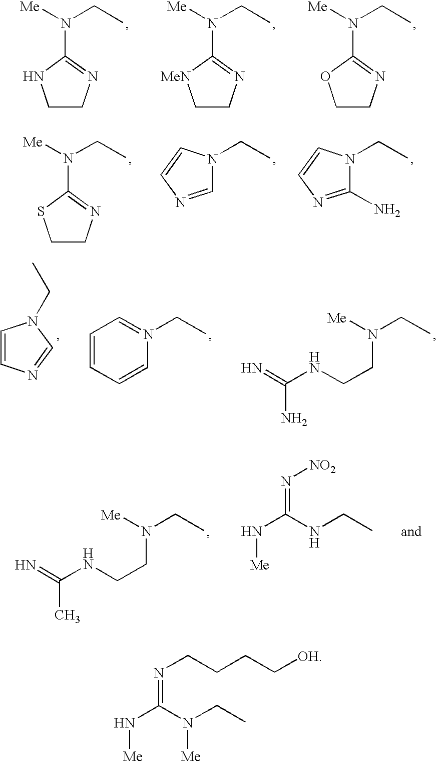

6. A compound of claim 1 of formula (I) having the following structure: 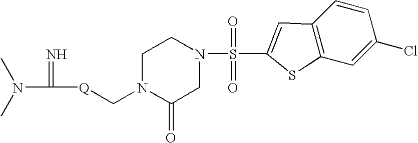
wherein:
Q is a member selected from the group consisting of:

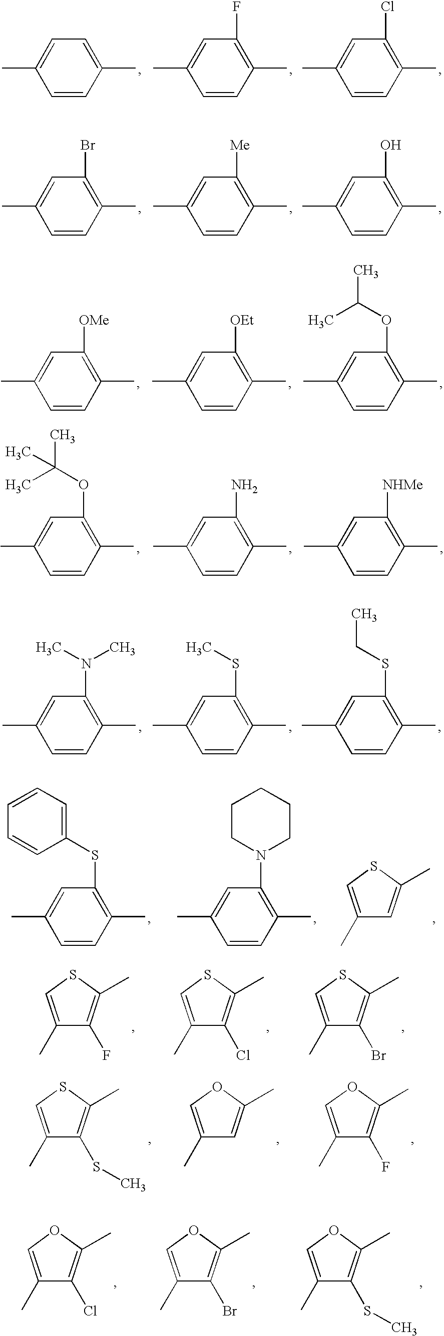

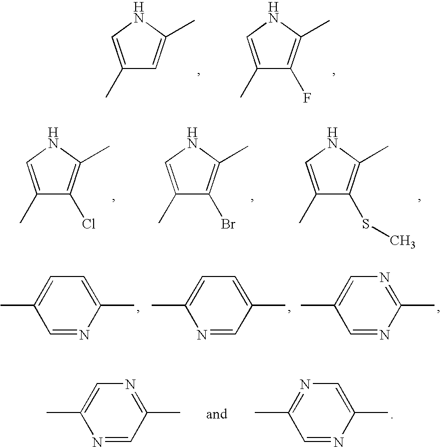

7. A compound of claim 1 of formula (I) having the following structure: 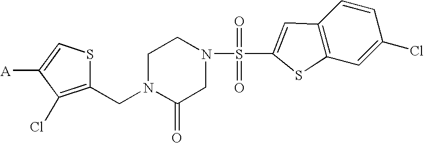
wherein:
A is a member selected from the group consisting of:

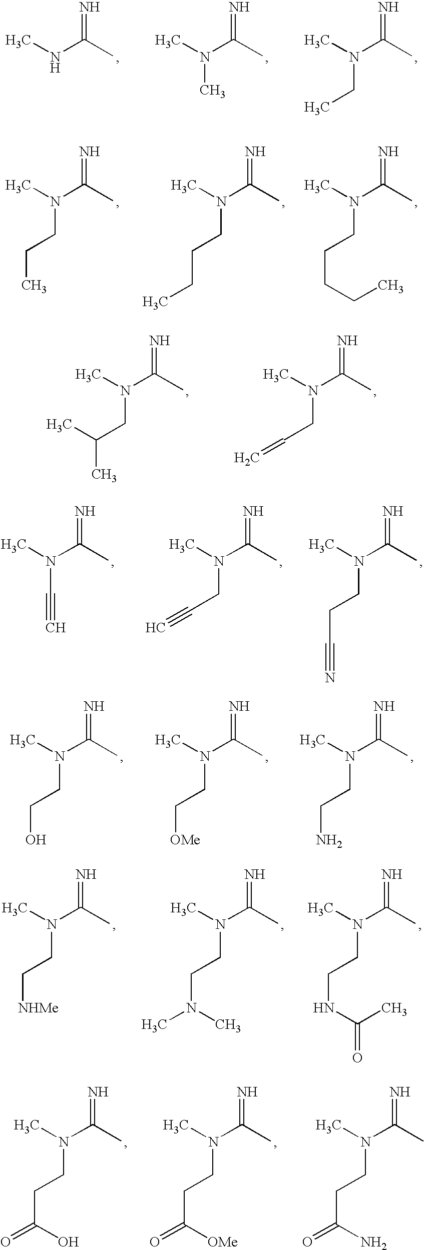

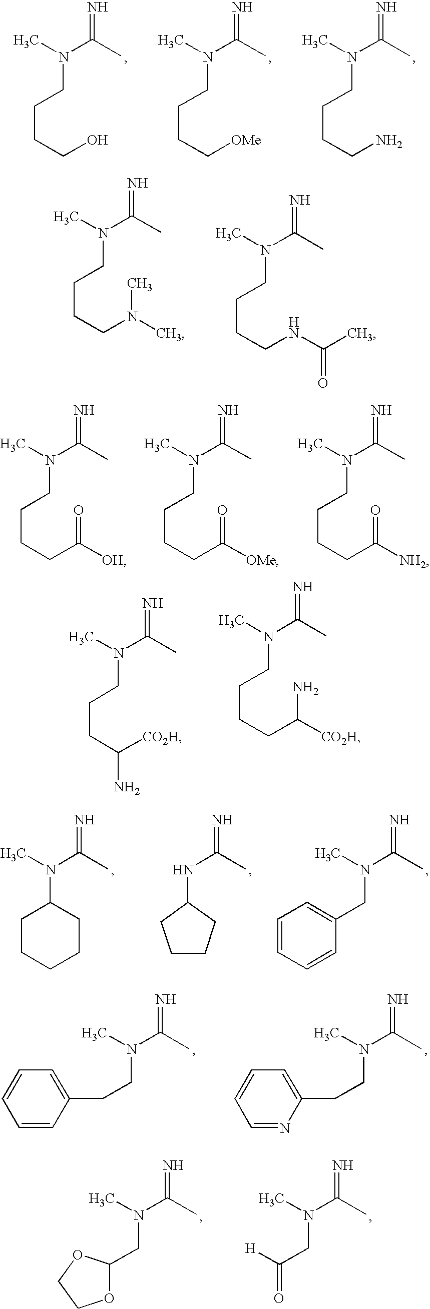

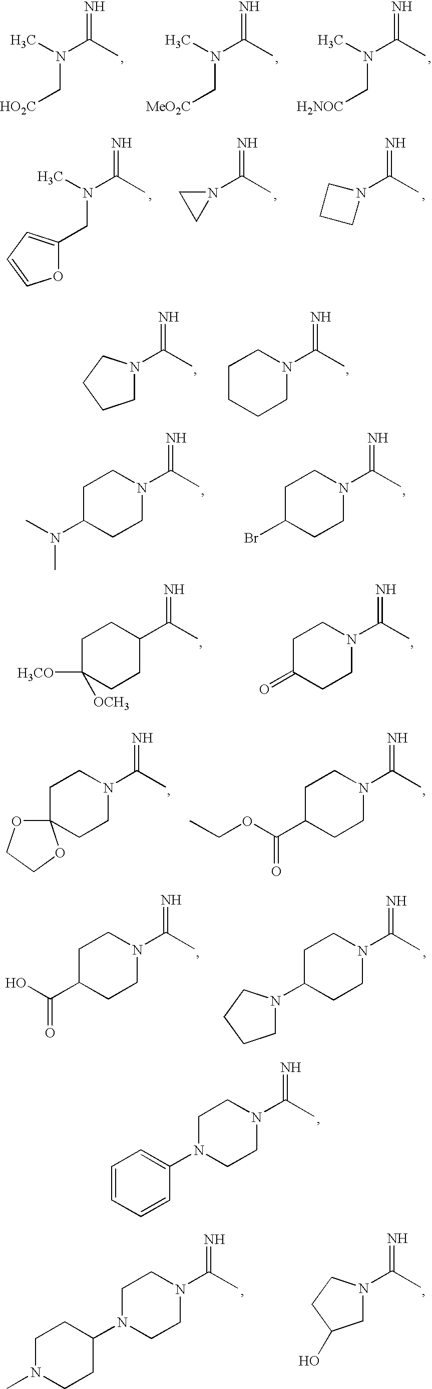

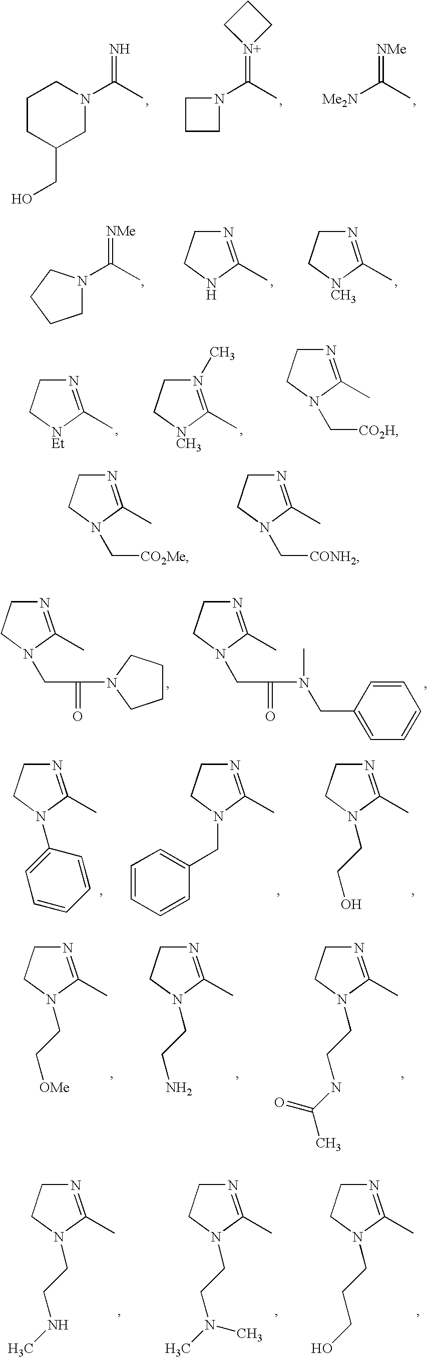

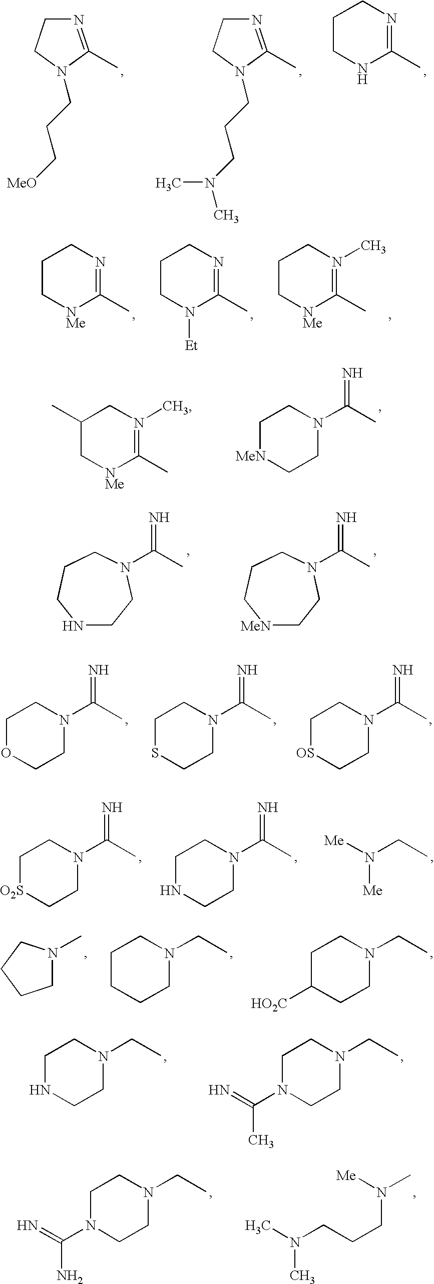

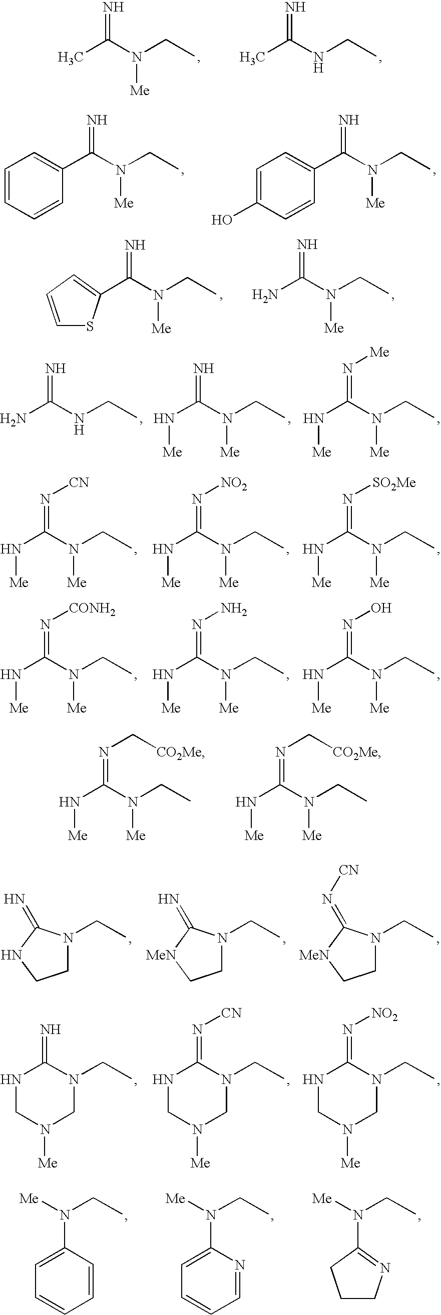

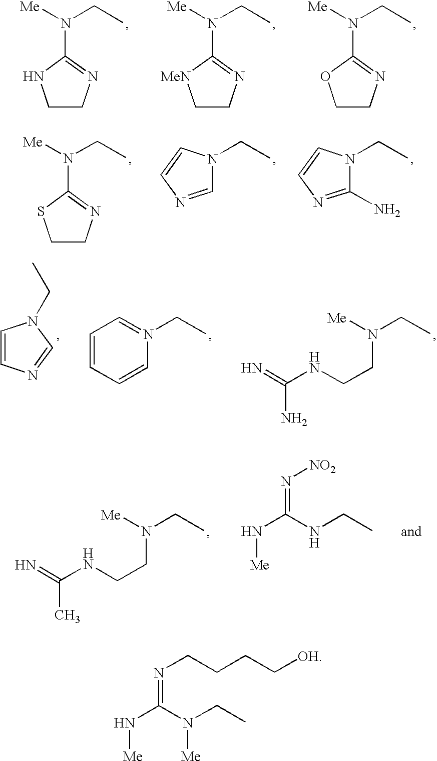

8. A compound of claim 1 of formula (I) having the following structure: 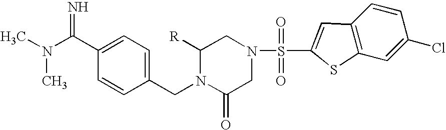
wherein:
R is independently selected from the group consisting of:
H, —CO₂H, —CO₂Me, —CONH₂, —CONHMe, —CONHMe₂, —CON(CH₂)₄, —CON(CH₂)₅, —CH₂OH, —CH₂OMe, —CH₂CO₂H, —CH₂CO₂Me, —CH₂CONH₂, —CH₂CH₂OH, —CH₂CH₂OMe, —CH₂NH₂, —CH₂N(Me)₂, and —CH₃,

9. A compound of claim 1 of formula (I) having the following structure: 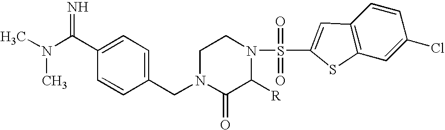
wherein:
R is independently selected from the group consisting of:
H, —CO₂H, —CO₂Me, —CONH₂, —CONHMe, —CONHMe₂, —CON(CH₂)₄, —CON(CH₂)₅, —CH₂OH, —CH₂OMe, —CH₂CO₂H, —CH₂CO₂Me, —CH₂CONH₂, —CH₂CH₂OH, —CH₂CH₂OMe, —CH₂NH₂, —CH₂N(Me)₂, and —CH₃,

10. A compound of claim 1 of formula (I) having the following structure: 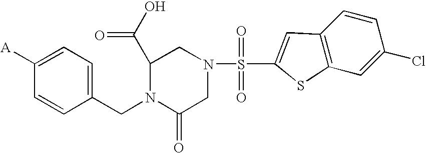
wherein:
A is a member selected from the group consisting of:

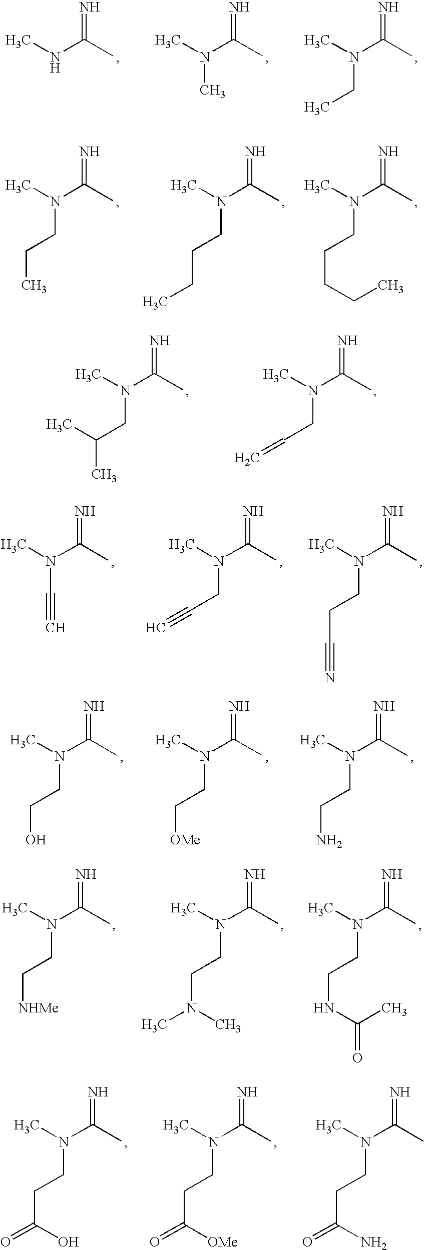

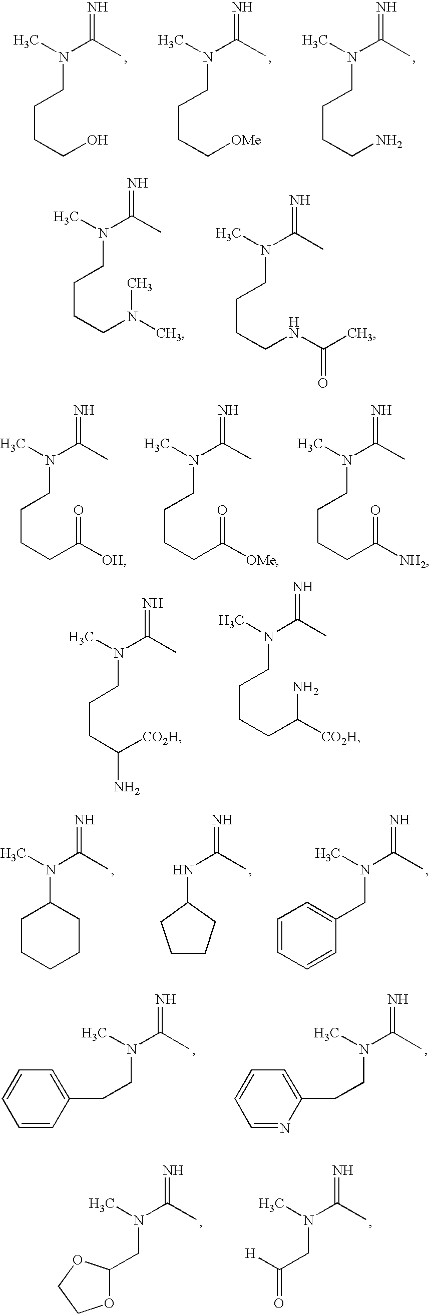

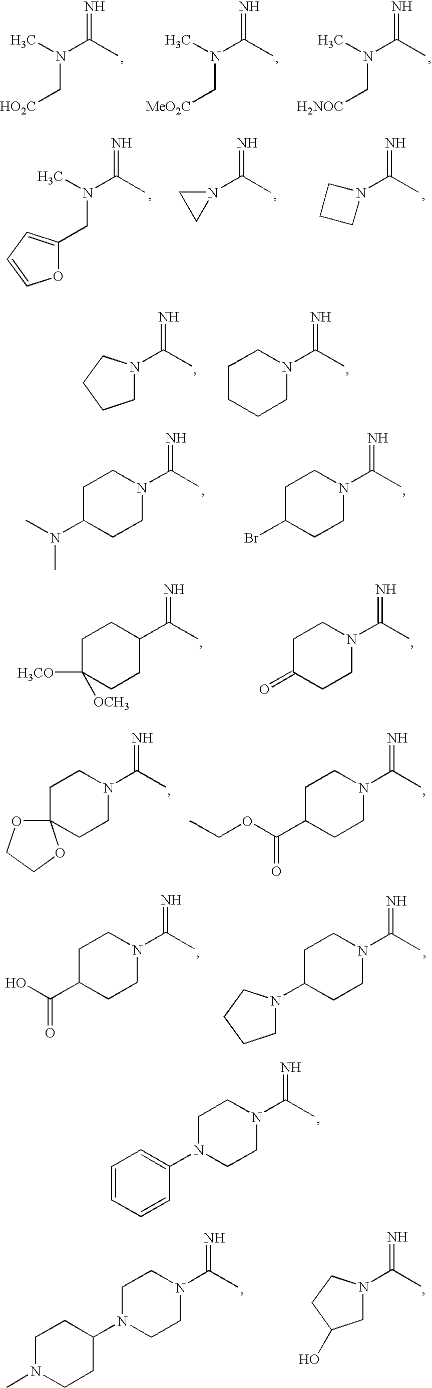

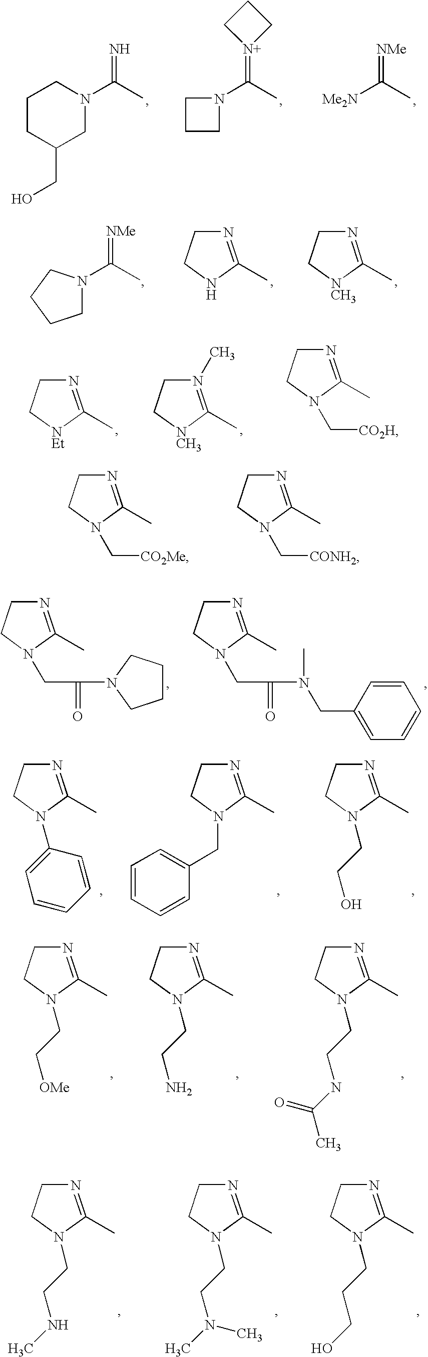

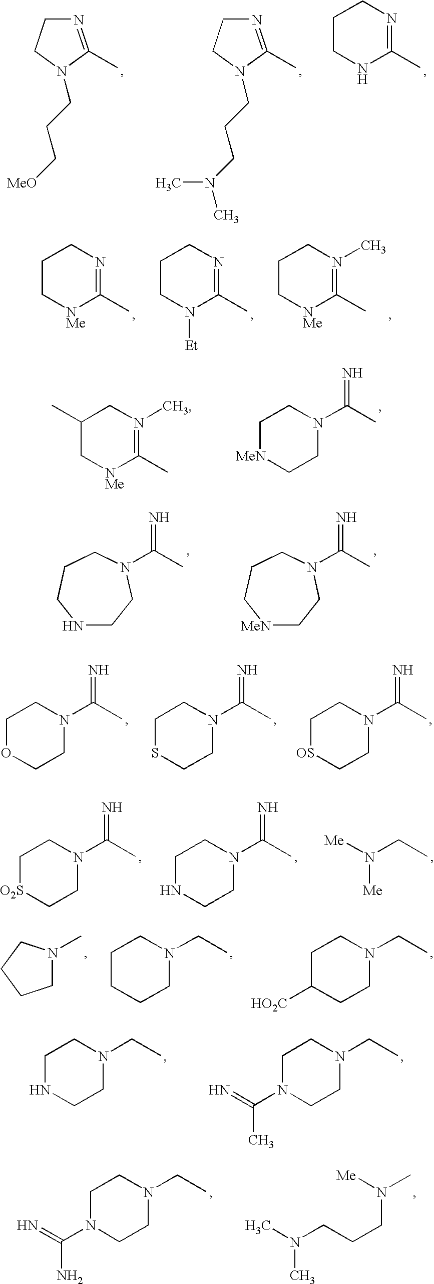

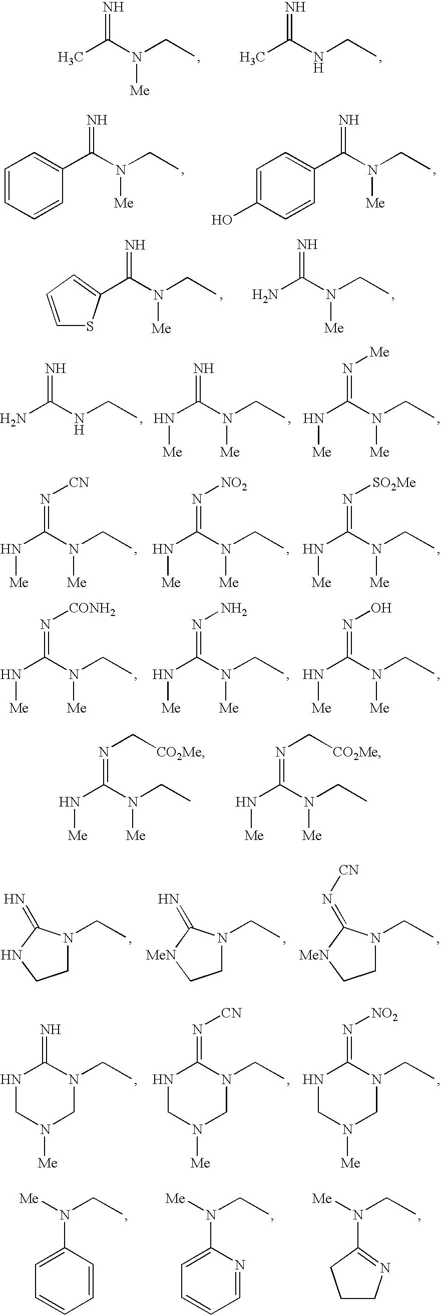

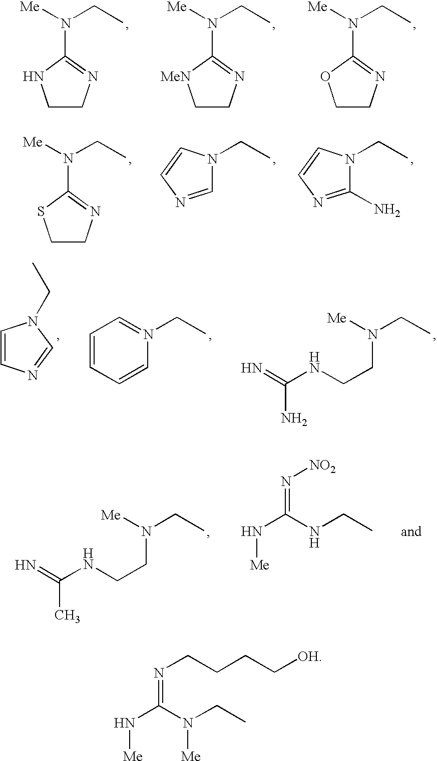

11. A compound of claim 1 of formula (I) having the following structure: 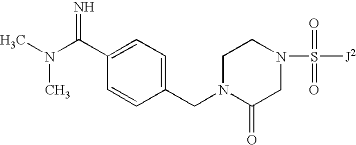
wherein:
J² is a member selected from the group consisting of:

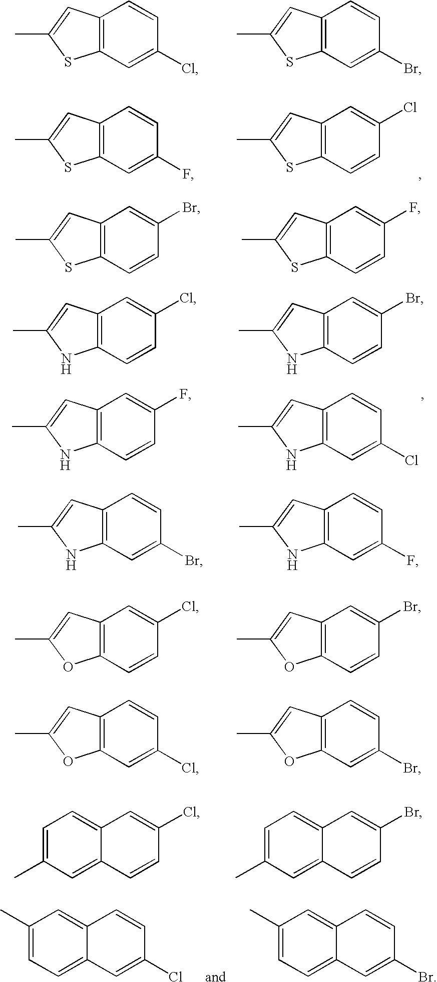

12. A compound of claim 1 of formula (II) having the following structure: 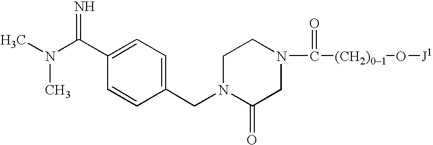
wherein:
J¹ is a member selected from the group consisting of:

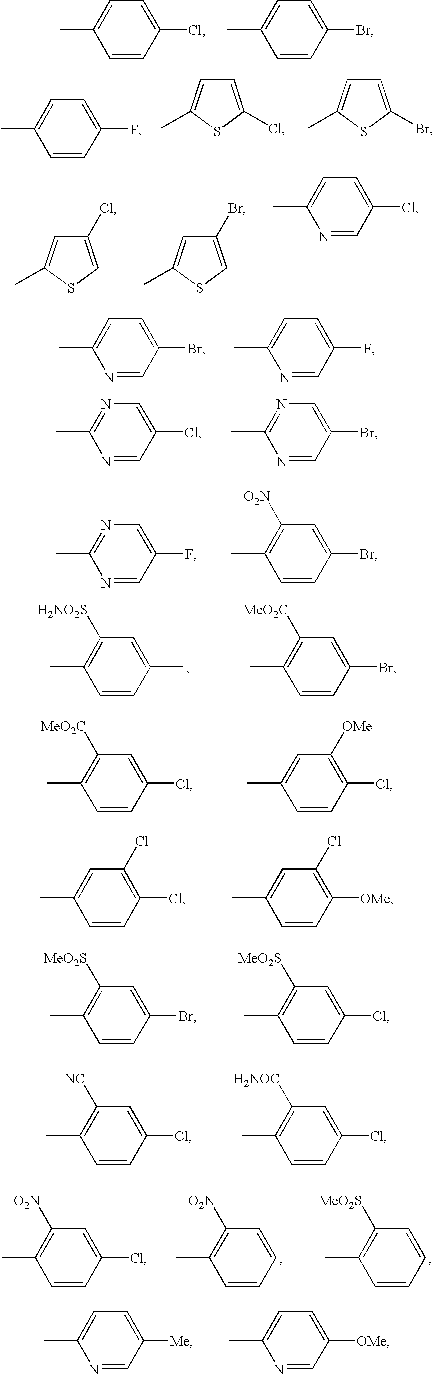

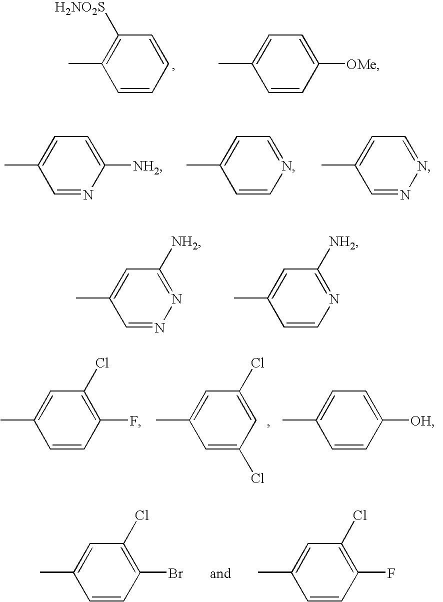

13. A compound selected from the group consisting of: 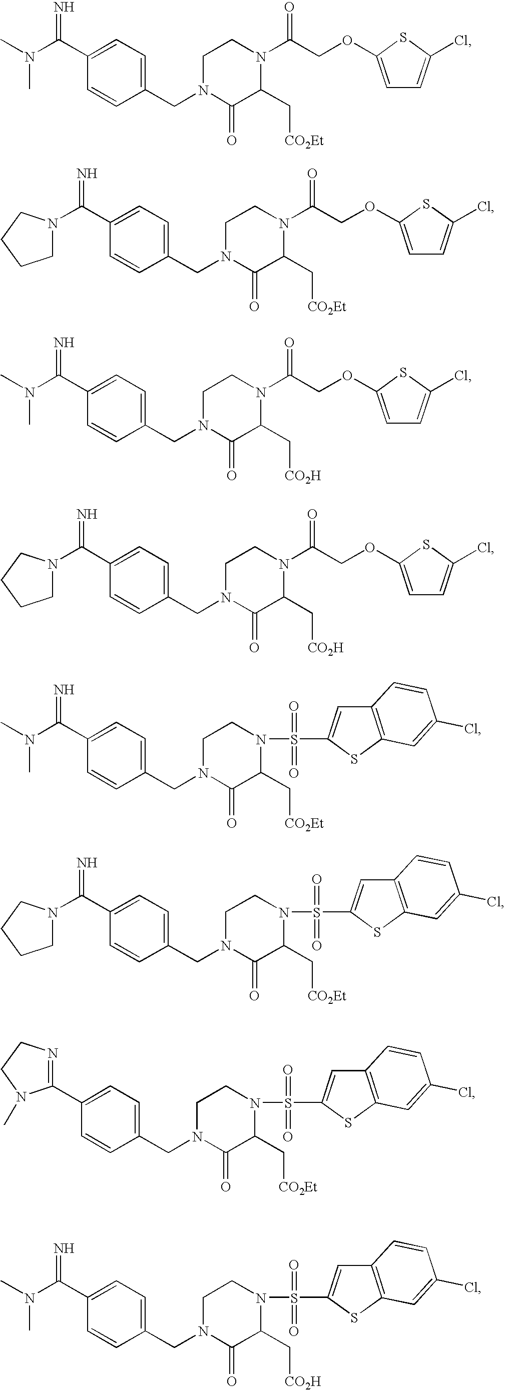 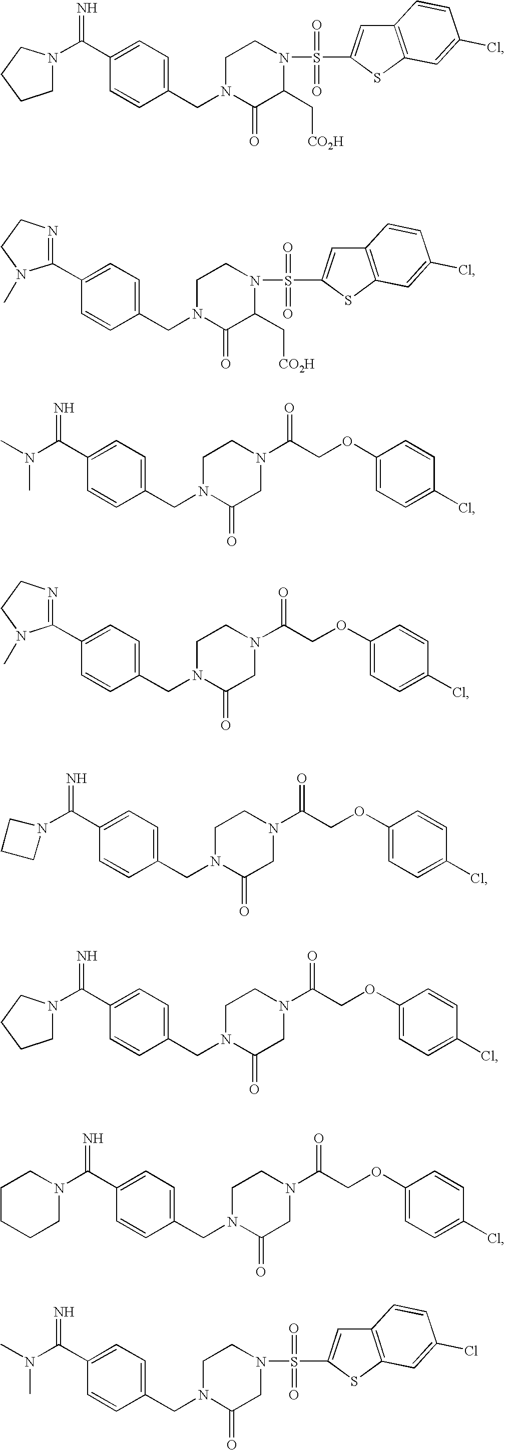 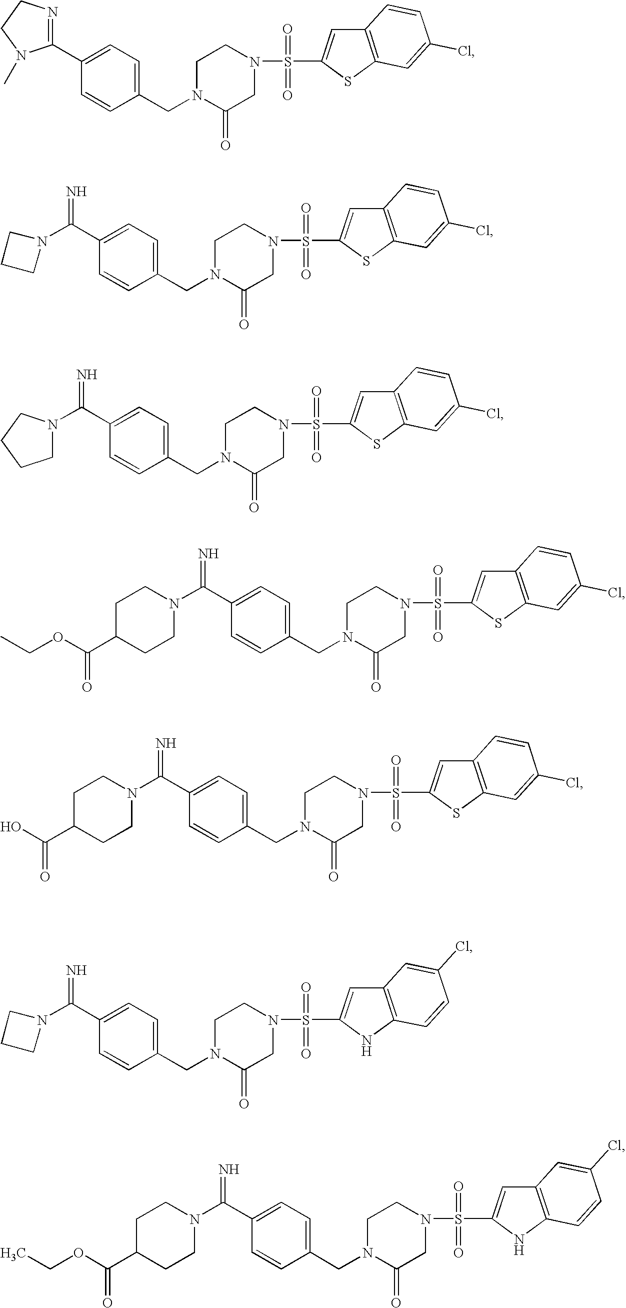 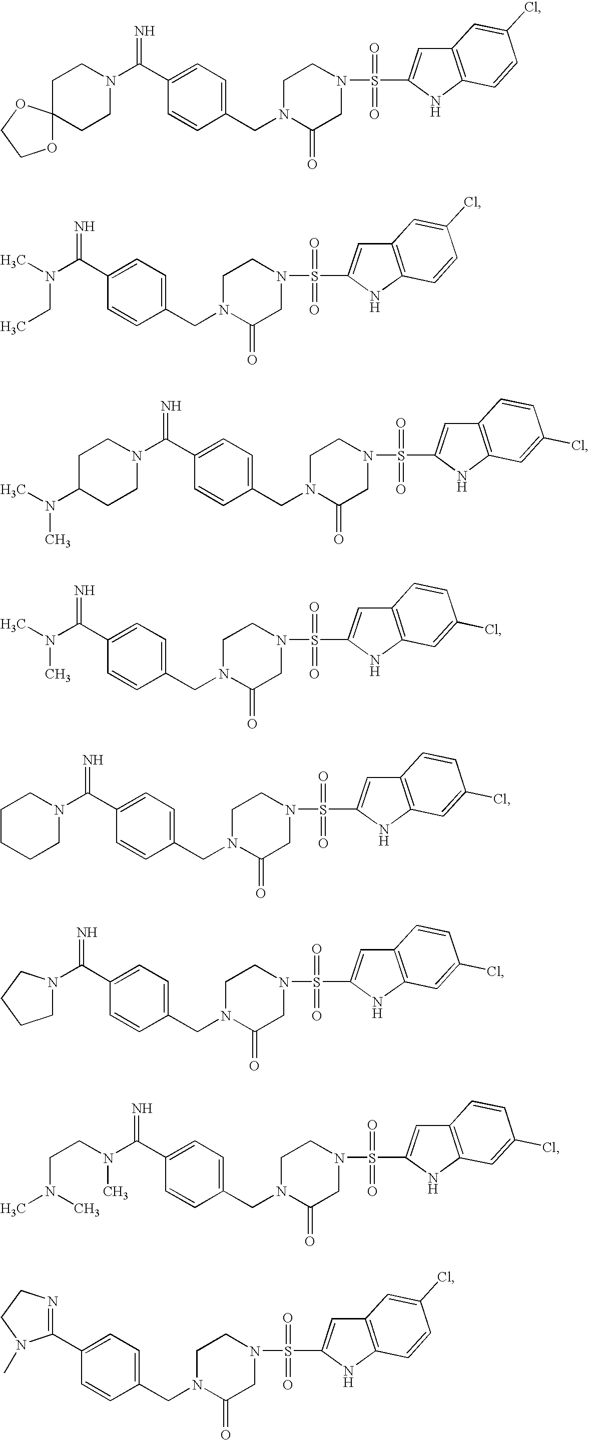 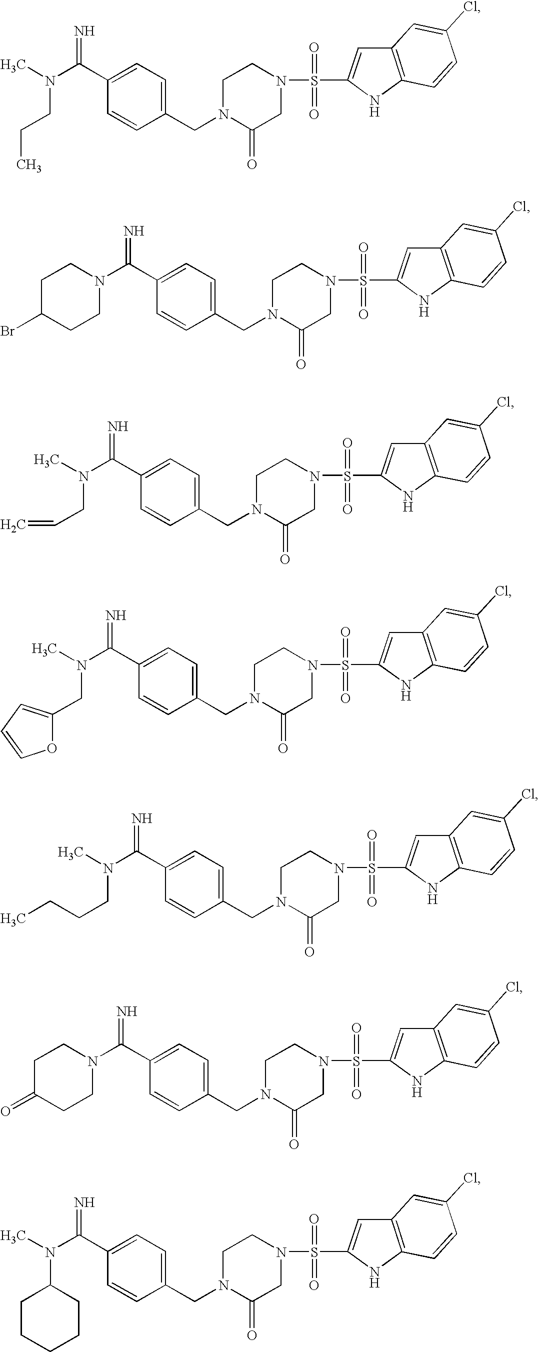 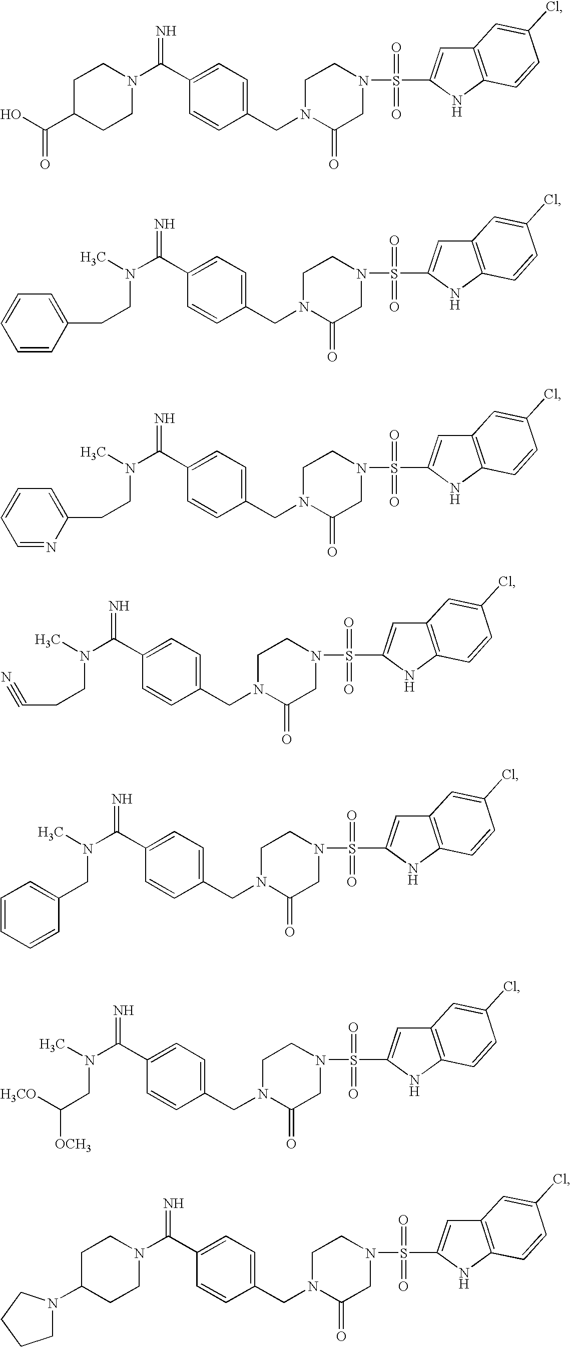 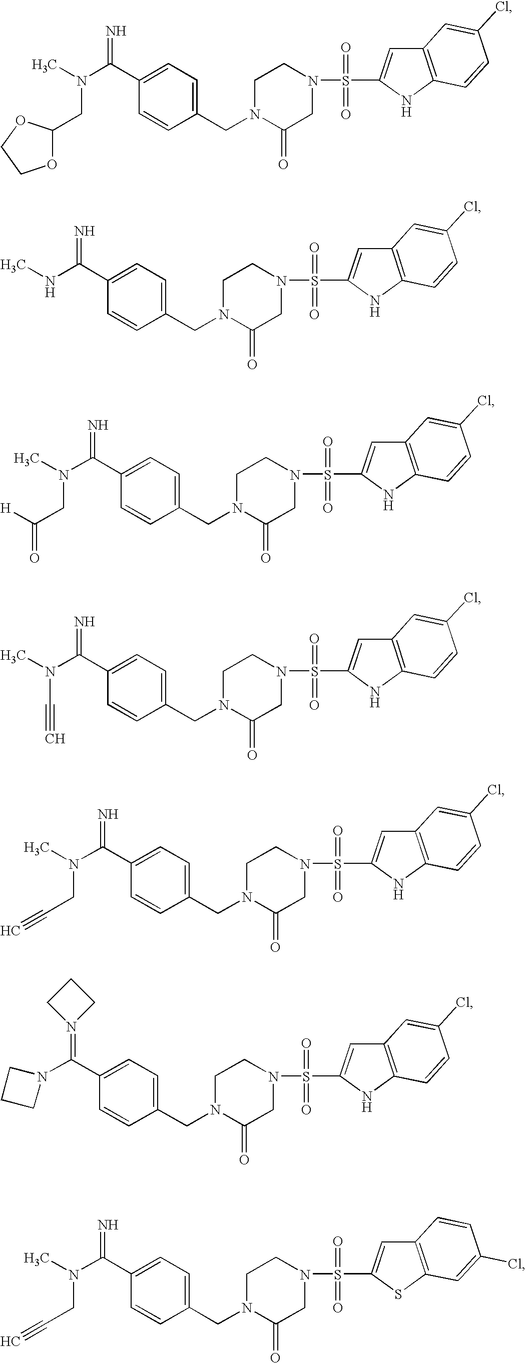 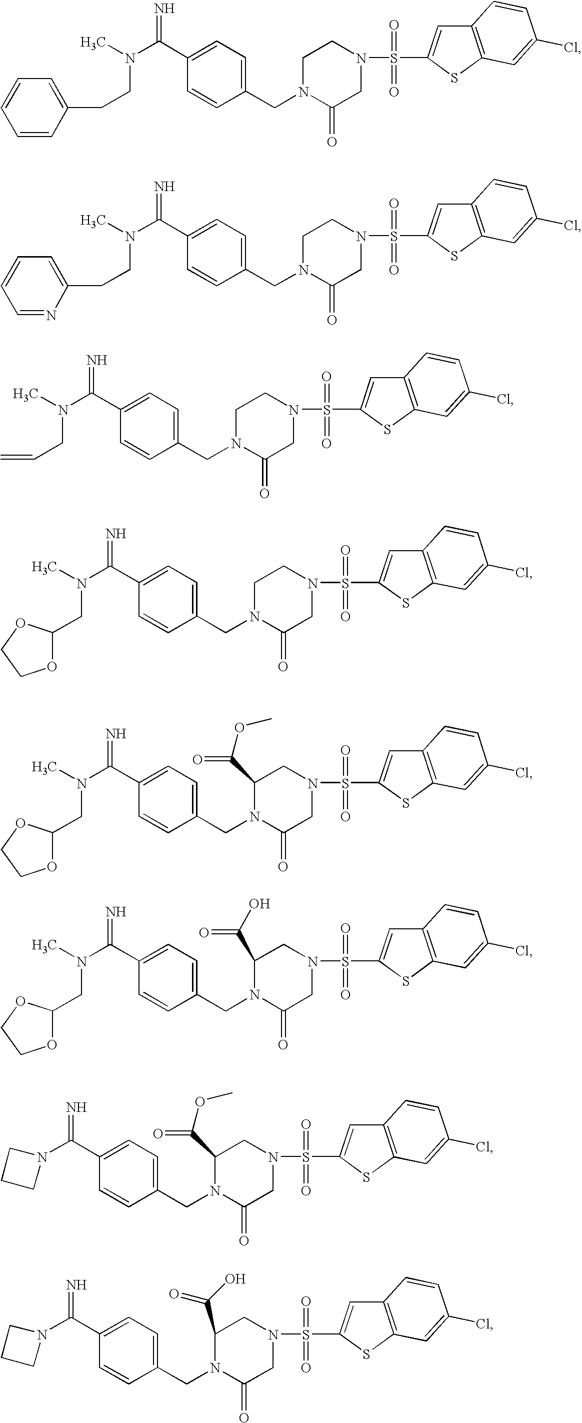 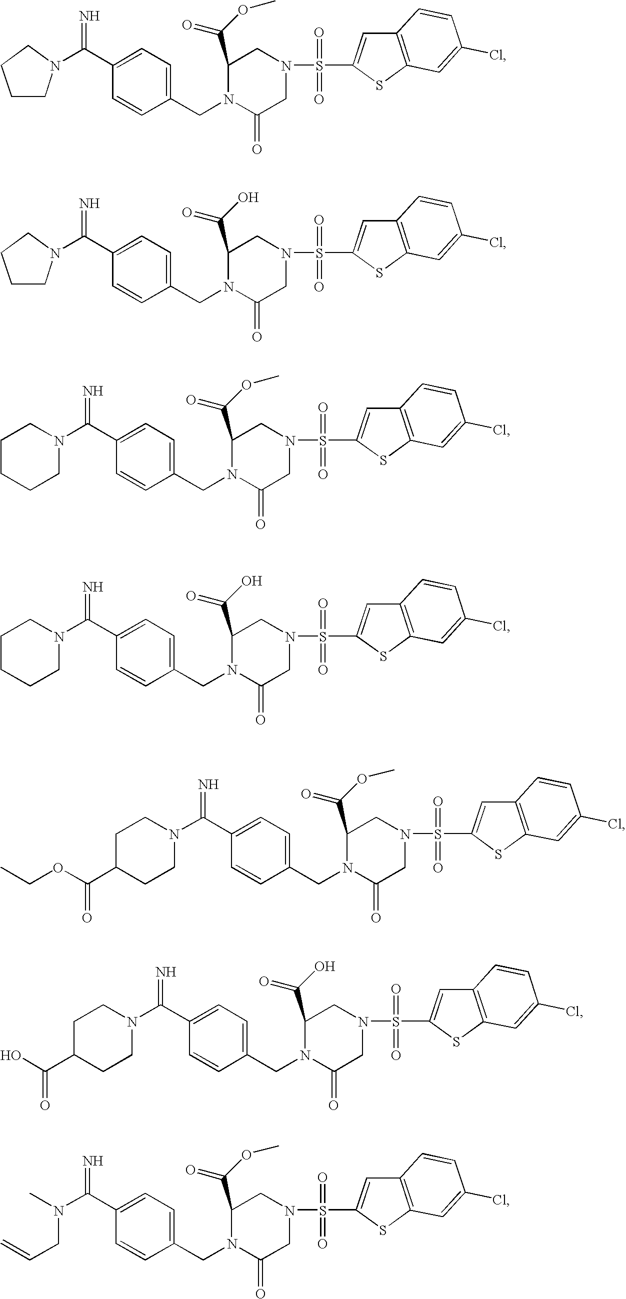 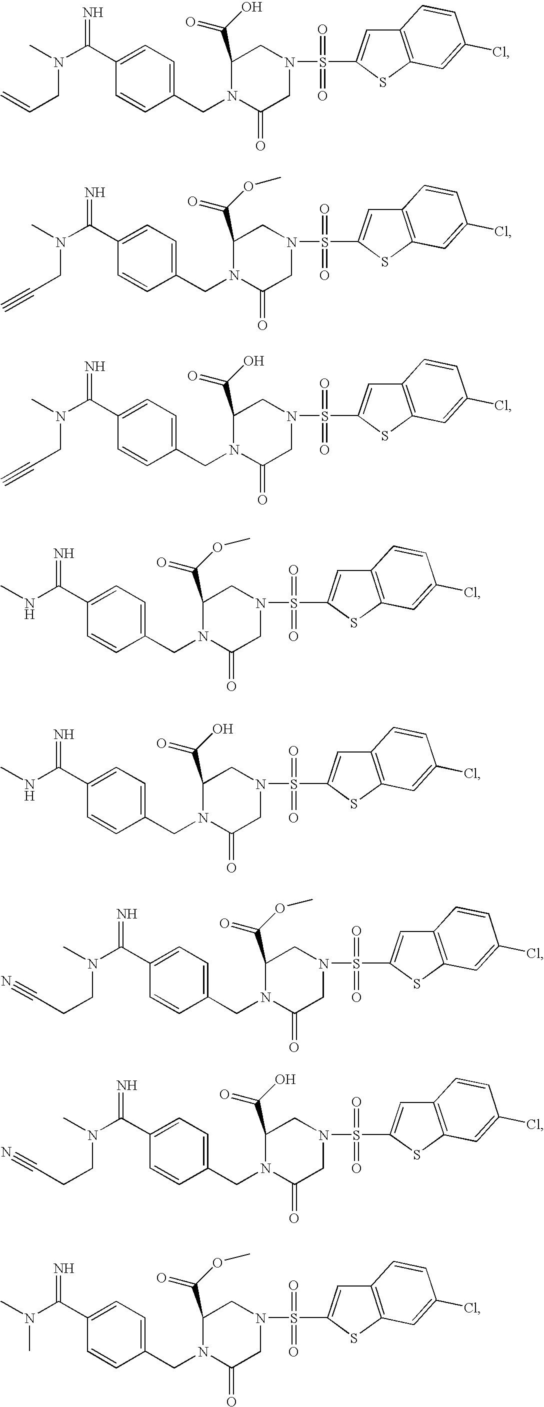 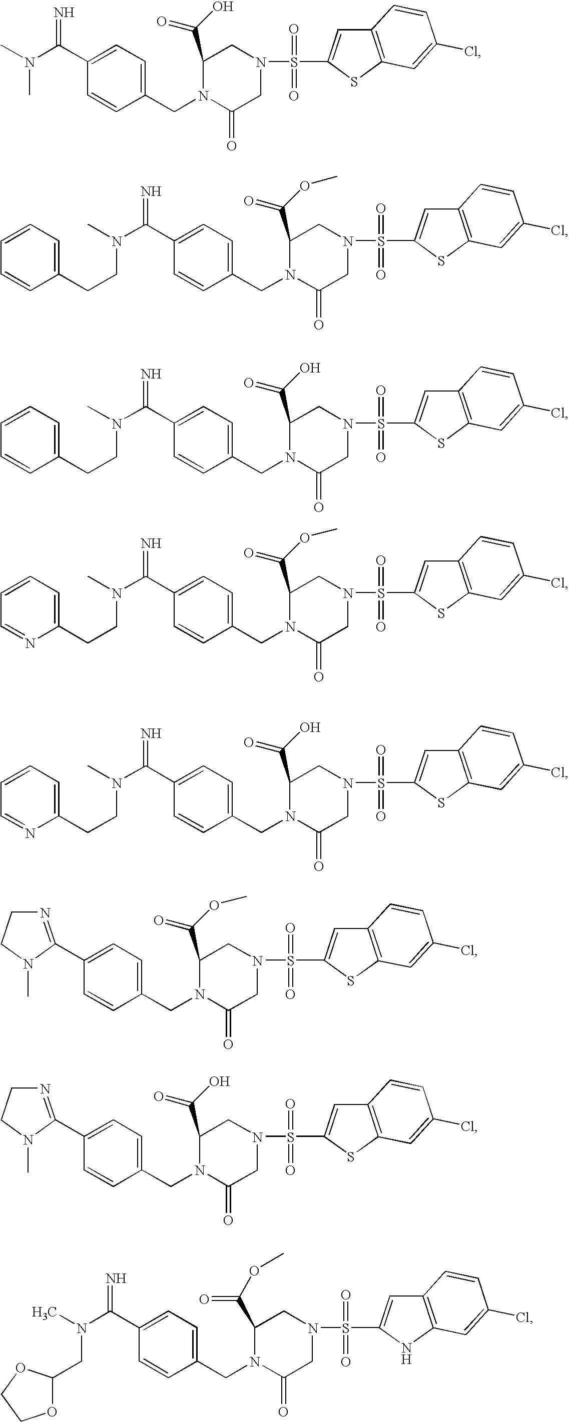 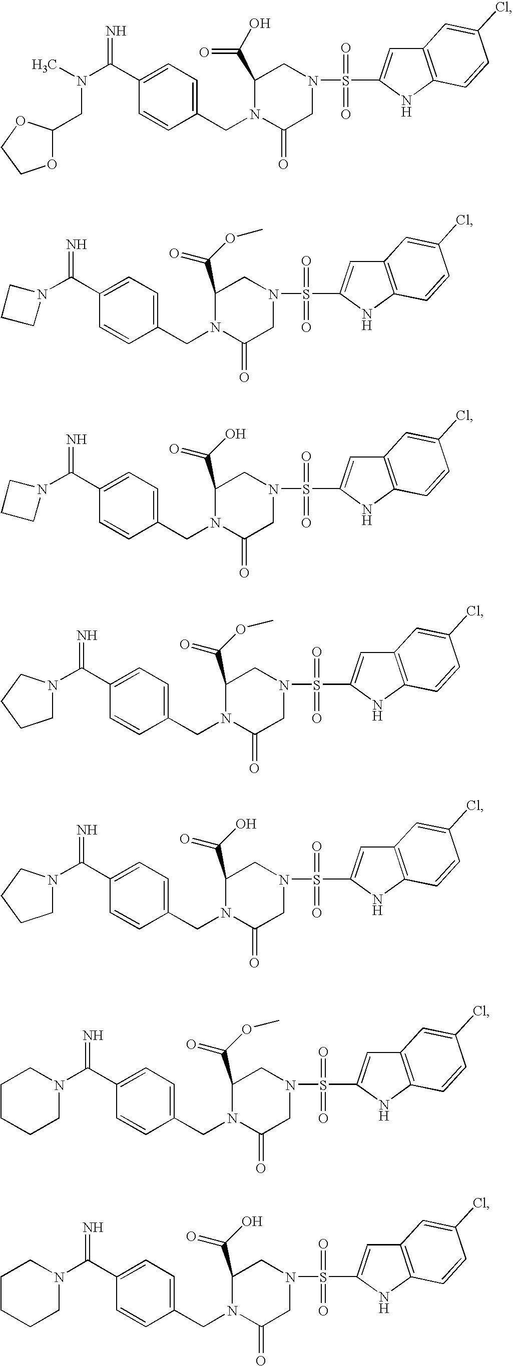 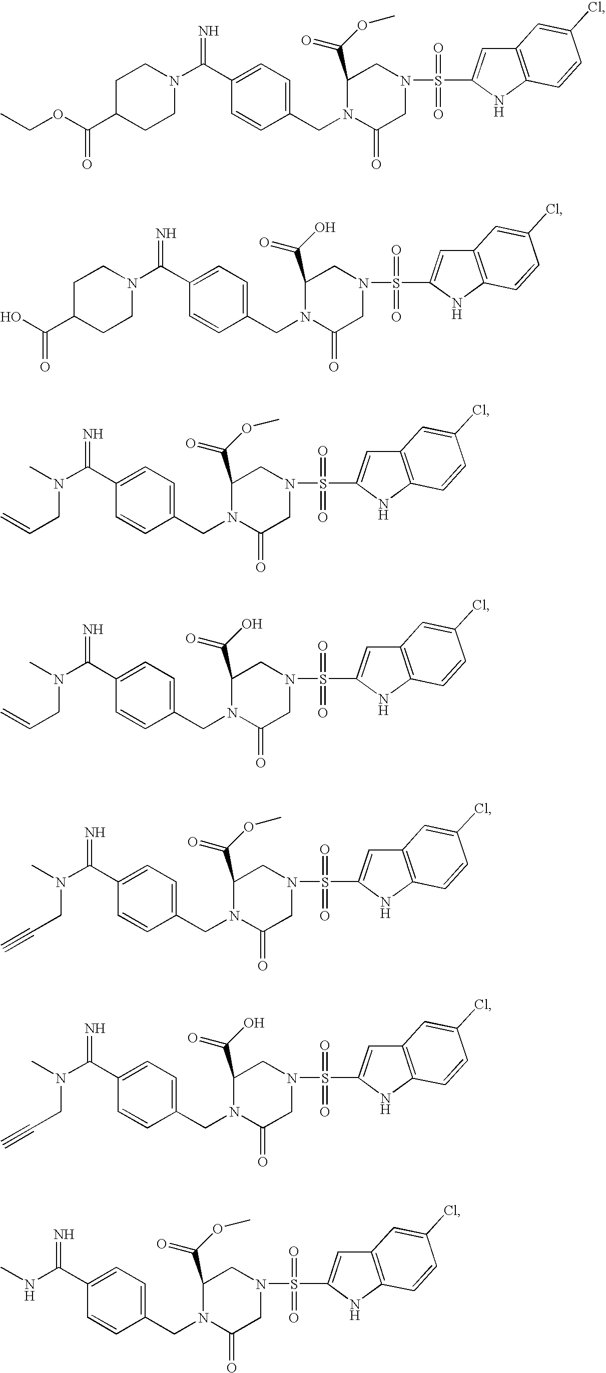 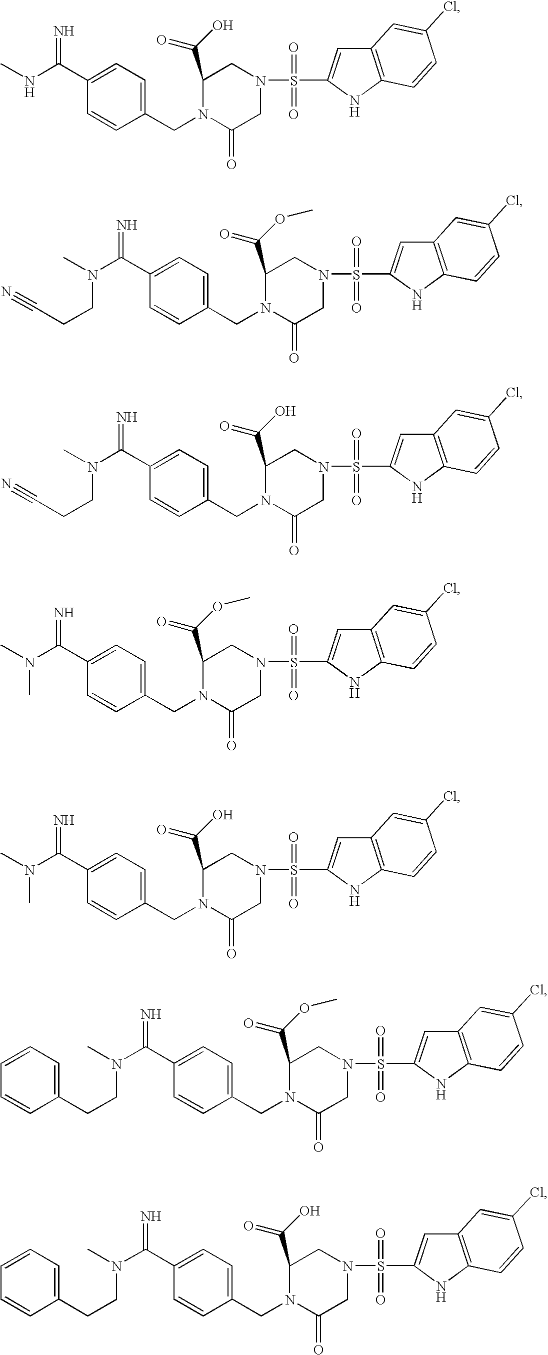 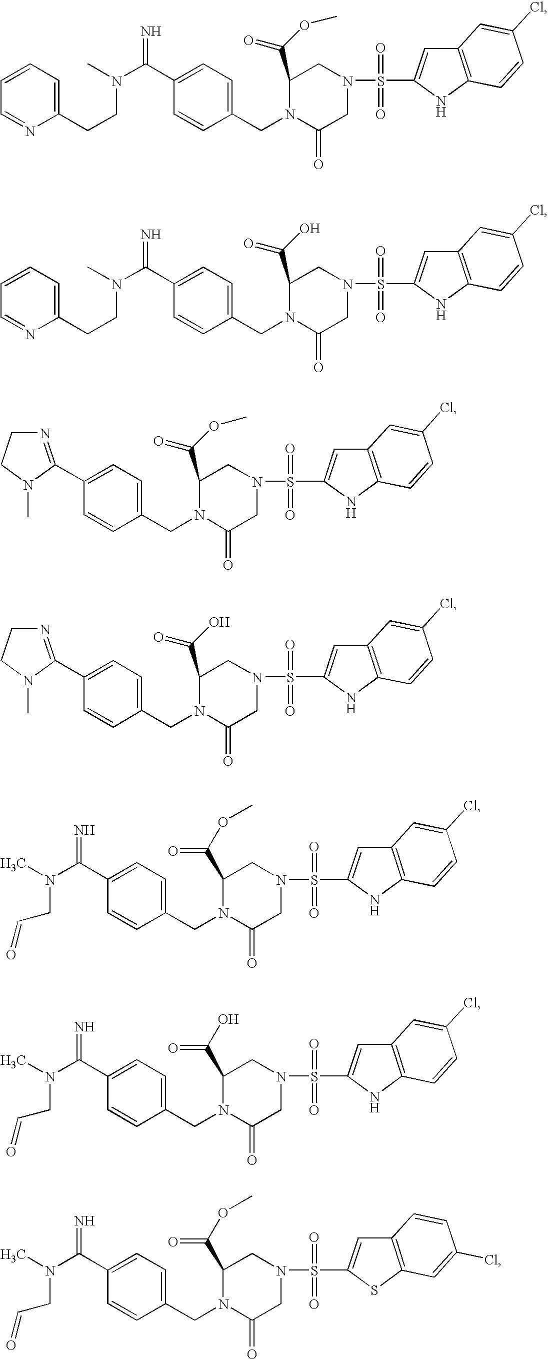 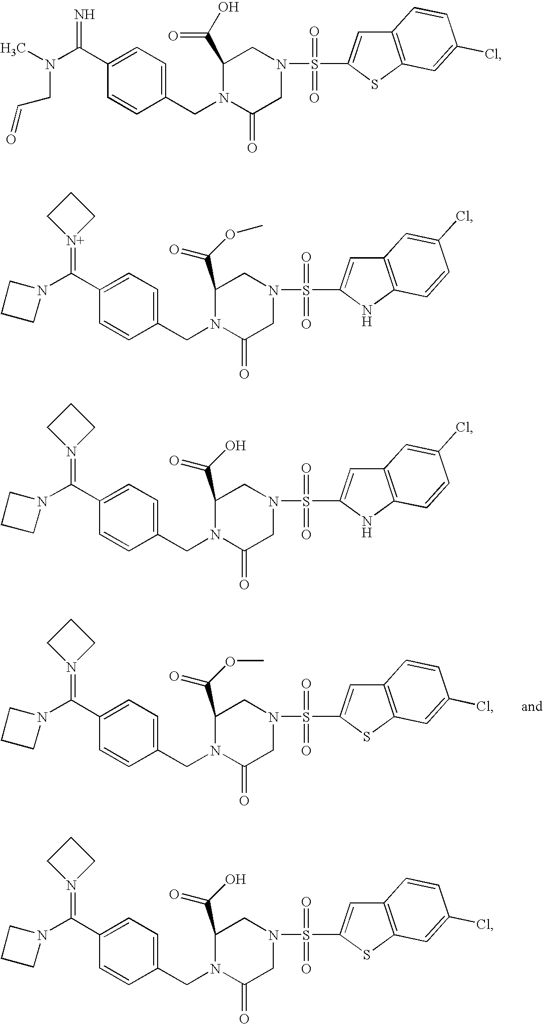
and all pharmaceutically acceptable isomers, salts, hydrates, solvates and prodrug derivatives thereof.

14. A pharmaceutical composition for preventing or treating a condition in a mammal characterized by undesired thrombosis comprising a pharmaceutically acceptable carrier and a therapeutically effective amount of a compound of one of claims 1-13.

15. A method for preventing or treating a condition in a mammal characterized by undesired thrombosis comprising administering to said mammal a therapeutically effective amount of a compound of one of claims 1-13.

16. The method of claim 15, wherein the condition is selected from the group consisting of:
acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

17. A method for inhibiting the coagulation of biological samples comprising the administration of a compound of one of claims 1-13.
